(19) United States
(12) Patent Application Publication
(10) Pub. No.: US 2024/0032861 A1
(43) Pub. Date: Feb. 1, 2024

(54) SLEEP SYSTEM WITH PERSONALIZED SLEEP RECOMMENDATIONS BASED ON CIRCADIAN CHRONOTYPE

(71) Applicant: Sleep Number Corporation, Minneapolis, MN (US)

(72) Inventors: Stacy Marie Stusynski, Blaine, MN (US); Kristen Elizabeth Swenby, Blaine, MN (US); Carol Lynn Herman, Minneapolis, MN (US)

(21) Appl. No.: 18/223,917

(22) Filed: Jul. 19, 2023

Related U.S. Application Data
(60) Provisional application No. 63/393,292, filed on Jul. 29, 2022.

(51) Int. Cl. A61B 5/00; G16H 10/60; G16H 10/20; G16H 50/20; G16H 20/30; G16H 20/60

(52) U.S. Cl. CPC A61B 5/4815; G16H 10/60; G16H 10/20; G16H 50/20; G16H 20/30; G16H 20/60; A61B 5/7475

(57) ABSTRACT

Disclosed are systems, methods, and techniques for improving a sleep quality and/or health metric of a user of a bed system. A computer system can be in communication with a user device of the user. The computer system can transmit, to the user device, a subjective sleep health questionnaire for presentation in a graphical user interface (GUI) display, receive, from the user device, user input indicating a response to the subjective sleep health questionnaire, process the user input to determine a circadian chronotype of the user, identify, based on the circadian chronotype of the user, at least one insight for presentation in the GUI display at the user device, and transmit, to the user device, the at least one insight for presentation in the GUI display. The computer system can also determine the circadian chronotype of the user based on sensor data collected by sensors of the user's bed system.

### CROSS-REFERENCE TO RELATED APPLICATIONS

This application claims the benefit of U.S. Provisional Application Ser. No. 63/393,292, filed Jul. 29, 2022. The disclosure of the prior application is considered part of the disclosure of this application, and is incorporated in its entirety into this application.

### TECHNICAL FIELD

The present document relates to automated sensing of sleep quality and recommendations for improvement.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

The document generally describes technology for providing personalized insights and recommendations to a user to improve their sleep quality and/or health. More particularly, the disclosed technology provides for presenting a sleep health questionnaire in a graphical user interface (GUI) display at a user device. A computer system can receive user input to the questionnaire and, based on the user input, determine a circadian chronotype of the user. The computer system can then identify and present personalized insights, such as sleep tips, habit recommendations, routine changes, and/or other behaviors, to the user that correspond to the user's circadian chronotype. The circadian chronotype can correspond to habits of a night owl, habits of an early bird, and/or neutral habits. Moreover, the computer system can leverage existing data about the user and/or a population of users, which can be derived from sensors of a bed system and/or wearable devices collecting various data metrics about the user(s), to determine a circadian chronotype of the user and/or deliver insights that enable subjective and emotional responses from the user. For example, the user can receive enhanced personalized and general sleep health insights based on their selected or other inputted responses to the questionnaire as well as system-automated analysis of historic sleep patterns and/or behavior of the user. Tips can be presented with the insights that specifically address topics from the questionnaire or other aspects of the user's circadian rhythm. Such tips can be surfaced at one or more GUI displays at the user device using randomized logic and/or intelligent machine learning rules/logic that is configured to determine relevance of the tips to the user's experience, sleep patterns, and/or sleep quality and/or health.

The insights selected for and presented to the user in a graphical user interface (GUI) display at a user device can include recommendations to deepen and/or start particular habits that improve the user's sleep quality and/or health. The insights can correspond to the user's circadian chronotype, stress levels (e.g., subjective user-perceived and/or objective sensor-measured), and/or ability to fall asleep and stay asleep (e.g., subjective user-perceived and/or objective sensor-measured). The insights may also deliver educational benefits about various multi-dimensional factors of sleep quality. The computer system can select insights that include tips and/or recommendations to link activities of the user on a current day (or other period of time) with getting better sleep on a subsequent day or days (or other future period of time).

A system of one or more computers can be configured to perform particular operations or actions described herein by virtue of having software, firmware, hardware, or a combination of them installed on the system that in operation causes or cause the system to perform the actions. One or more computer programs can be configured to perform particular operations or actions described herein by virtue of including instructions that, when executed by data processing apparatus, cause the apparatus to perform the actions.

One or more embodiments described herein can include a system for improving at least one of a sleep quality and a health metric of a user, the system can include a computer system in communication with a user device of a user. The computer system can be configured to: transmit, to the user device, a subjective sleep health questionnaire for presentation in a graphical user interface (GUI) display, receive, from the user device, user input indicating at least one response to the subjective sleep health questionnaire, process the user input to determine a circadian chronotype of the user, identify, based on the circadian chronotype of the user, at least one insight for presentation in the GUI display at the user device, and transmit, to the user device, the at least one insight for presentation in the GUI display.

In some implementations, the embodiments described herein can optionally include one or more of the following features. For example, processing the user input to determine a circadian chronotype of the user can include: retrieving, from a data store, a lookup table that maps responses to the subjective sleep health questionnaire to circadian chronotypes, and selecting the circadian chronotype that matches the at least one response from the user. The computer system can also transmit the subjective sleep health questionnaire to the user device based on receiving an indication, from the user device, that the user registered an account with the computer system. In some implementations, the at least one insight can be a behavior recommendation to maintain a current circadian rhythm of the user. The at least one insight can be a behavior recommendation to improve a current circadian rhythm of the user over a predetermined period of time. The predetermined period of time can be a next sleep session. The predetermined period of time can be a threshold quantity of successive sleep sessions. The threshold quantity can be 5 successive sleep sessions. The threshold quantity can be 7 successive sleep sessions. The at least one insight can be a behavior recommendation to reduce a current level of stress of the user by a threshold amount. The at least one insight can be a behavior recommendation to improve an ability of the user to fall asleep faster by a threshold amount. The at least one insight can be a behavior recommendation to improve an ability of the user to stay asleep during a sleep session.

As another example, identifying the at least one insight can include: retrieving, from a data store, sleep and health insights associated with the circadian chronotype of the user, identifying, based on the processed user input, a subset of insights from the retrieved sleep and health insights, and selecting, using a randomization technique, the at least one insight from the subset of insights for presentation to the user. The randomization technique can be a random number generator. In some implementations, the questionnaire can prompt the user for user input about at least one of user-perceived circadian rhythm, user-perceived stress, user-perceived trouble falling asleep, and user-perceived trouble staying asleep. The at least one insight can include a recommendation of when the user should exercise. The at least one insight can include a recommendation of when the user may be most alert during a predetermined period of time. The predetermined period of time can be an amount of time between two successive sleep sessions. The predetermined period of time can be an amount of time between when the user wakes up from a first sleep session and when the user goes to sleep in a second sleep session. The at least one insight can include a recommendation of when the user should eat a meal. The at least one insight can include a recommendation of when the user should begin a bedtime routine. The at least one insight can include a notification prompting the user to perform an activity that may improve at least one of a current health metric and a current sleep quality of the user.

As another example, the subjective sleep health questionnaire can prompt the user for an indication of whether the user may be an early bird, a night owl, or neutral. The subjective sleep health questionnaire can prompt the user for an indication of at least one factor that may disrupt sleep patterns of the user. The subjective sleep health questionnaire can prompt the user for an indication of frequency of at least one sleep problem. The at least one sleep problem can include at least one of the group consisting of trouble falling asleep, waking up during a sleep session, returning to sleep after waking up during the sleep session, and waking up too early and unable to fall asleep. In some implementations, the subjective sleep health questionnaire can prompt the user for an indication of at least one sleep condition for which the user may be diagnosed. The subjective sleep health questionnaire can also prompt the user for an indication of how frequently the user may use at least one sleep-aid device.

In some implementations, the transmitting, to the user device, the subjective sleep health questionnaire for presentation in the GUI display can be performed a single instance. The computer system can also be configured to receive, from the user device, updated user input to the subjective sleep health questionnaire at a later time than when the computer system received the user input. The at least one insight can be a personalized insight that may correspond to historic sleep and health data associated with the user. The at least one insight can also be a general insight that may correspond to sleep and health data of a population of users that includes the user. The subjective sleep health questionnaire can be presented in a first screen in the GUI display and the at least one insight can be presented in a second screen in the GUI display, the second screen can be different than the first screen.

As another example, the at least one insight can include a tip about the circadian chronotype of the user and at least one behavior recommendation to improve the sleep quality or health metric of the user. The tip and the at least one behavior recommendation can be presented concurrently in the GUI display, the GUI display being scrollable in a vertical direction. In response to receiving user input indicating a scrolling feature from the user device, the computer system can be configured to transmit instructions, to the user device, that can cause the user device to present at least a portion of the tip and the at least one behavior recommendation concurrently in the GUI display. Another portion of at least one of the tip and the at least one behavior recommendation may no longer be visible in the GUI display. The at least one insight further can include a selectable option to generate daily routines based on the at least one behavior recommendation, the selectable option being presented concurrently in the GUI display with the tip and the at least one behavior recommendation. In response to receiving user input indicating selection of the selectable option from the user device, the computer system can also be configured to transmit instructions, to the user device, that can cause the user device to present another screen in the GUI display that may replace the presentation of the tip, the at least one behavior recommendation, and the selectable option.

The at least one behavior recommendation can also be presented in the GUI display with a graphical element corresponding to a type of the at least one behavior recommendation. For example, the type of the at least one behavior recommendation can be a time-to-exercise recommendation and the corresponding graphical element can be exercise equipment, the type of the at least one behavior recommendation can be a most-alert recommendation and the corresponding graphical element can be a lightbulb, the type of the at least one behavior recommendation can be a time-to-eat recommendation and the corresponding graphical element can be a utensil, and the type of the at least one behavior recommendation can be a time-to-prepare-for-bed recommendation and the corresponding graphical element can be a coffee cup. In some implementations, at least one behavior recommendation can be presented in the GUI display with an indication of threshold period of time for performing the at least one behavior recommendation, the threshold period of time indicating a range of time at which performing the at least one behavior recommendation can improve at least one of the sleep quality and the health metric of the user.

As another example, the system can also include a bed having at least one sensor that can be configured to sense physical phenomenon of the user on the bed. The bed can include a controller, and the computer system can be the controller. The computer system can be part of the bed. The computer system can be remote from the bed. The bed can include a mattress with at least one air chamber, and the at least one sensor can be a pressure sensor in fluid communication with the air chamber. The system can also include means for controlling pressure of the bed that can include the at least one sensor. The computer system can be a server physically separate from the bed and the user device and connected to the bed and the user device by a data network. The at least one sensor can be at least one of a pressure sensor, a temperature sensor, a load cell, and a humidity sensor.

In some implementations, the computer system can be configured to receive, from the at least one sensor, sensor readings during a sleep session of the user, process the sensor readings to determine at least one of an objective sleep quality and an objective health metric, and identify the subset of insights based at least in part on the processed user input and the processed sensor readings. The computer system can also be configured to receive, from the at least one sensor, sensor readings during a sleep session of the user, process the sensor readings to determine at least one of an objective sleep quality and an objective health metric, and select the at least one insight from the subset of insights based at least in part on the processed sensor readings. In some implementations, the computer system can receive, from the at least one sensor, sensor readings during a sleep session of the user, and process the sensor readings to determine a proposed circadian chronotype of the user. The computer system can also validate the proposed circadian chronotype of the user with the circadian chronotype determined based on the user input.

In some implementations, to select at least one insight from the subset of insights, the computer system can assemble the subset of insights from a template of general insights that have been completed with information generic to a population of users that may include the user. To select at least one insight from the subset of insights, the computer system can assemble the subset of insights from a template of personalized insights that have been completed with information specific to the user. Sometimes, the computer system can be a cloud computing system. The computer system can also be the user device of the user. The computer system can be a home-automation hub.

In some implementations, the computer system can also retrieve, from a data store, historic sleep data about the user, and apply a ruleset to the historic sleep data to determine a proposed circadian chronotype of the user. The computer system can also validate the proposed circadian chronotype of the user with the circadian chronotype determined based on the user input. The retrieving and applying steps can be performed before the transmitting, to the user device, the sleep health questionnaire step. The retrieving and applying steps can also be performed after at least one of the transmitting, to the user device, the sleep health questionnaire step and the processing step. The historic sleep data can include sleep patterns and behavior of the user over a threshold period of time. The circadian chronotype can be at least one of a night owl, an early bird, and neutral. The at least one insight can be a reminder to go to bed within a predetermined time. The circadian chronotype can be at least one of a first type, a second type, and a third type. The first type can be a night owl, the second type can be an early bird, and the third type can be neutral. The circadian chronotype can also be at least one of a first type and a second type. The first type can be a night owl and the second type can be an early bird.

One or more embodiments described herein can include a system for improving at least one of a sleep quality and a health metric of a user, the system having: a computer system in communication with a user device of a user, the computer system being configured to: retrieve, from a data store, sleep patterns and behavior data for the user, apply a ruleset to the retrieved data to determine a circadian chronotype of the user, determine at least one insight, tip, or reminder for the user, the at least one insight, tip, or reminder corresponding to the circadian chronotype of the user, and return at least one of (i) the circadian chronotype and (ii) the determined insight, tip, or reminder.

The system can optionally include one or more of the following features. For example, the returning step can include transmitting, to the user device, at least one of (i) and (ii) for presentation in a GUI display of the user device. The returning step can include generating instructions to control a component of a bed system or a peripheral device according to the at least one insight, tip, and reminder. Determining the circadian chronotype of the user can include mapping the sleep patterns and behavior data for the user to at least one circadian chronotype defined by the ruleset. The computer system can also be further configured to: output, in a GUI display of the user device, the circadian chronotype of the user, receive user input, from the user device, indicating verification of the circadian chronotype, and in response to receiving the user input, perform the determining step. The computer system may also: output, in a GUI display of the user device, the circadian chronotype of the user, receive user input, from the user device, indicating a user-perceived circadian chronotype that may be different than the outputted circadian chronotype, and in response to receiving the user input, determine the at least one insight, tip, and reminder for the user that may correspond to the user-perceived circadian chronotype.

One or more embodiments described herein can include a system for improving at least one of a sleep quality and a health metric of a user, the system having: a computer system in communication with a user device of a user, the computer system being configured to: present a sleep health questionnaire at the user device, receive user input, from the user device, indicating at least one response to the questionnaire, determine, based on the user input, a proposed circadian chronotype of the user, retrieve, from a data store, sleep patterns and behavior data for the user, determine whether the retrieved sleep patterns and behavior data corresponds to the proposed circadian chronotype of the user, determine, based on the retrieved sleep patterns and behavior data corresponding to the proposed circadian chronotype, at least one insight, tip, or reminder for the user, the determined insight, tip, or reminder corresponding to the proposed circadian chronotype, and return at least one of (i) the proposed circadian chronotype and (ii) the insight, tip, or reminder.

The system can optionally include one or more of the following features. For example, the computer system can also determine, based on the retrieved data not corresponding to the proposed circadian chronotype, another circadian chronotype of the user based on the retrieved data, determine at least one insight, tip, and reminder for the user that may correspond to the another circadian chronotype, and return at least one of (i) the another circadian chronotype and (ii) the insight, tip, or reminder that corresponds to the another circadian chronotype.

One or more embodiments described herein can include a system that can be configured to generate an output based on a user circadian chronotype. The system can optionally include one or more of the abovementioned features.

One or more embodiments described herein can include a system that can be configured to determine a user circadian chronotype based on user input to a questionnaire. The system can optionally include one or more of the abovementioned features.

One or more embodiments described herein can include a system that can be configured to determine a user circadian chronotype based on historic sleep patterns and behavior data of the user. The system can optionally include one or more of the abovementioned features.

One or more embodiments described herein can include a system that can determine a user circadian chronotype based on a combination of historic sleep patterns and behavior data of the user and user input to a questionnaire. The system can optionally include one or more of the abovementioned features.

One or more embodiments described herein can include a system to determine a user circadian chronotype and generate output based on the determined circadian chronotype of the user. The system can optionally include one or more of the abovementioned features.

One or more embodiments described herein can include a system to: receive, from sensors of a bed system, sensor data during a sleep session of a user, receive, from a user device of the user, user input indicating responses to a questionnaire, determine, based at least in part on the sensor data and the user input, a circadian chronotype of the user, generate at least one insight, tip, or recommendation based on the circadian chronotype of the user, and transmit the at least one insight, tip, or recommendation to the user device for presentation in a GUI display of the user device. The system can optionally include one or more of the abovementioned features.

One or more embodiments described herein can include a system that can: retrieve, from a data store, historic sleep patterns and behavior data a user, receive, from a user device of the user, user input indicating responses to a questionnaire, determine, based at least in part on the retrieved historic sleep patterns and behavior data and the user input, a circadian chronotype of the user, generate at least one insight, tip, or recommendation based on the circadian chronotype of the user, and transmit the at least one insight, tip, or recommendation to the user device for presentation in a GUI display of the user device. The system can optionally include one or more of the abovementioned features.

One or more embodiments described herein can include a method for identifying behavior recommendations for improving at least one of a sleep quality and a health metric of a user, the method including: transmitting, by a computing system to a user device, a subjective sleep health questionnaire for presentation in a graphical user interface (GUI) display of the user device, receiving, by the computing system from the user device, user input indicating at least one response to the subjective sleep health questionnaire, processing, by the computing system, the user input to determine a circadian chronotype of the user, identifying, by the computing system and based on the circadian chronotype of the user, at least one insight for presentation in the GUI display at the user device, and transmitting, by the computing device to the user device, the at least one insight for presentation in the GUI display of the user device. The method can optionally include one or more of the abovementioned features.

One or more embodiments described herein can include a method for identifying behavior recommendations for improving at least one of a sleep quality and a health metric of a user, the method including: retrieving, by a computing system from a data store, sleep patterns and behavior data for the user, applying, by the computing system, a ruleset to the retrieved data to determine a circadian chronotype of the user, determining, by the computing system, at least one insight, tip, or reminder for the user, the at least one insight, tip, or reminder corresponding to the circadian chronotype of the user, and returning, by the computing system, at least one of (i) the circadian chronotype and (ii) the determined insight, tip, or reminder. The method can optionally include one or more of the abovementioned features.

One or more embodiments described herein can include a method for identifying behavior recommendations for improving at least one of a sleep quality and a health metric of a user, the method including: presenting, by a computing system, a sleep health questionnaire at a user device, receiving, by the computing system from the user device, user input indicating at least one response to the questionnaire, determining, by the computing system and based on the user input, a proposed circadian chronotype of the user, retrieving, by the computing system from a data store, sleep patterns and behavior data for the user, determining, by the computing system, whether the retrieved sleep patterns and behavior data corresponds to the proposed circadian chronotype of the user, determining, by the computing system and based on the retrieved sleep patterns and behavior data corresponding to the proposed circadian chronotype, at least one insight, tip, or reminder for the user, the determined insight, tip, or reminder corresponding to the proposed circadian chronotype, and returning, by the computing system, at least one of (i) the proposed circadian chronotype and (ii) the insight, tip, or reminder. The method can optionally include one or more of the abovementioned features.

One or more embodiments described herein include a method including generating an output based on a user circadian chronotype. The method can optionally include one or more of the abovementioned features.

One or more embodiments described herein include a method including determining a user circadian chronotype based on user input to a questionnaire. The method can optionally include one or more of the abovementioned features.

One or more embodiments described herein include a method including determining a user circadian chronotype based on historic sleep patterns and behavior data of the user. The method can optionally include one or more of the abovementioned features.

One or more embodiments described herein include a method including determining a user circadian chronotype based on a combination of historic sleep patterns and behavior data of the user and user input to a questionnaire. The method can optionally include one or more of the abovementioned features.

One or more embodiments described herein include a method including determining a user circadian chronotype and generate output based on the determined circadian chronotype of the user. The method can optionally include one or more of the abovementioned features.

One or more embodiments described herein include a method including: receiving, by a computing system from sensors of a bed system, sensor data during a sleep session of a user, receiving, by the computing system from a user device of the user, user input indicating responses to a questionnaire, determining, by the computing system and based at least in part on the sensor data and the user input, a circadian chronotype of the user, generating, by the computing system, at least one insight, tip, or recommendation based on the circadian chronotype of the user, and transmitting, by the computing system, the at least one insight, tip, or recommendation to the user device for presentation in a GUI display of the user device. The method can optionally include one or more of the abovementioned features.

One or more embodiments described herein include a method including: retrieving, by a computing system from a data store, historic sleep patterns and behavior data a user, receiving, by the computing system from a user device of the user, user input indicating responses to a questionnaire, determining, by the computing system and based at least in part on the retrieved historic sleep patterns and behavior data and the user input, a circadian chronotype of the user, generating, by the computing system, at least one insight, tip, or recommendation based on the circadian chronotype of the user, and transmitting, by the computing system, the at least one insight, tip, or recommendation to the user device for presentation in a GUI display of the user device. The method can optionally include one or more of the abovementioned features.

The devices, system, and techniques described herein may provide one or more of the following advantages. For example, the disclosed technology can provide for selection of enhanced insights and recommendations to improve a particular user's sleep quality and/or health. The disclosed technology can leverage a robust dataset of objective sensor-derived data and system-based inferences about users, their sleep quality, sleep habits, and/or health, in combination with subjective user-perceived sleep health data, to identify and select particular insights to improve the user's sleep quality and/or health.

As another example, the disclosed technology provides for determining personalized insights for a user based on determining and/or assessing the user's circadian chronotype. Some users can be more of night owls while others may be early birds. Although the user may know if they stay up late or wake up early, they may not know how these habits correlate with their sleep quality/health. Thus, when they answer questions about their sleep habits, a computing system can process the responses to determine the user's circadian chronotype and, based on the circadian chronotype, can curate personalized insights with tips about what the user can do during a current time period (e.g., a current day) to improve their sleep over a future time period (e.g., a next day, multiple next days). Moreover, personalized insights can be generated that help the user keep their circadian rhythm on track.

The disclosed technology may be presented in user-friendly graphical user interfaces (GUIs) in such a way that gently nudges the user to take action to improve their future sleep quality and/or health and gives the user agency to improve their sleep quality and/or health. Certain actions, for example, that may have a greatest impact on improving the user's sleep quality and/or health may be progressively elevated and presented before other actions in a single GUI or across multiple GUIs presented in a mobile application at a mobile device of the user. The actions can include short term actions, such as aligning their sleep schedule for a next 7 days with a suggested schedule based on the user being a night owl, in order to build habit and help the user achieve long term sleep quality and/or health improvement. Moreover, the disclosed technology can leverage in-app notifications and prompts that gently guide users to activities that may educate them about how they can optimize their sleep and/or health over time.

The disclosed technology may also improve home automation technology. Computer technology can combine the user's subjective perception of their sleep health with automatic sensing of a user's sleep, historic sleep/health data about the user, and/or historic sleep/health data about a general population of users to generate, without need of time from expert human (e.g., doctor, therapist) for each use. This can allow for the provisioning of beneficial behavioral therapy to users who would otherwise not be able to access such information. For example, users in particularly remote or low-population-density areas may not have convenient access to behavioral therapy services, and these users can instead access recommendations that a machine generated but nevertheless specific to the particular user's life and situation. As will be understood, this can also allow the limited number of behavioral experts to provide help to many more recipients than would be possible without this technology. Instead of requiring the experts to spend time in one-on-one analysis to provide a single recipient with personalized advice, this technology can allow the experts to craft a rule-set that, when combined with a data of a particular user, generates user-specific recommendations that embody the best advice from the experts. This technology advantageously mixes both objective measures of sleep quality as well as subjective measures of sleep quality (e.g., a user-entered sleep health information). By combining both subjective and objective measures, the technology can advantageously work better than technology that uses only one or the other. For example, a user may be dealing with a physiological issue that is not (yet) reflected in objective measures (e.g., heartrate, breathing, length of sleep) but that is reflected in subjective measures (e.g., user-answered questions about their subjective feeling). For example, a user dealing with depression may initially show objective measures of very good sleep (e.g., longer sleep sessions, slower heartbeats, less gross movement), but may also as an expression of their depressive state may feel subjectively very sleepy or lethargic. This technology can advantageously consider the user's subjective feelings where technology using only objective measures may incorrectly identify the user as receiving significant high quality sleep and thus assume a high level of alertness. As will be understood, there are both clinical and sub-clinical reasons a user's objective and subjective measures of sleep may be atypical, and this technology can advantageously account for such a user.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9A is a block diagram of an example of a sensory array that can be used in a data processing system associated with a bed.

FIG. 9B is a schematic top view of a bed having an example of a sensor strip with one or more sensors that can be used in a data processing system associated with the bed.

FIG. 9C is a schematic diagram of an example bed with force sensors located at the bottom of legs of the bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19 is a conceptual diagram for determining personalized insights for a user of a bed system.

FIGS. 20A-E are example graphical user interfaces (GUIs) for presenting personalized insights to a user.

FIG. 21 is an example GUI for receiving input to a sleep health questionnaire from a user.

FIG. 22 is a block diagram of a table describing example parameters for generating personalized insights based on user input to a sleep health questionnaire.

FIGS. 23A-B is a flowchart of process for determining personalized insights for a user.

FIG. 24 is a swimlane diagram of a process for determining personalized insights for a user.

FIG. 25 is a flowchart of a process for providing personalized insights to a user in a GUI.

FIG. 26 is a flowchart of a process for automatically determining a circadian chronotype of a user.

FIG. 27 is a flowchart of another process for automatically determining a circadian chronotype of a user.

FIG. 28 is a flowchart of a process for verifying a user-perceived circadian chronotype with automated analysis of historic sleep data of a user.

Like reference symbols in the various drawings indicate like elements.

### DETAILED DESCRIPTION

The document generally describes technology for determining personalized insights for a user of a bed system based on a circadian chronotype of the user. For example, the user can provide user input at a mobile device, such as a smartphone, indicating subjective perceptions of the user's sleep quality and/or health. A computer system can process the user input to determine, based on the user's subjective responses to a sleep health questionnaire, a circadian chronotype of the user. Based on the circadian chronotype of the user, the computer system can generate a set of personalized insights that can help improve the user's sleep quality and/or health over time. The personalized insights can target improving the user's circadian chronotype, stress levels, and/or troubles falling asleep and/or staying asleep. The personalized insights can be selected based on the user's responses in their wellness profile (e.g., the sleep health questionnaire) and can include tips that may specifically address particular topics from the user's wellness profile. Such tips may be surfaced at select GUIs at the user's mobile device using randomized logic and/or machine learning rules to gently prompt the user to tack action. In some implementations, the computer system can dynamically randomize a presentation of the personalized insights at the user's mobile device such that each day (or each time the user opens a sleep-tracking application at their device), the user receives new personalized insights that are intended to gently nudge the user towards taking actions and/or building habits that improve their sleep quality and/or health and/or maintain their circadian rhythm. The computer system can also dynamically determine most relevant personalized insights to present to the user at a particular time by leveraging intelligent machine learning rules and logic.

Accordingly, the disclosed technology can provide for personalized recommendations that deepen habits of the user and give the user a sense of agency in how they change and/or improve their overall sleep quality and/or health. The disclosed technology may similarly gamify a sleep and activity experience to reinforce behaviors and/or habits that improve the user's sleep physiology. The disclosed technology can leverage robust sets of historic data about the user and other users in a general population to deliver leading indicators and information that can enable subjective and emotional response from the user to improve their overall sleep quality and/or health. The disclosed technology can also provide educational benefits with the personalized insights/recommendations to help improve various factors of sleep quality.

Example Airbed Hardware

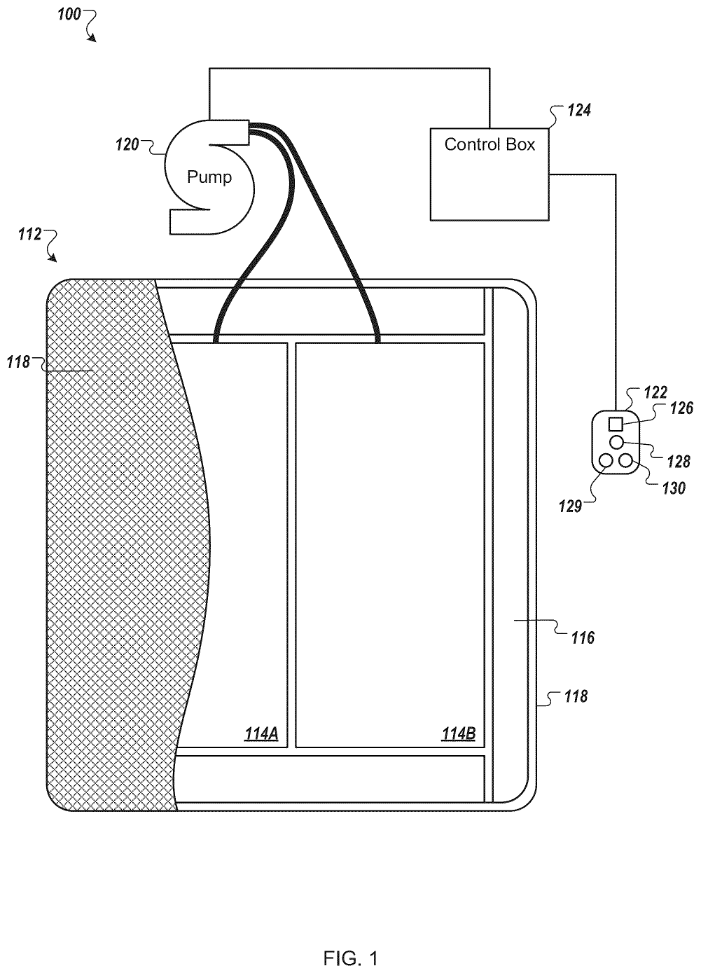

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application.

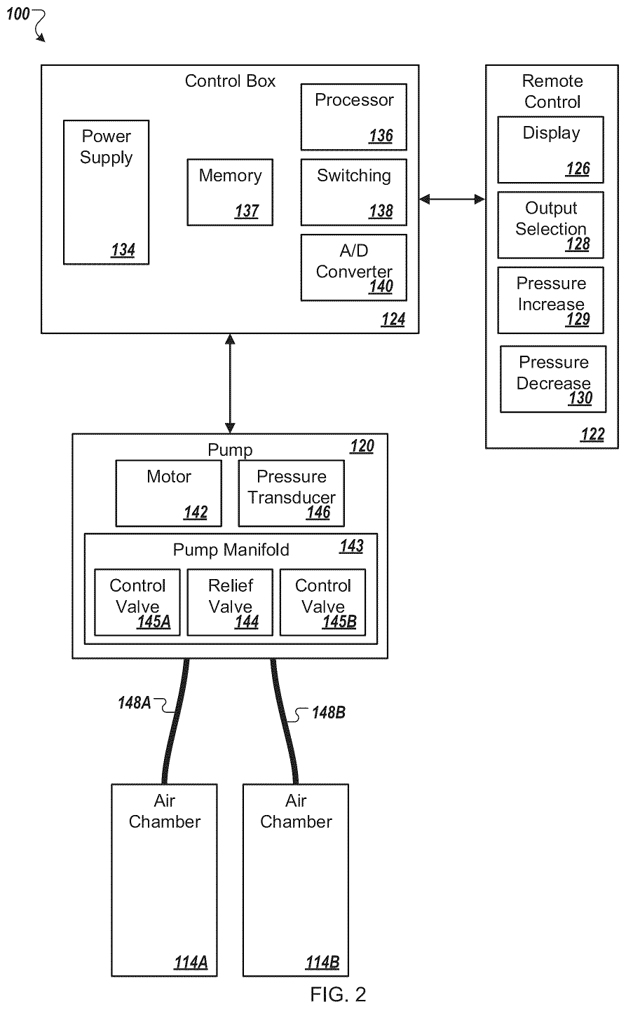

FIG. 2 is a block diagram of an example of various components of an air bed system. These components can be used in the example air bed system 100. The control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124. The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. The control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The air bed system 100 in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber. As another example, a pump can be associated with multiple chambers. A first pump can be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Additional pressure transducers can also be incorporated into the air bed system 100 such that a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 to convey the pressure information to the user. The processor 136 can also send the digital signal to other devices in wired or wireless communication with the air bed system, including but not limited to mobile devices described herein. The user can then view pressure information associated with the air bed system at their device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B to increase the chamber firmness, the pressure transducer 146 can sense pressure within the pump manifold 143. The pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126.

Generally speaking, during an inflation or deflation process, the pressure sensed within the pump manifold 143 can provide an approximation of the actual pressure within the respective air chamber that is in fluid communication with the pump manifold 143. An example method includes turning off the pump 120, allowing the pressure within the air chamber 114A or 114B and the pump manifold 143 to equalize, then sensing the pressure within the pump manifold 143 with the pressure transducer 146. Providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers. The pressure sensors can also be fluidly connected to the air chambers, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep stages) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes user motion, presence on a surface of the bed 112, weight, heart arrhythmia, snoring, partner snore, and apnea. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence can be beneficial to determine, by the processor 136, adjustment(s) to make to settings of the bed 112 (e.g., adjusting a firmness when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present. As another example, the processor 136 can determine that the user is present if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed thereon.

In some implementations, pressure fluctuations can be measured at the pump 120. For example, one or more pressure sensors can be located within one or more internal cavities of the pump 120 to detect pressure fluctuations within the pump 120. The fluctuations detected at the pump 120 can indicate pressure fluctuations in the chambers 114A and/or 114B. One or more sensors located at the pump 120 can be in fluid communication with the chambers 114A and/or 114B, and the sensors can be operative to determine pressure within the chambers 114A and/or 114B. The control box 124 can be configured to determine at least one vital sign (e.g., heartrate, respiratory rate) based on the pressure within the chamber 114A or the chamber 114B.

The control box 124 can also analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of this force input, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can receive additional biometric signals of the user from one or more other sensors or sensor arrays positioned on or otherwise integrated into the air bed system 100. For example, one or more sensors can be attached or removably attached to a top surface of the air bed system 100 and configured to detect signals such as heartrate, respiration rate, and/or motion. The processor 136 can combine biometric signals received from pressure sensors located at the pump 120, the pressure transducer 146, and/or the sensors positioned throughout the air bed system 100 to generate accurate and more precise information about the user and their sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's health information. The models can be trained using training data that includes training pressure signals and expected heartrates and/or respiratory rates. Sometimes, the control box 124 can determine user health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. The pressure signals generated by the pressure transducer 146 and received by the processor 136 can be filtered and indicated as corresponding to motion, heartbeat, or respiration. The processor 136 can attribute such fluctuations in air pressure to the user's sleep quality. Such attributions can be determined based on applying one or more machine learning models and/or algorithms to the pressure signals. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the collected data can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can be selectively controlled by the temperature controller to provide cooling or heating preferred by each user on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of a temperature controller, the pressure of the bed, the current position of the foundation or other information can be transmitted to control box 124. The control box 124 can transmit this information to the remote control 122 to be displayed to the user (e.g., on the display 126). As described above, the control box 124 can also transmit the received information to a mobile device to be displayed in a mobile application or other graphical user interface (GUI) to the user.

In some implementations, the example air bed system 100 further includes an adjustable foundation and an articulation controller configured to adjust the position of the bed 112 by adjusting the adjustable foundation supporting the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also provide different levels of massage to one or more portions of the bed 112 for one or more users. The user(s) can adjust one or more massage settings for the portions of the bed 112 using the remote control 122 and/or a mobile device in communication with the air bed system 100.

Example of a Bed in a Bedroom Environment

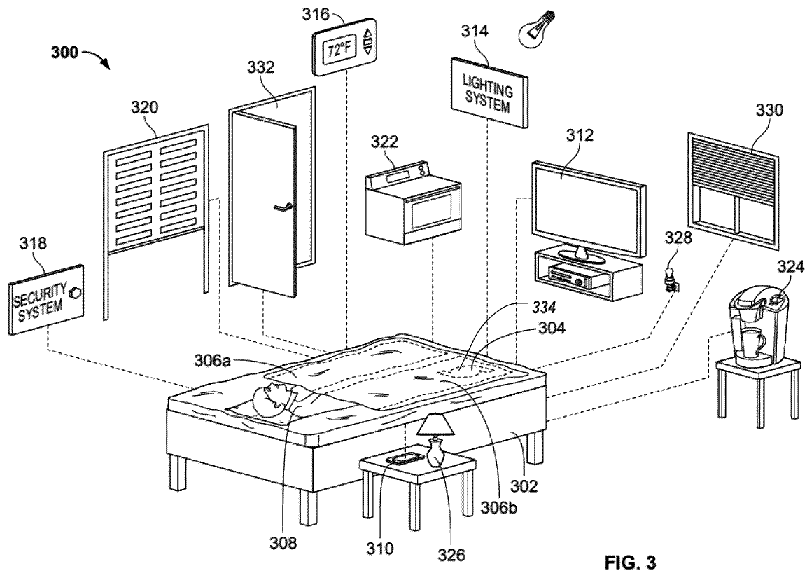

FIG. 3 shows an example environment 300 including a bed 302 in communication with devices located in and around a home. In the example shown, the bed 302 includes pump 304 for controlling air pressure within two air chambers 306a and 306b (as described above). The pump 304 additionally includes circuitry 334 for controlling inflation and deflation functionality performed by the pump 304. The circuitry 334 is programmed to detect fluctuations in air pressure of the air chambers 306a-b and use the detected fluctuations to identify bed presence of a user 308, the user's sleep state, movement, and biometric signals (e.g., heartrate, respiration rate). The detected fluctuations can also be used to detect when the user 308 is snoring and whether the user 308 has sleep apnea or other health conditions. The detected fluctuations can also be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. The control circuitry 334 located within the pump 304 can also communicate with control circuitry 334 at a remote location through a LAN or WAN (e.g., the internet). Thee control circuitry 334 can also be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality, health related) about the user 308. For example, the bed 302 can include a second pump, with each pump connected to a respective one of the air chambers 306a-b. For example, the pump 304 can be in fluid communication with the air chamber 306b to control inflation and deflation of the air chamber 306b as well as detect user signals for a user located over the air chamber 306b. The second pump can be in fluid communication with the air chamber 306a and used to control inflation and deflation of the air chamber 306a as well as detect user signals for a user located over the air chamber 306a.

As another example, the bed 302 can include one or more pressure sensitive pads or surface portions operable to detect movement, including user presence, motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located. The movement detected by the pressure sensitive pad(s) or surface portion(s) can be used by control circuitry 334 to identify user sleep state, bed presence, or biometric signals for each user. The pressure sensitive pads can also be removable rather than incorporated into the surface of the bed 302.

The bed 302 can also include one or more temperature sensors and/or array of sensors operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and falling asleep, total amount of time spent in the bed 302 for a given period of time, heartrate over a period of time, respiration rate over a period of time, or other information related to user interaction with the bed 302 by the user 308 or one or more other users. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308 so that the information presented evolves with the age of the user 308.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to accurately determine overall sleep quality and/or health of the user 308 based on information detected by components (e.g., sensors) of the bed 302.

The user 308 may also use the user device 310 as an interface for controlling air pressure of the air chambers 306a and 306b, various recline or incline positions of the bed 302, temperature of one or more surface temperature control devices of the bed 302, or for allowing the control circuitry 334 to generate control signals for other devices (as described below).

The control circuitry 334 may also communicate with other devices or systems, including but not limited to the television 312, a lighting system 314, a thermostat 316, a security system 318, home automation devices, and/or other household devices (e.g., an oven 322, a coffee maker 324, a lamp 326, a nightlight 328). Other examples of devices and/or systems include a system for controlling window blinds 330, devices for detecting or controlling states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 and other devices can occur through a network (e.g., a LAN or the Internet) or as point-to-point communication (e.g., BLUETOOTH, radio communication, or a wired connection). Control circuitry 334 of different beds 302 can also communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. The control signals can be generated based on information collected by the control circuitry 334, including information related to user interaction with the bed 302 by the user 308 and/or one or more other users. Information collected from other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for other devices, the control circuitry 334 can provide collected information (e.g., information related to user movement, bed presence, sleep state, or biometric signals) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and other devices in the house. The central controller can include sensors that detect signals that can be used by the control circuitry 334 and/or the central controller to determine information about the user 308 (e.g., biometric or other health data, sleep quality). The sensors can detect signals including such as ambient light, temperature, humidity, volatile organic compound(s), pulse, motion, and audio. These signals can be combined with signals detected by sensors of the bed 302 to determine accurate information about the user 308's health and sleep quality. The central controller can provide controls (e.g., user-defined, presets, automated, user initiated) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that outputs information and receives user input. The display can output information such as the user 308's health, sleep quality, weather, security integration features, lighting integration features, heating and cooling integration features, and other controls to automate devices in the house. The central controller can operate to provide the user 308 with functionality and control of multiple different types of devices in the house as well as the user 308's bed 302.

As an illustrative example of FIG. 3, the control circuitry 334 integrated with the pump 304 can detect a feature of a mattress of the bed 302, such as an increase in pressure in the air chamber 306b, and use this detected increase to determine that the user 308 is present on the bed 302. The control circuitry 334 may also identify a heartrate or respiratory rate for the user 308 to identify that the increased pressure is due to a person sitting, laying, or resting on the bed 302, rather than an inanimate object (e.g., a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experience improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 may use collected information (including information related to user interaction with the bed 302 by the user 308, environmental information, time information, and user input) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 pm and 11:00 pm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

Given the above example user bed presence, sleep, and wake patterns for the user 308, if the user 308 is detected as being on the bed 302 at 3:00 pm, the control circuitry 334 can determine that the user 308's presence on the bed 302 is temporary, and use this determination to generate different control signals than if the control circuitry 334 determined the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 got out of bed at 3:00 am, the control circuitry 334 can use identified patterns for the user 308 to determine the user has gotten up temporarily (e.g., to use the bathroom, get a glass of water). The control circuitry 334 can turn on underbed lighting to assist the user 308 in carefully moving around the bed 302 and room. By contrast, if the control circuitry 334 identifies that the user 308 got out of the bed 302 at 6:40 am, the control circuitry 334 can determine the user 308 is up for the day and generate a different set of control signals (e.g., the control circuitry 334 can turn on light 326 near the bed 302 and/or raise the window blinds 330). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

The bed 302 can also generate control signals based on communication with one or more devices. As an illustrative example, the control circuitry 334 can receive an indication from the television 312 that the television 312 is turned on. If the television 312 is located in a different room than the bed 302, the control circuitry 334 can generate a control signal to turn the television 312 off upon making a determination that the user 308 has gone to bed for the evening or otherwise is remaining in the room with the bed 302. If presence of the user 308 is detected on the bed 302 during a particular time range (e.g., between 8:00 pm and 7:00 am) and persists for longer than a threshold period of time (e.g., 10 minutes), the control circuitry 334 can determine the user 308 is in bed for the evening. If the television 312 is on, as described above, the control circuitry 334 can generate a control signal to turn the television 312 off. The control signals can be transmitted to the television (e.g., through a directed communication link or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 indicated a preference for watching morning news upon getting out of bed). The control circuitry 334 can accordingly generate and transmit the control signal to the television 312 (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening and generate control signals to cause lights in one or more rooms other than the room in which the bed 302 is located to switch off. The control circuitry 334 can generate and transmit control signals to turn off lights in all common rooms, but not in other bedrooms. As another example, the control signals can indicate that lights in all rooms other than the room in which the bed 302 is located are to be turned off, while one or more lights located outside of the house containing the bed 302 are to be turned on. The control circuitry 334 can generate and transmit control signals to cause the nightlight 328 to turn on in response to determining user 308 bed presence or that the user 308 is asleep. The control circuitry 334 can also generate first control signals for turning off a first set of lights (e.g., lights in common rooms) in response to detecting user bed presence, and second control signals for turning off a second set of lights (e.g., lights in the room where the bed 302 is located) when detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also implement a sunrise lighting scheme when the user 308 wakes up in the morning. The control circuitry 334 can determine that the user 308 is awake for the day, for example, by detecting that the user 308 has gotten off the bed 302 (e.g., is no longer present on the bed 302) during a specified time frame (e.g., between 6:00 am and 8:00 am). The control circuitry 334 can also monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. The sunrise lighting scheme can include lighting the bedroom with blue light to gently assist the user 308 in waking up and becoming active.

The control circuitry 334 may also generate different control signals for controlling actions of components depending on a time of day that user interactions with the bed 302 are detected. For example, the control circuitry 334 can use historical user interaction information to determine that the user 308 usually falls asleep between 10:00 pm and 11:00 pm and usually wakes up between 6:30 am and 7:30 am on weekdays. The control circuitry 334 can use this information to generate a first set of control signals for controlling the lighting system 314 if the user 308 is detected as getting out of bed at 3:00 am (e.g., turn on lights that guide the user 308 to a bathroom or kitchen) and to generate a second set of control signals for controlling the lighting system 314 if the user 308 is detected as getting out of bed after 6:30 am.

In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach their destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). Buffer time may be added to these timeframes. For example, if the user is identified as typically going to bed between 10:00 pm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 pm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of year (e.g., earlier bed time during winter vs. summer) or at different times of the week (e.g., user 308 wakes up earlier on weekdays than on weekends).

The control circuitry 334 can distinguish between the user 308 going to bed for an extended period (e.g., for the night) as opposed to being present on the bed 302 for a shorter period (e.g., for a nap) by sensing duration of presence of the user 308 (e.g., by detecting pressure and/or temperature signals of the user 308 on the bed 302 by sensors integrated into the bed 302). In some examples, the control circuitry 334 can distinguish between the user 308 going to bed for an extended period (e.g., for the night) versus going to bed for a shorter period (e.g., for a nap) by sensing duration of the user 308's sleep. The control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

The control circuitry 334 can automatically determine the bed time range of the user 308 without requiring user inputs. The control circuitry 334 may also determine the bed time range automatically and in combination with user inputs (e.g., using signals sensed by sensors of the bed 302 and/or the central controller). The control circuitry 334 can set the bed time range directly according to user inputs. The control circuitry 334 can associate different bed times with different days of the week. In each of these examples, the control circuitry 334 can control components (e.g., the lighting system 314, thermostat 316, security system 318, oven 322, coffee maker 324, lamp 326, nightlight 328), as a function of sensed bed presence and the bed time range.

The control circuitry 334 can also determine control signals to be transmitted to the thermostat 316 based on user-inputted preferences and/or maintaining improved or preferred sleep quality of the user 308. For example, the control circuitry 334 can determine, based on historic sleep patterns and quality of the user 308 and by applying machine learning models, that the user 308 experiences their best sleep when the bedroom is at 74 degrees. The control circuitry 334 can receive temperature signals from devices and/or sensors in the bedroom indicating a bedroom temperature. When the temperature is below 74 degrees, the control circuitry 334 can determine control signals that cause the thermostat 316 to activate a heating unit to raise the temperature to 74 degrees in the bedroom. When the temperature is above 74 degrees, the control circuitry 334 can determine control signals that cause the thermostat 316 to activate a cooling unit to lower the temperature back to 74 degrees. Sometimes, the control circuitry 334 can determine control signals that cause the thermostat 316 to maintain the bedroom within a temperature range intended to keep the user 308 in particular sleep states and/or transition to next preferred sleep states.

Similarly, the control circuitry 334 can generate control signals to cause heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. The user 308 can pre-program various times at which the temperature at the bed surface should be raised or lowered. As another example, temperature sensors on the bed surface can detect microclimates of the user 308. When a detected microclimate drops below a predetermined threshold temperature, the control circuitry 334 can activate a heating element to raise the user 308's body temperature, thereby improving the user 308's comfortability, maintaining their sleep cycle, transitioning the user 308 to a next preferred sleep state, and/or maintaining or improving the user 308's sleep quality.

In response to detecting user bed presence and/or that the user 308 is asleep, the control circuitry 334 can also cause the thermostat 316 to change the temperature in different rooms to different values. Other control signals are also possible, and can be based on user preference and user input. Moreover, the control circuitry 334 can receive temperature information from the thermostat 316 and use this information to control functions of the bed 302 or other devices (e.g., adjusting temperatures of heating elements of the bed 302, such as a foot warming pad). The control circuitry 334 may also generate and transmit control signals for controlling other temperature control systems, such as floor heating elements in the bedroom or other rooms.

The control circuitry 334 can communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. As another example, the control circuitry 334 can generate and transmit control signals to cause the security system 318 to disable in response to determining that the user 308 is awake for the day (e.g., user 308 is no longer present on the bed 302).

The control circuitry 334 can also receive alerts from the security system 318 and indicate the alert to the user 308. For example, the security system can detect a security breach (e.g., someone opened the door 332 without entering the security code, someone opened a window when the security system 318 is engaged) and communicate the security breach to the control circuitry 334. The control circuitry 334 can then generate control signals to alert the user 308, such as causing the bed 302 to vibrate, causing portions of the bed 302 to articulate (e.g., the head section to raise or lower), causing the lamp 326 to flash on and off at regular intervals, etc. The control circuitry 334 can also alert the user 308 of one bed 302 about a security breach in another bedroom, such as an open window in a kid's bedroom. The control circuitry 334 can send an alert to a garage door controller (e.g., to close and lock the door). The control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller, which can then determine one or more responses to the security breach.

The control circuitry 334 can additionally generate and transmit control signals for controlling the garage door 320 and receive information indicating a state of the garage door 320 (e.g., open or closed). The control circuitry 334 can also request information on a current state of the garage door 320. If the control circuitry 334 receives a response (e.g., from the garage door opener) that the garage door 320 is open, the control circuitry 334 can notify the user 308 that the garage door is open (e.g., by displaying a notification or other message at the user device 310, outputting a notification at the central controller), and/or generate a control signal to cause the garage door opener to close the door. The control circuitry 334 can also cause the bed 302 to vibrate, cause the lighting system 314 to flash lights in the bedroom, etc. Control signals can also vary depend on the age of the user 308. Similarly, the control circuitry 334 can similarly send and receive communications for controlling or receiving state information associated with the door 332 or the oven 322.

In some implementations, different alerts can be generated for different events. For example, the control circuitry 334 can cause the lamp 326 (or other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., a child has gotten out of bed in the middle of the night as sensed by a sensor in the child's bed). Other examples of alerts include a smoke detector detecting smoke (and communicating this detection to the control circuitry 334), a carbon monoxide tester, a heater malfunctioning, or an alert from another device capable of communicating with the control circuitry 334 and detecting an occurrence to bring to the user 308's attention.

The control circuitry 334 can also communicate with a system or device for controlling a state of the window blinds 330. For example, in response to determining that the user 308 is up for the day or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. The control circuitry 334 can also generate and transmit control signals that cause a first set of blinds to close in response to detecting user bed presence and a second set of blinds to close in response to detecting that the user 308 is asleep.

As other examples, in response to determining that the user 308 is awake for the day, the control circuitry 334 can generate and transmit control signals to the coffee maker 324 to cause the coffee maker 324 to brew coffee. The control circuitry 334 can generate and transmit control signals to the oven 322 to cause the oven 322 to begin preheating. The control circuitry 334 can use information indicating that the user 308 is awake for the day along with information indicating that the time of year is currently winter and/or that the outside temperature is below a threshold value to generate and transmit control signals to cause a car engine block heater to turn on. The control circuitry 334 can generate and transmit control signals to cause devices to enter a sleep mode in response to detecting user bed presence, or in response to detecting that the user 308 is asleep (e.g., causing a mobile phone of the user 308 to switch into sleep or night mode so that notifications are muted to not disturb the user 308's sleep). Later, upon determining that the user 308 is up for the day, the control circuitry 334 can generate and transmit control signals to cause the mobile phone to switch out of sleep/night mode.

The control circuitry 334 can also communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302), the control circuitry 334 can generate and transmit control signals to cause noise cancelation devices to activate. The noise cancelation devices can be part of the bed 302 or located in the bedroom. Upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions. For example, the articulation controller can adjust the bed 302 from a flat position to a position in which a head portion of a mattress of the bed 302 is inclined upward (e.g., to facilitate a user sitting up in bed, reading, and/or watching television). Sometimes, the bed 302 includes multiple separately articulable sections. Portions of the bed corresponding to the locations of the air chambers 306a and 306b can be articulated independently from each other, to allow one person to rest in a first position (e.g., a flat position) while a second person rests in a second position (e.g., a reclining position with the head raised at an angle from the waist). Separate positions can be set for two different beds (e.g., two twin beds placed next to each other). The foundation of the bed 302 can include more than one zone that can be independently adjusted. The articulation controller can also provide different levels of massage to one or more users on the bed 302 or cause the bed to vibrate to communicate alerts to the user 308 as described above.

The control circuitry 334 can adjust positions (e.g., incline and decline positions for the user 308 and/or an additional user) in response to user interactions with the bed 302 (e.g., causing the articulation controller to adjust to a first recline position in response to sensing user bed presence). The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the bed position to a preferred user sleeping position (e.g., due to the user turning off the television 312 while the user 308 is in bed indicating the user 308 wishes to go to sleep).

In some implementations, the control circuitry 334 can control the articulation controller to wake up one user without waking another user of the bed 302. For example, the user 308 and a second user can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for devices until both the user 308 and a second user are detected in the bed 302. The control circuitry 334 can generate a first set of control signals to cause the lighting system 314 to turn off a first set of lights upon detecting bed presence of the user 308 and generate a second set of control signals for turning off a second set of lights in response to detecting bed presence of a second user. The control circuitry 334 can also wait until it has been determined that both users are awake for the day before generating control signals to open the window blinds 330. One or more other home automation control signals can be determined and generated by the control circuitry 334, the user device 310, and/or the central controller.

Examples of Data Processing Systems Associated with a Bed

Described are example systems and components for data processing tasks that are, for example, associated with a bed. In some cases, multiple examples of a particular component or group of components are presented. Some examples are redundant and/or mutually exclusive alternatives. Connections between components are shown as examples to illustrate possible network configurations for allowing communication between components. Different formats of connections can be used as technically needed/desired. The connections generally indicate a logical connection that can be created with any technologically feasible format. For example, a network on a motherboard can be created with a printed circuit board, wireless data connections, and/or other types of network connections. Some logical connections are not shown for clarity (e.g., connections with power supplies and/or computer readable memory).

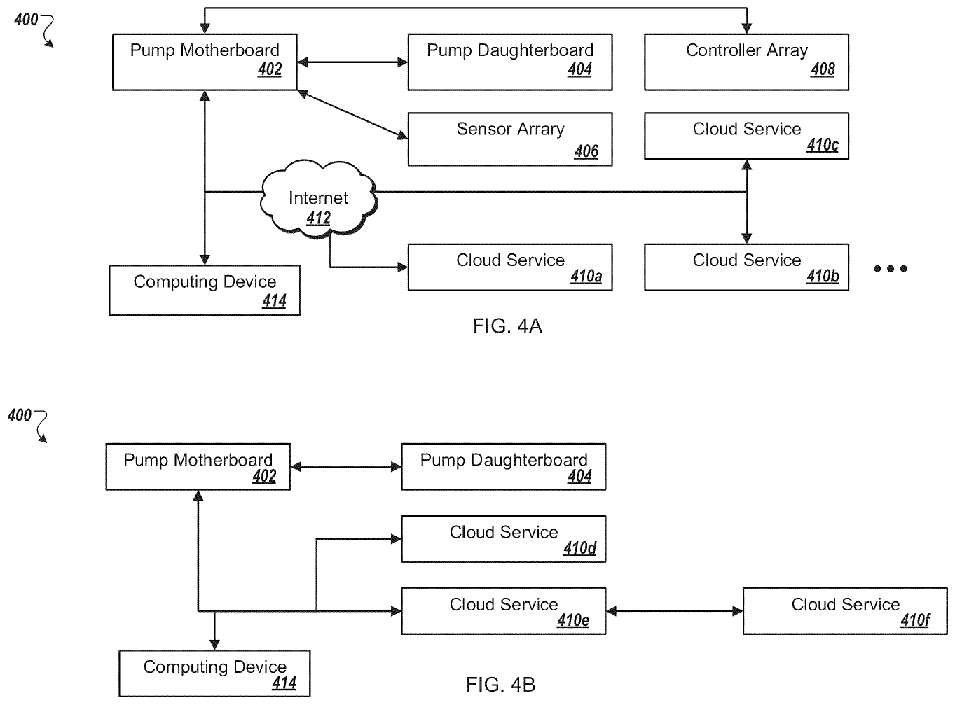

FIG. 4A is a block diagram of an example data processing system 400 that can be associated with a bed system, including those described above (e.g., see FIGS. 1-3). The system 400 includes a pump motherboard 402 and a pump daughterboard 404. The system 400 includes a sensor array 406 having one or more sensors configured to sense physical phenomenon of the environment and/or bed, and to report sensing back to the pump motherboard 402 (e.g., for analysis). The sensor array 406 can include one or more different types of sensors, including but not limited to pressure, temperature, light, movement (e.g. motion), and audio. The system 400 also includes a controller array 408 that can include one or more controllers configured to control logic-controlled devices of the bed and/or environment (e.g., home automation devices, security systems light systems, and other devices described in FIG. 3). The pump motherboard 400 can be in communication with computing devices 414 and cloud services 410 over local networks (e.g., Internet 412) or otherwise as is technically appropriate.

In FIG. 4A, the pump motherboard 402 and daughterboard 404 are communicably coupled. They can be conceptually described as a center or hub of the system 400, with the other components conceptually described as spokes of the system 400. This can mean that each spoke component communicates primarily or exclusively with the pump motherboard 402. For example, a sensor of the sensor array 406 may not be configured to, or may not be able to, communicate directly with a corresponding controller. Instead, the sensor can report a sensor reading to the motherboard 402, and the motherboard 402 can determine that, in response, a controller of the controller array 408 should adjust some parameters of a logic controlled device or otherwise modify a state of one or more peripheral devices.

One advantage of a hub-and-spoke network configuration, or a star-shaped network, is a reduction in network traffic compared to, for example, a mesh network with dynamic routing. If a particular sensor generates a large, continuous stream of traffic, that traffic is transmitted over one spoke to the motherboard 402. The motherboard 402 can marshal and condense that data to a smaller data format for retransmission for storage in a cloud service 410. Additionally or alternatively, the motherboard 402 can generate a single, small, command message to be sent down a different spoke in response to the large stream. For example, if the large stream of data is a pressure reading transmitted from the sensor array 406 a few times a second, the motherboard 402 can respond with a single command message to the controller array 408 to increase the pressure in an air chamber of the bed. In this case, the single command message can be orders of magnitude smaller than the stream of pressure readings.

As another advantage, a hub-and-spoke network configuration can allow for an extensible network that accommodates components being added, removed, failing, etc. This can allow more, fewer, or different sensors in the sensor array 406, controllers in the controller array 408, computing devices 414, and/or cloud services 410. For example, if a particular sensor fails or is deprecated by a newer version, the system 400 can be configured such that only the motherboard 402 needs to be updated about the replacement sensor. This can allow product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. For each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component are discussed. Sometimes, two or more components of the system 400 can be realized in a single alternative component; some components can be realized in multiple, separate components; and/or some functionality can be provided by different components.

FIG. 4B is a block diagram showing communication paths of the system 400. As described, the motherboard 402 and daughterboard 404 may act as a hub of the system 400. When the pump daughterboard 404 communicates with cloud services 410 or other components, communications may be routed through the motherboard 402. This may allow the bed to have a single connection with the Internet 412. The computing device 414 may also have a connection to the Internet 412, possibly through the same gateway used by the bed and/or a different gateway (e.g., a cell service provider).

In FIG. 4B, cloud services 410d and 410e may be configured such that the motherboard 402 communicates with the cloud service directly (e.g., without having to use another cloud service 410 as an intermediary). Additionally or alternatively, some cloud services 410 (e.g., 410f) may only be reachable by the motherboard 402 through an intermediary cloud service (e.g., 410e). While not shown here, some cloud services 410 may be reachable either directly or indirectly by the pump motherboard 402.

Additionally, some or all of the cloud services 410 may communicate with other cloud services, including the transfer of data and/or remote function calls according to any technologically appropriate format. For example, one cloud service 410 may request a copy for another cloud service's 410 data (e.g., for purposes of backup, coordination, migration, calculations, data mining). Many cloud services 410 may also contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

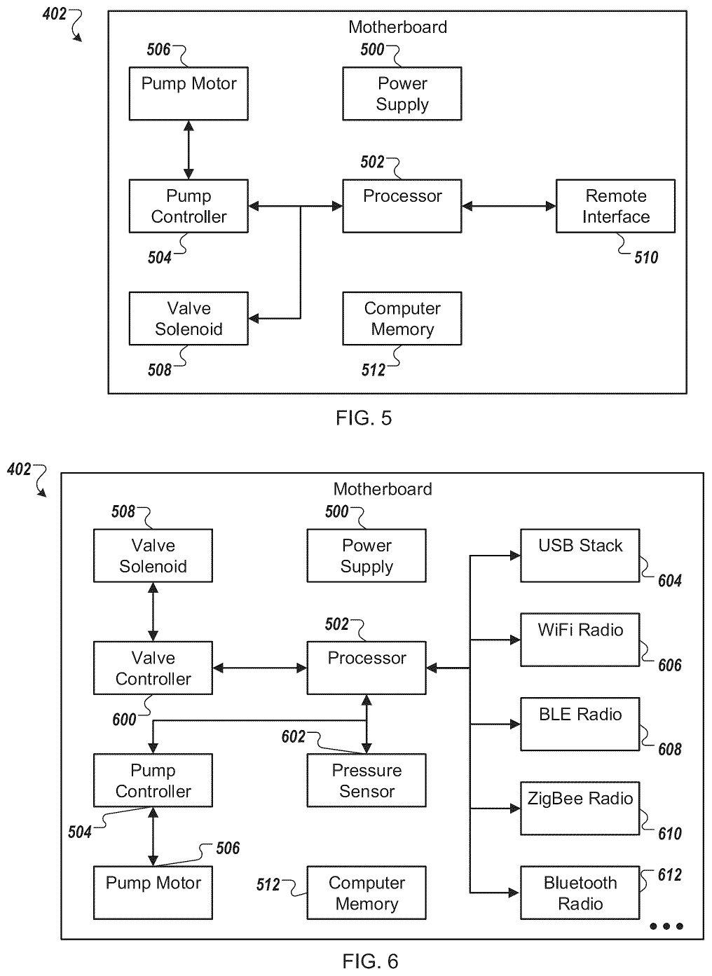

FIG. 5 is a block diagram of an example motherboard 402 in a data processing system associated with a bed system (e.g., refer to FIGS. 1-3). In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply may include a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware.

The memory 512 is generally one or more devices for storing data, which may include long term stable data storage (e.g., on a hard disk), short term unstable (e.g., on Random Access Memory), or any other technologically appropriate configuration.

The motherboard 402 includes a pump controller 504 and a pump motor 506. The pump controller 504 can receive commands from the processor 502 to control functioning of the pump motor 506. For example, the pump controller 504 can receive a command to increase pressure of an air chamber by 0.3 pounds per square inch (PSI). The pump controller 504, in response, engages a valve so that the pump motor 506 pumps air into the selected air chamber, and can engage the pump motor 506 for a length of time that corresponds to 0.3 PSI or until a sensor indicates that pressure has been increased by 0.3 PSI. Sometimes, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

FIG. 6 is a block diagram of another example motherboard 402. Compared to the motherboard 402 in FIG. 5, the motherboard 402 in FIG. 6 can contain more components and provide more functionality in some applications.

This motherboard 402 can further include a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

The valve controller 600 can convert commands from the processor 502 into control signals for the valve solenoid 508. For example, the processor 502 can issue a command to the valve controller 600 to connect the pump to a particular air chamber out of a group of air chambers in an air bed. The valve controller 600 can control the position of the valve solenoid 508 so the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any devices, including but not limited to peripheral sensors, peripheral controllers, computing devices, and devices and services connected to the Internet 412.

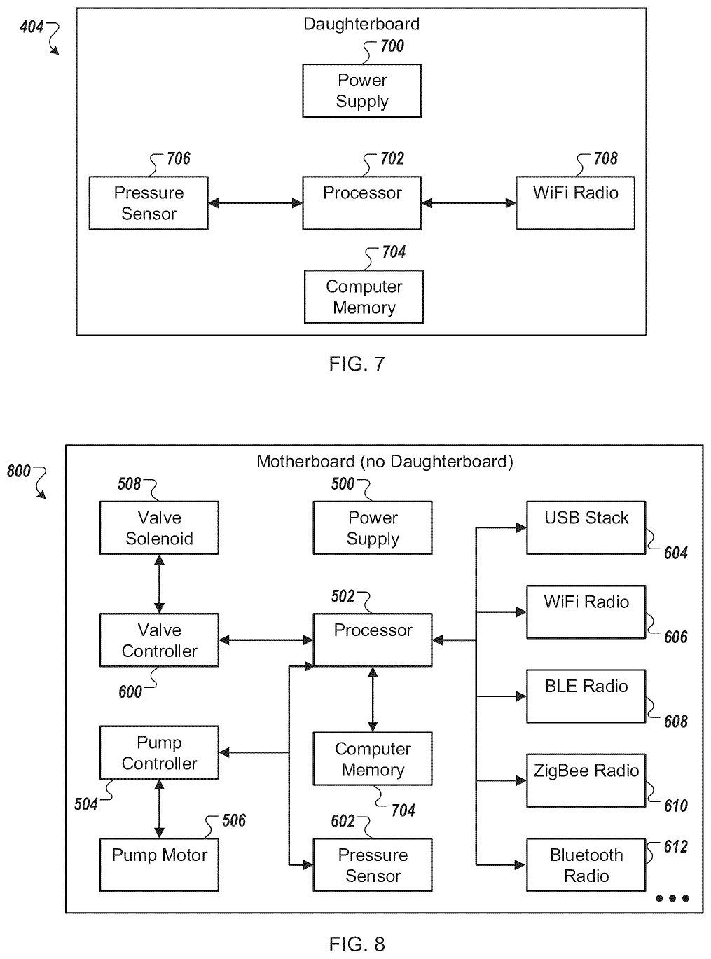

FIG. 7 is a block diagram of an example daughterboard 404 used in a data processing system associated with a bed system described herein. One or more daughterboards 404 can be connected to the motherboard 402. Some daughterboards 404 can be designed to offload particular and/or compartmentalized tasks from the motherboard 402. This can be advantageous if the particular tasks are computationally intensive, proprietary, or subject to future revisions. For example, the daughterboard 404 can be used to calculate a particular sleep data metric. This metric can be computationally intensive, and calculating the metric on the daughterboard 404 can free up resources of the motherboard 402 while the metric is calculated. The sleep metric may be subject to future revisions. To update the system 400 with the new metric, it is possible that only the daughterboard 404 calculates the metric to be replaced. In this case, the same motherboard 402 and other components can be used, saving the need to perform unit testing of additional components instead of just the daughterboard 404.

The daughterboard 404 includes a power supply 700, a processor 702, computer readable memory 704, a pressure sensor 706, and a WiFi radio 708. The processor 702 can use the pressure sensor 706 to gather information about pressure of air bed chambers. The processor 702 can perform an algorithm to calculate a sleep metric (e.g., sleep quality, bed presence, whether the user fell asleep, a heartrate, a respiration rate, movement, etc.). Sometimes, the sleep metric can be calculated from only air chamber pressure. The sleep metric can also be calculated using signals from a variety of sensors (e.g., movement, pressure, temperature, and/or audio sensors). The processor 702 can receive that data from sensors that may be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can generate instructions for outputting the sleep metric to the user or using the sleep metric to determine other user information or controls to control the bed and/or peripheral devices.

FIG. 8 is a block diagram of an example motherboard 800 with no daughterboard used in a data processing system associated with a bed system. In this example, the motherboard 800 can perform most, all, or more of the features described with reference to the motherboard 402 in FIG. 6 and the daughterboard 404 in FIG. 7.

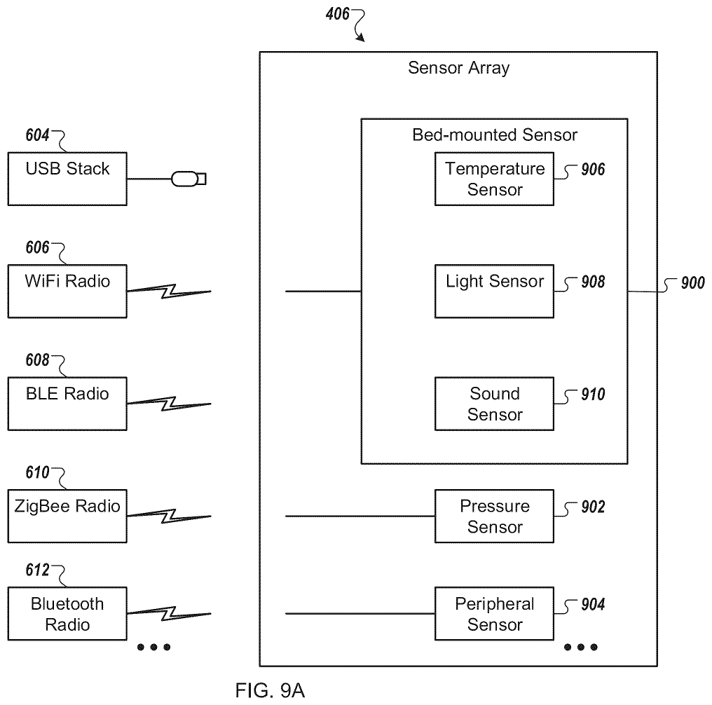

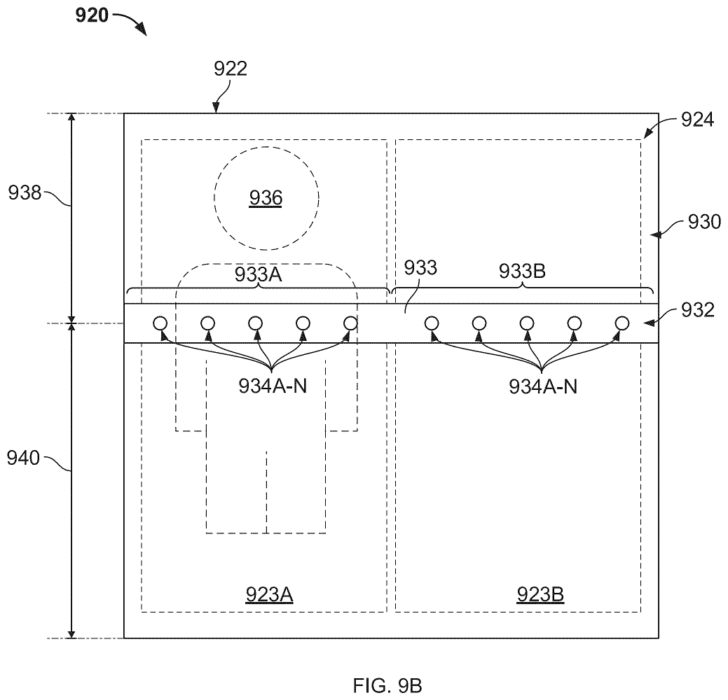

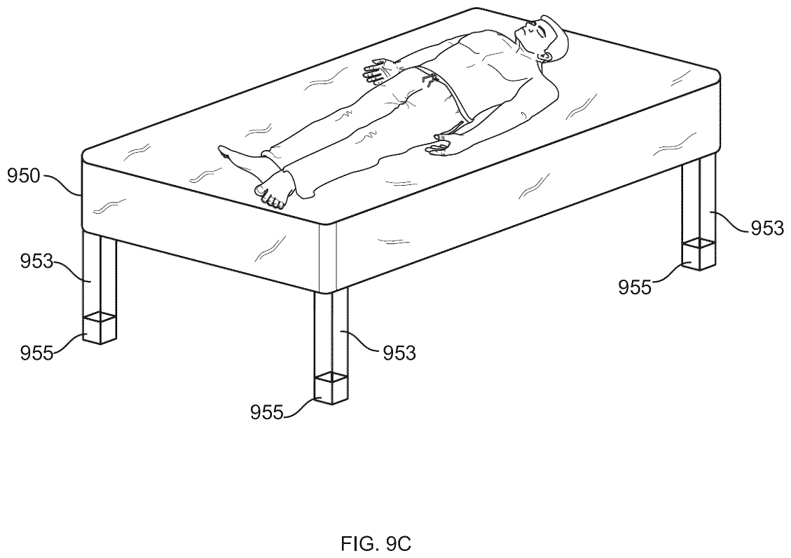

FIG. 9A is a block diagram of an example sensory array 406 used in a data processing system associated with a bed system described herein. The sensor array 406 is a conceptual grouping of some or all peripheral sensors that communicate with the motherboard 402 but are not native to the motherboard 402. The peripheral sensors 902, 904, 906, 908, 910, etc. of the sensor array 406 communicate with the motherboard 402 through one or more network interfaces 604, 606, 608, 610, and 612 of the motherboard, as is appropriate for the configuration of the particular sensor. For example, a sensor that outputs a reading over a USB cable can communicate through the USB stack 604.

Some peripheral sensors of the sensor array 406 can be bed mounted sensors 900 (e.g., temperature sensor 906, light sensor 908, sound sensor 910). The bed mounted sensors 900 can be embedded into a bed structure and sold with the bed, or later affixed to the structure (e.g., part of a pressure sensing pad that is removably installed on a top surface of the bed, part of a temperature sensing or heating pad that is removably installed on the top surface of the bed, integrated into the top surface, attached along connecting tubes between a pump and air chambers, within air chambers, attached to a headboard, attached to one or more regions of an adjustable foundation). One or more of the sensors 902 can be load cells or force sensors as described in FIG. 9C. Other sensors 902 and 904 may not be mounted to the bed and can include a pressure sensor 902 and/or peripheral sensor 904. For example, the sensors 902 and 904 can be integrated or otherwise part of a user mobile device (e.g., mobile phone, wearable device). The sensors 902 and 904 can also be part of a central controller for controlling the bed and peripheral devices. Sometimes, the sensors 902 and 904 can be part of one or more home automation devices or other peripheral devices.

Sometimes, some or all of the bed mounted sensors 900 and/or sensors 902 and 904 share networking hardware (e.g., a conduit that contains wires from each sensor, a multi-wire cable or plug that, when affixed to the motherboard 402, connect all the associated sensors with the motherboard 402). One, some, or all the sensors 902, 904, 906, 908, and 910 can sense features of a mattress (e.g., pressure, temperature, light, sound, and/or other features) and features external to the mattress. Sometimes, pressure sensor 902 can sense pressure of the mattress while some or all the sensors 902, 904, 906, 908, and 910 sense features of the mattress and/or features external to the mattress.

FIG. 9B is a schematic top view of a bed 920 having a sensor strip 932 with sensors 934A-N used in a data processing system associated with the bed 920. The bed 920 includes a mattress 922 (e.g., refer to FIG. 1). The mattress 922 can have a foam tub 930 beneath a top of the mattress 922. The foam tub 930 can have air chamber 923A and/or 923B, similar to those described herein.

The sensor strip 932 can be attached across the mattress top 924 from one lateral side to an opposing lateral side (e.g., from left to right). The sensor strip 932 can be attached proximate to a head section of the mattress 922 to measure temperature and/or humidity values around a chest area of a user 936. The sensor strip 932 can also be placed at a center point (e.g., midpoint) of the mattress 922 such that the distances 938 and 940 are equal to each other. The sensor strip 932 can be placed at other locations to capture temperature and/or humidity values at the top of the mattress 922.

The sensors 934A-N can be any one or more of the temperature sensors 906 described in FIG. 9A. The sensor strip 932 can also include a carrier strip 933 having a first strip portion 933A and a second strip portion 933B. The carrier strip 933 can be releasably attached to the foam tub layer 920 and extend between the opposite lateral ends of the foam tub 920. The sensor strip 932 can have first sensors 934A-N and second sensors 934A-N. Each of the first and second sensors 934A-N can have five sensors each. For example, a sensor strip 932 for a king or queen size mattress can have a total of ten sensors. When the user 936 is positioned on top of the mattress 922 over the air chamber 923A, the first sensors 934A-N can measure temperature and/or humidity of the mattress top 924 above the air chamber 923A. Those values can be used to, for example, determine a conditioned airflow to supply to the air chamber 923A. Temperature and/or humidity values measured by the second sensors 934A-N can be used to, for example, determine a conditioned airflow to supply to the air chamber 923B. The bed system 920 can provide for custom airflow to different portions of the mattress 922 based on body temperatures of users and/or temperatures of different portions of the mattress top 924.

Sometimes, two separate sensor strips can be attached to the mattress 922 (e.g., a first sensor strip over the air chamber 923A and a second sensor strip, separate from the first sensor strip, over the air chamber 923B). The first and second sensor strips can be attached to a center of the mattress top 924 via fastening elements, such as adhesive. The sensor strip 932 can also be easily replaced with another sensor strip.

FIG. 9C is a schematic diagram of an example bed with force sensors 955 located at the bottom of legs 953 of the bed (e.g., in four, six, eight, or another number of legs). The force sensors 955 may also be located elsewhere on the bed with similar effect (e.g., between the legs 953 and platform 950). When a strain gauge is used as the force sensors 955, the force sensor(s) 955 can be positioned nearer centers of the legs 953. The force sensors 955 can be load cells.

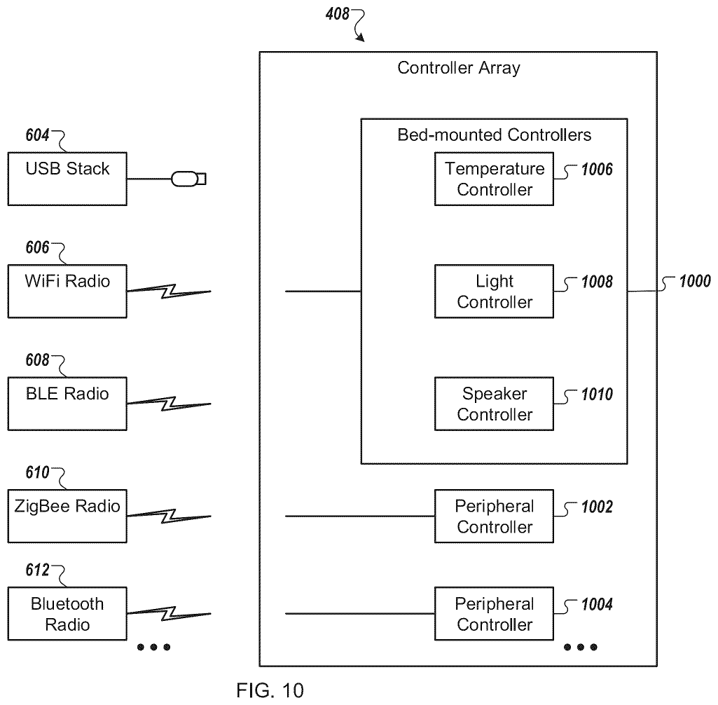

FIG. 10 is a block diagram of an example controller array 408 used in a data processing system associated with a bed system. The controller array 408 is a conceptual grouping of some or all peripheral controllers that communicate with the motherboard 402 but are not native to the motherboard 402. The peripheral controllers can communicate with the motherboard 402 through one or more of the network interfaces 604, 606, 608, 610, and 612 of the motherboard, as is appropriate for the configuration of the particular controller. Some of the controllers can be bed mounted controllers 1000, such as a temperature controller 1006, a light controller 1008, and a speaker controller 1010, as described in reference to bed-mounted sensors in FIG. 9A. Peripheral controllers 1002 and 1004 can be in communication with the motherboard 402, but optionally not mounted to the bed.

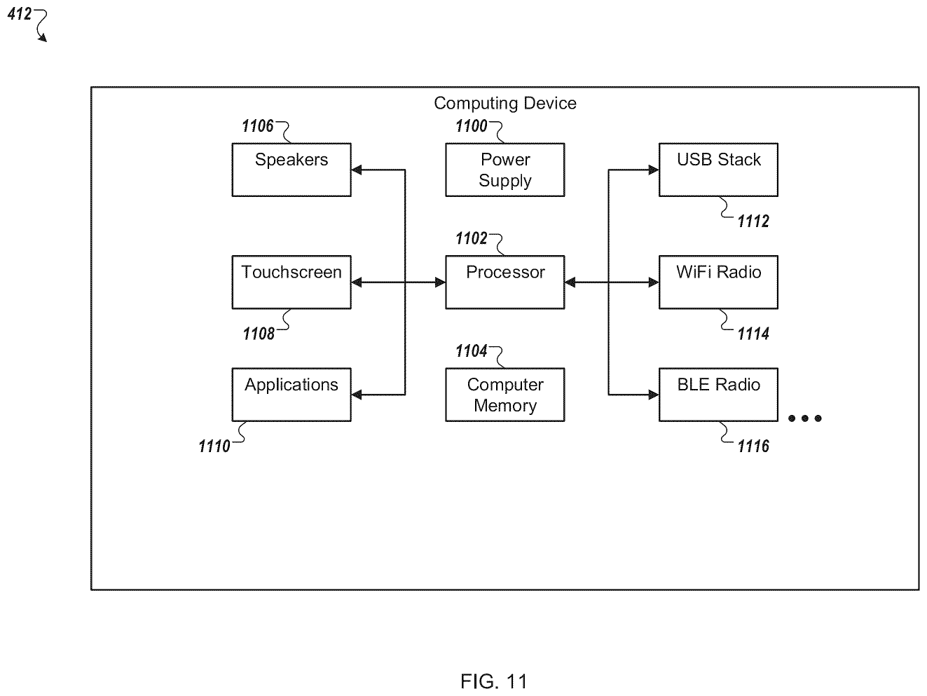

FIG. 11 is a block diagram of an example computing device 412 used in a data processing system associated with a bed system. The computing device 412 can include computing devices used by a user of a bed including but not limited to mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by speakers 1106, a touchscreen 1108, or other not shown components (e.g., a pointing device or keyboard). The computing device 412 can run applications 1110 including, for example, applications to allow the user to interact with the system 400. These applications can allow a user to view information about the bed (e.g., sensor readings, sleep metrics), information about themselves (e.g., health conditions detected based on signals sensed at the bed), and/or configure the system 400 behavior (e.g., set desired firmness, set desired behavior for peripheral devices). The computing device 412 can be used in addition to, or to replace, the remote control 122 described above.

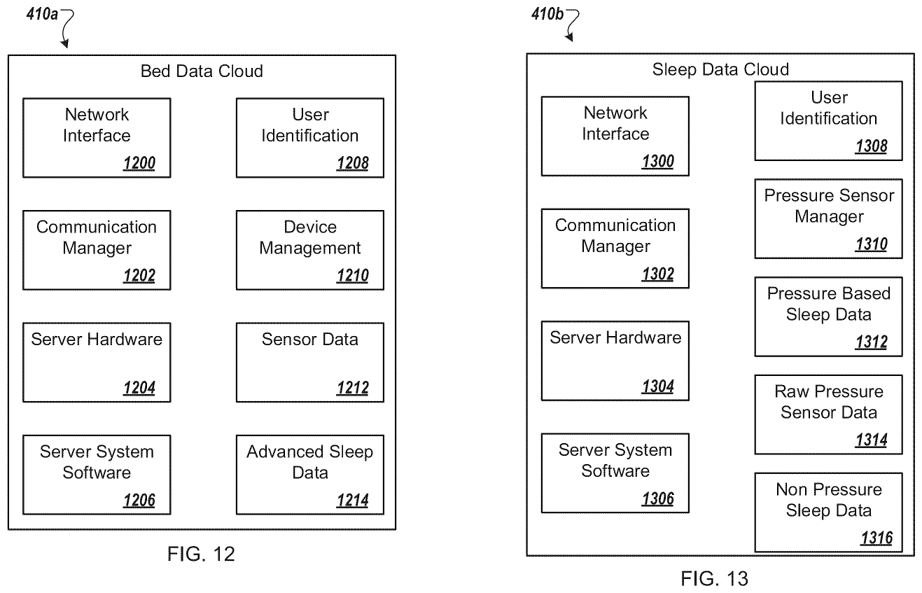

FIG. 12 is a block diagram of an example bed data cloud service 410a used in a data processing system associated with a bed system. Here, the bed data cloud service 410a is configured to collect sensor data and sleep data from a particular bed, and to match the data with one or more users that used the bed when the data was generated.

The bed data cloud service 410a includes a network interface 1200, a communication manager 1202, server hardware 1204, and server system software 1206. The bed data cloud service 410a is also shown with a user identification module 1208, a device management 1210 module, a sensor data module 1210, and an advanced sleep data module 1214. The network interface 1200 includes hardware and low level software to allow hardware devices (e.g., components of the service 410a) to communicate over networks (e.g., with each other, with other destinations over the Internet 412). The network interface 1200 can include network cards, routers, modems, and other hardware. The communication manager 1202 generally includes hardware and software that operate above the network interface 1200 such as software to initiate, maintain, and tear down network communications used by the service 410a (e.g., TCP/IP, SSL or TLS, Torrent, and other communication sessions over local or wide area networks). The communication manager 1202 can also provide load balancing and other services to other elements of the service 410a. The server hardware 1204 generally includes physical processing devices used to instantiate and maintain the service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected. The server system software 1206 generally includes software that runs on the server hardware 1204 to provide operating environments to applications and services (e.g., operating systems running on real servers, virtual machines instantiated on real servers to create many virtual servers, server level operations such as data migration, redundancy, and backup).

The user identification 1208 can include, or reference, data related to users of beds with associated data processing systems. The users may include customers, owners, or other users registered with the service 410a or another service. Each user can have a unique identifier, user credentials, contact information, billing information, demographic information, or any other technologically appropriate information.

The device manager 1210 can include, or reference, data related to beds or other products associated with data processing systems. The beds can include products sold or registered with a system associated with the service 410a. Each bed can have a unique identifier, model and/or serial number, sales information, geographic information, delivery information, a listing of associated sensors and control peripherals, etc. An index or indexes stored by the service 410a can identify users associated with beds. This index can record sales of a bed to a user, users that sleep in a bed, etc.

The sensor data 1212 can record raw or condensed sensor data recorded by beds with associated data processing systems. For example, a bed's data processing system can have temperature, pressure, motion, audio, and/or light sensors. Readings from these sensors, either in raw form or in a format generated from the raw data (e.g. sleep metrics), can be communicated by the bed's data processing system to the service 410a for storage in the sensor data 1212. An index or indexes stored by the service 410a can identify users and/or beds associated with the sensor data 1212.

The service 410a can use any of its available data (e.g., sensor data 1212) to generate advanced sleep data 1214. The advanced sleep data 1214 includes sleep metrics and other data generated from sensor readings (e.g., health information). Some of these calculations can be performed in the service 410a instead of locally on the bed's data processing system because the calculations can be computationally complex or require a large amount of memory space or processor power that may not be available on the bed's data processing system. This can help allow a bed system to operate with a relatively simple controller while being part of a system that performs relatively complex tasks and computations.

For example, the service 410a can retrieve one or more machine learning models from a remote data store and use those models to determine the advanced sleep data 1214. The service 410a can retrieve one or more models to determine overall sleep quality of the user based on currently detected sensor data 1212 and/or historic sensor data. The service 410a can retrieve other models to determine whether the user is snoring based on the detected sensor data 1212. The service 410a can retrieve other models to determine whether the user experiences a health condition based on the data 1212.

FIG. 13 is a block diagram of an example sleep data cloud service 410b used in a data processing system associated with a bed system. Here, the sleep data cloud service 410b is configured to record data related to users' sleep experience. The service 410b includes a network interface 1300, a communication manager 1302, server hardware 1304, and server system software 1306. The service 410b also includes a user identification module 1308, a pressure sensor manager 1310, a pressure based sleep data module 1312, a raw pressure sensor data module 1314, and a non-pressure sleep data module 1316. Sometimes, the service 410b can include a sensor manager for each sensor. The service 410b can also include a sensor manager that relates to multiple sensors in beds (e.g., a single sensor manager can relate to pressure, temperature, light, movement, and audio sensors in a bed).

The pressure sensor manager 1310 can include, or reference, data related to the configuration and operation of pressure sensors in beds. This data can include an identifier of the types of sensors in a particular bed, their settings and calibration data, etc. The pressure based sleep data 1312 can use raw pressure sensor data 1314 to calculate sleep metrics tied to pressure sensor data. For example, user presence, movements, weight change, heartrate, and breathing rate can be determined from raw pressure sensor data 1314. An index or indexes stored by the service 410b can identify users associated with pressure sensors, raw pressure sensor data, and/or pressure based sleep data. The non-pressure sleep data 1316 can use other sources of data to calculate sleep metrics. User-entered preferences, light sensor readings, and sound sensor readings can be used to track sleep data. User presence can also be determined from a combination of raw pressure sensor data 1314 and non-pressure sleep data 1316 (e.g., raw temperature data). Sometimes, bed presence can be determined using only the temperature data. Changes in temperature data can be monitored to determine bed presence or absence in a temporal interval (e.g., window of time) of a given duration. The temperature and/or pressure data can also be combined with other sensing modalities or motion sensors that reflect different forms of movement (e.g., load cells) to accurately detect user presence. For example, the temperature and/or pressure data can be provided as input to a bed presence classifier, which can determine user bed presence based on real-time or near real-time data collected at the bed. The classifier can be trained to differentiate the temperature data from the pressure data, identify peak values in the temperature and pressure data, and generate a bed presence indication based on correlating the peak values. The peak values can be within a threshold distance from each other to then generate an indication that the user is in the bed. An index or indexes stored by the service 410b can identify users associated with sensors and/or the data 1316.

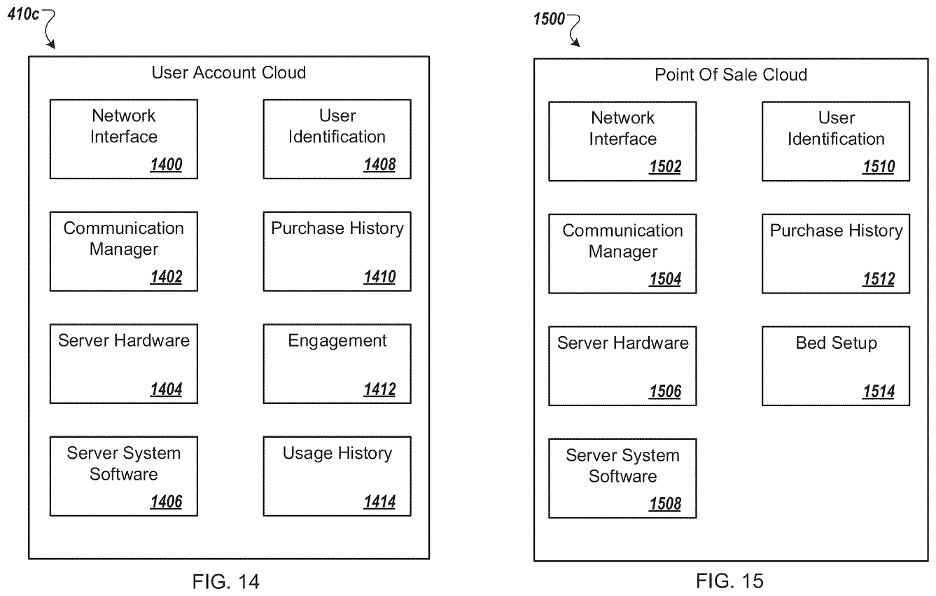

FIG. 14 is a block diagram of an example user account cloud service 410c used in a data processing system associated with a bed system. Here, the service 410c is configured to record a list of users and to identify other data related to those users. The service 410c includes a network interface 1400, a communication manager 1402, server hardware 1404, and server system software 1406. The service 410c also includes a user identification module 1408, a purchase history module 1410, an engagement module 1412, and an application usage history module 1414.

The user identification module 1408 can include, or reference, data related to users of beds with associated data processing systems, as described above. The purchase history module 1410 can include, or reference, data related to purchases by users. The purchase data can include a sale's contact information, billing information, and salesperson information associated with the user's purchase of the bed system. An index or indexes stored by the service 410c can identify users associated with a bed purchase.

The engagement module 1412 can track user interactions with the manufacturer, vendor, and/or manager of the bed/cloud services. This data can include communications (e.g., emails, service calls), data from sales (e.g., sales receipts, configuration logs), and social network interactions. The data can also include servicing, maintenance, or replacements of components of the user's bed system. The usage history module 1414 can contain data about user interactions with applications and/or remote controls of the bed. A monitoring and configuration application can be distributed to run on, for example, computing devices 412 described herein. The application can log and report user interactions for storage in the application usage history module 1414. An index or indexes stored by the service 410c can also identify users associated with each log entry. User interactions stored in the module 1414 can optionally be used to determine or predict user preferences and/or settings for the user's bed and/or peripheral devices that can improve the user's overall sleep quality.

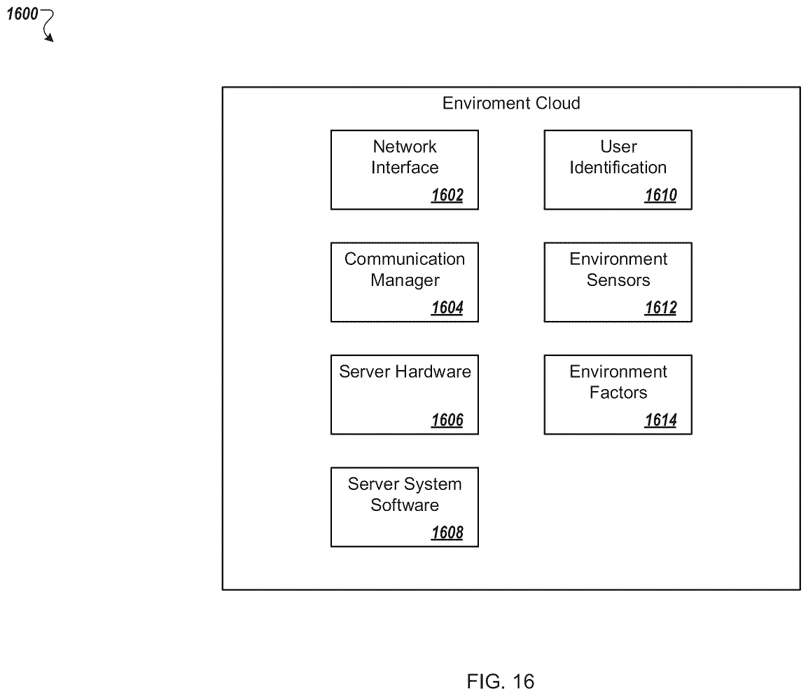

FIG. 15 is a block diagram of an example point of sale cloud service 1500 used in a data processing system associated with a bed system. Here, the service 1500 can record data related to users' purchases, specifically purchases of bed systems described herein. The service 1500 is shown with a network interface 1502, a communication manager 1504, server hardware 1506, and server system software 1508. The service 1500 also includes a user identification module 1510, a purchase history module 1512, and a bed setup module 1514.

The purchase history module 1512 can include, or reference, data related to purchases made by users identified in the module 1510, such as data of a sale, price, and location of sale, delivery address, and configuration options selected by the users at the time of sale. The configuration options can include selections made by the user about how they wish their newly purchased beds to be setup and can include expected sleep schedule, a listing of peripheral sensors and controllers that they have or will install, etc.

The bed setup module 1514 can include, or reference, data related to installations of beds that users purchase. The bed setup data can include a date and address to which a bed is delivered, a person who accepts delivery, configuration that is applied to the bed upon delivery (e.g., firmness settings), name(s) of bed user(s), which side of the bed each user will use, etc. Data recorded in the service 1500 can be referenced by a user's bed system at later times to control functionality of the bed system and/or to send control signals to peripheral components. This can allow a salesperson to collect information from the user at the point of sale that later facilitates bed system automation. Sometimes, some or all aspects of the bed system can be automated with little or no user-entered data required after the point of sale. Sometimes, data recorded in the service 1500 can be used in connection with other, user-entered data.

FIG. 16 is a block diagram of an example environment cloud service 1600 used in a data processing system associated with a bed system. Here, the service 1600 is configured to record data related to users' home environment. The service 1600 includes a network interface 1602, a communication manager 1604, server hardware 1606, and server system software 1608. The service 1600 also includes a user identification module 1610, an environmental sensors module 1612, and an environmental factors module 1614. The environmental sensors module 1612 can include a listing and identification of sensors that users identified in the module 1610 to have installed in and/or surrounding their bed (e.g., light, noise/audio, vibration, thermostats, movement/motion sensors). The module 1612 can also store historical readings or reports from the environmental sensors. The module 1612 can be accessed at a later time and used by one or more cloud services described herein to determine sleep quality and/or health information of the users. The environmental factors module 1614 can include reports generated based on data in the module 1612. For example, the module 1614 can generate and retain a report indicating frequency and duration of instances of increased lighting when the user is asleep based on light sensor data that is stored in the environment sensors module 1612.

In the examples discussed here, each cloud service 410 is shown with some of the same components. These same components can be partially or wholly shared between services, or they can be separate. Sometimes, each service can have separate copies of some or all the components that are the same or different in some ways. These components are provided as illustrative examples. In other examples, each cloud service can have different number, types, and styles of components that are technically possible.

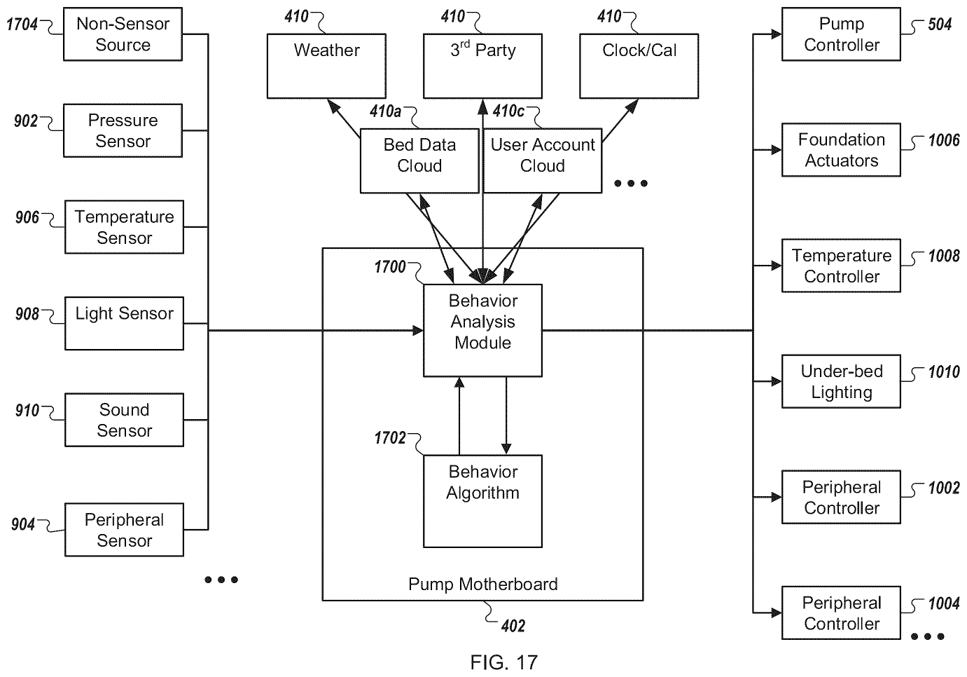

FIG. 17 is a block diagram of an example of using a data processing system associated with a bed to automate peripherals around the bed. Shown here is a behavior analysis module 1700 that runs on the motherboard 402. The behavior analysis module 1700 can be one or more software components stored on the computer memory 512 and executed by the processor 502. In general, the module 1700 can collect data from a variety of sources (e.g., sensors 902, 904, 906, 908, and/or 910, non-sensor local sources 1704, cloud data services 410a and/or 410c) and use a behavioral algorithm 1702 (e.g., machine learning model(s)) to generate actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The module 1700 can collect data from any technologically appropriate source (e.g., sensors of the sensor array 406) to gather data about features of a bed, the bed's environment, and/or the bed's users. The data can provide the module 1700 with information about a current state of the bed's environment. For example, the module 1700 can access readings from the pressure sensor 902 to determine air chamber pressure in the bed. From this reading, and potentially other data, user presence can be determined. In another example, the module 1700 can access the light sensor 908 to detect the amount of light in the environment. The module 1700 can also access the temperature sensor 906 to detect a temperature in the environment and/or microclimates in the bed. Using this data, the module 1700 can determine whether temperature adjustments should be made to the environment and/or components of the bed to improve the user's sleep quality and overall comfortability. Similarly, the module 1700 can access data from cloud services to make more accurate determinations of user sleep quality, health information, and/or control the bed and/or peripheral devices. For example, the behavior analysis module 1700 can access the bed cloud service 410a to access historical sensor data 1212 and/or advanced sleep data 1214. The module 1700 can also access a weather reporting service, a 3^{rd} party data provider (e.g., traffic and news data, emergency broadcast data, user travel data), and/or a clock and calendar service. Using data retrieved from the cloud services 410, the module 1700 can accurately determine user sleep quality, health information, and/or control of the bed and/or peripheral devices. Similarly, the module 1700 can access data from non-sensor sources 1704, such as a local clock and calendar service (e.g., a component of the motherboard 402 or of the processor 502). The module 1700 can use this information to determine, for example, times of day that the user is in bed, asleep, waking up, and/or going to bed.

The behavior analysis module 1700 can aggregate and prepare this data for use with one or more behavioral algorithms 1702 (e.g., machine learning models). The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

Here, the module 1700 and the behavioral algorithm 1702 are shown as components of the motherboard 402. Other configurations are also possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

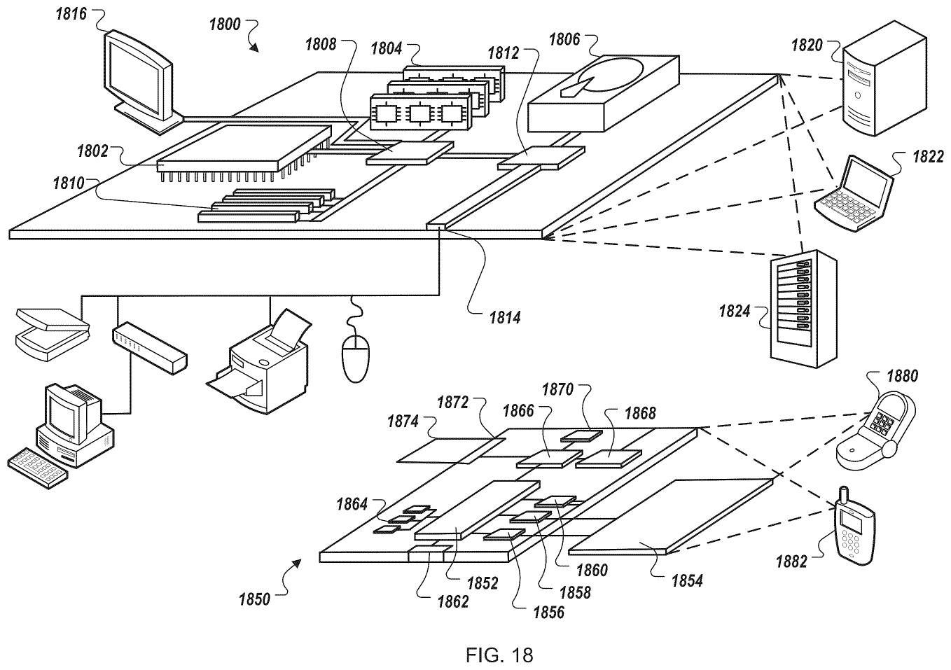

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system). The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk. The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter. The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other. The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850. The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850. The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850. The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input. The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet. The computing system can include clients and servers. A client and server are generally remote from each other and typically interact through a communication network. The relationship of client and server arises by virtue of computer programs running on the respective computers and having a client-server relationship to each other.

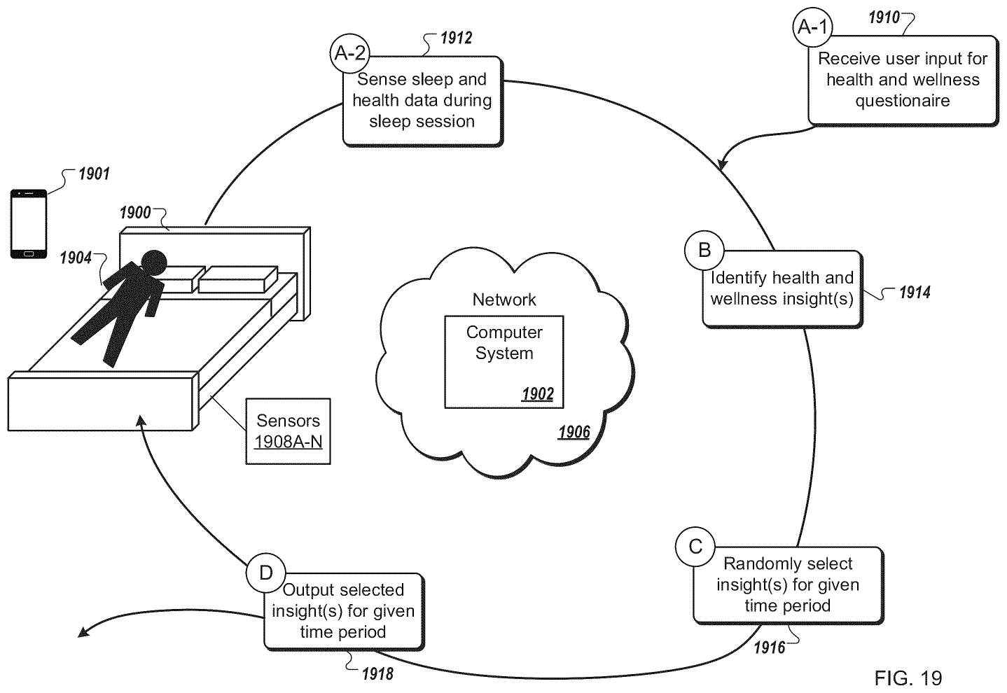

FIG. 19 is a conceptual diagram for determining personalized insights for a user 1904 of a bed system 1900. As depicted, the user 1904 can be sleeping in a bed system 1900. The bed system 1900 and/or a user device 1901 (e.g., mobile device, mobile phone, smartphone, wearable device, laptop, tablet, computer, automation hub, home automation device) can be in data communication (e.g., wired, wireless) with a computer system 1902 via network 1906.

The computer system 1902 can be configured to perform a variety of smart-bed related tasks including generating personalized insights (e.g., sleep recommendations) for the user 1904, determining a sleep state of the user 1904, and making adjustments to the bed system 1900 or a surrounding environment. The computer system 1902 can be separate/remote from the bed system 1900 (e.g., cloud-based computing system). In some implementations, the computer system 1902 can be part of the bed system 1900, such as a controller or processor of the bed system 1900.

The user device 1901 can be configured to present information to the user 1904 about their sleep environment, their sleep health/quality, overall health, smart-bed related tasks, and/or insights. The user device 1901 can be the same as or similar to any of the mobile devices, computing devices, and/or user devices described throughout this disclosure.

As described herein, the bed system 1900 can include a plurality of sensors 1908A-N (e.g., a sensor system) configured to detect pressure, temperature, and other indicators of the user 1904 when the user 1904 lays on top of a mattress of the bed system 1900. In some implementations, the sensors 1908A-N can be part of wearable devices (e.g., smart watch, heart rate monitor, smart clothes, etc.) and/or mobile phones or home automation devices that are in data communication with the computer system 1902. Any one or more of the sensors described herein can capture presence, movement, and biometric information about the user 1904, for example. The plurality of sensors 1908A-N can include any combination of sensors described throughout this disclosure (e.g., refer to FIGS. 1-18).

The sensed information can include a variety of different signals. For example, the information can include audio waves indicative of breathing and/or snoring of the user 1904. The information can include pressure in the mattress indicative of movement of the user 1904 on top of the mattress. The information can also include pressure changes in one or more air chambers or sections of the mattress indicative of the user 1904 being on top of the mattress. The information can also include pressure changes or other measurements indicative of the user 1904's heartbeat, breathing rate, and/or respiration rate. Moreover, the information can include temperature of the user 1904. The information can also indicate changes in temperature at a top surface of the mattress indicative that the user 1904 is on top of the mattress. The information can include any one or more additional measurements that can be used to determine that the user 1904 is presently on top of the mattress/in the bed system 1900.

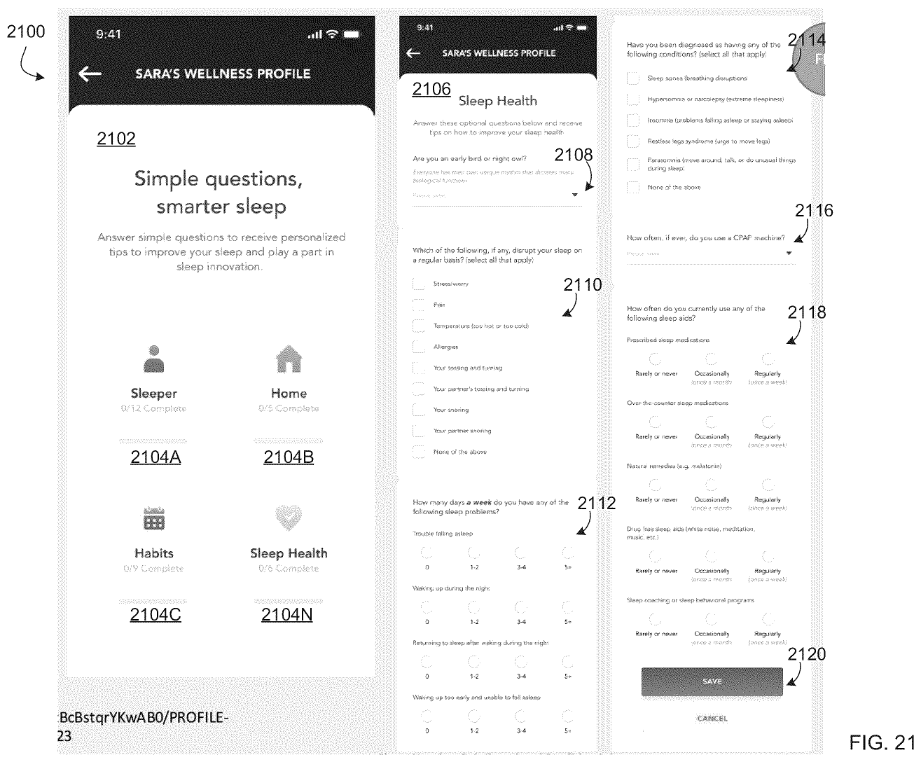

The user 1904 can view a health and wellness questionnaire at the user device 1901 any time before or after they go to sleep in the bed system 1900. An example questionnaire is depicted and described further in reference to FIG. 21. The questionnaire can present the user 1904 with questions about their sleep habits and subjective perceptions of their sleep health and/or quality. The user 1904 can answer questions in the questionnaire, thereby providing user input to the computer system 1902 (block A-1, 1910). The user 1904 can answer the questions once. The user 1904 may also go back and update or change their answers to any of the questions at one or more other times.

Using the user 1904's responses to the questionnaire (which can be transmitted by the user device 101 to the computer system 1902 over the network(s) 1906), the computer system 1902 can identify one or more health and wellness insights (e.g., personalized insights) relevant to the particular user 1904 (block B, 1914). As described further below, one or more of the insights may also be determined without using the user 1904's responses to the questionnaire. For example, the insights can be determined based on one or more of sensor data, rules, algorithms, and/or machine learning models. Tracking specific health and/or wellness conditions, such as circadian chronotype, may also be estimated and/or determined using sensor-enabled rules, algorithms, and/or machine learning techniques. The health and wellness insights can be generated by the computer system 1902 based on historic sleep and health data about the user 1904 and/or a general population of users of similar bed systems 1900. The computer system 1902 can leverage this abundance of historic data in order to curate general and personalized insights intended to improve users' overall sleep health, sleep quality, and/or health. The computer system 1902 can store (e.g., in a data repository) these insights and update them over time, such as based on outcomes from other users taking action on the insights that are selected, by the computer system 1902, and presented to them at their respective devices. Thus, the computer system 1902 can leverage existing data and analytics of sleep behavior and/or quality of many users to efficiently and accurately select health and wellness insights intended to improve the particular user 1904's overall sleep quality and/or health.

For example, the computer system 1902 can determine a circadian chronotype of the user 1904 based on their answers to the health and wellness questionnaire. The circadian chronotype can correspond to their circadian rhythm. For example, circadian chronotypes can correspond to circadian rhythms of night owls, early birds, and neutral (e.g., neither a night owl nor an early bird). The circadian chronotypes may also be a first type, a second type, and/or a third type. Sometimes, the circadian chronotypes can include more than three types. The circadian chronotypes can also include less than three types. Any of the types of circadian chronotypes can be defined by the computer system 1902 or relevant stakeholders that implement the disclosed technology.

The computer system 1902 can determine the circadian chronotype of the user 1904 using a ruleset that maps answers to the health and wellness questionnaire with indicators of different circadian rhythms. The computer system 1902 can therefore correlate the user 1904's answers to the questionnaire with potential circadian rhythms to determine a circadian rhythm of the user 1904, and therefore the user 1904's circadian chronotype. Based on the determined circadian chronotype of the user 1904, the computer system 1902 can then select a set of health and/or wellness insights, as described above, that can be directed to maintaining and/or improving the particular circadian chronotype of the user 1904.

In some implementations, the computer system 1902 may also leverage real-time sensor data collected by the sensors 1908A-N of the bed system 1900 (and/or the user device 1901) to generate and/or select the health and/or wellness insights in block B (1914). The computer system 1902 can also leverage the sensor data to determine a circadian chronotype of the user 1904, as described further below. The sensors 1908A-N can be used to sense health and/or health data about a sleep session of the user 1904 (block A-2, 1912). The sensors 1908A-N may include pressure sensors, as described herein, which can detect respiration rate, HR, and/or user presence in the bed system 1900. The sensors 1908A-N may also include temperature or thermal sensors, load cells, or other types of sensing devices that may detect a variety of data about the user 1904, all of which may be processed and used by the computer system 1902 to accurately curate a set of insights that may help maintain the user 1904's circadian rhythm and/or improve their overall sleep quality and/or health.

The sensed data can be transmitted to the computer system 1902 (e.g., over the network 1906). The data can be transmitted as it is sensed. In some implementations, the bed system 1900 can be configured to sense information at predetermined time intervals. At an end of each of the time intervals, the bed system 1900 can transmit the sensed data to the computer system 1902. Moreover, in some implementations, the bed system 1900 can receive a request from the computer system 1902 to sense information of the user 1904. At that point, the bed system 1900 can sense the information and transmit the sensed data to the computer system 1902. The computer system 1902 can then identify the health and/or wellness insights in block B (1914) based on a combination of the user input to the questionnaire and the sensed data.

Sometimes, the computer system 1902 can leverage sensed data collected over time, such as heartrate (HR) or heartrate variability (HRV) to measure stress levels of the user 1904. HR and/or HRV data can be used to select one or more insights that may help the user 1904 build habits that can reduce stress over time, maintain the user 1904's circadian rhythm, and/or improve the user 1904's overall sleep quality and/or health. Similarly, the computer system 1902 can leverage other sensed data collected over time (e.g., over multiple sleep sessions) for the particular user 1904 to determine and select other insights to be presented to the user 1904 that can help the user 1904 get back onto routine that maintains their circadian rhythm. Other historic data about the user 1904's sleep patterns, sleep behavior, and/or health information can also be leveraged by the computer system 1902 to determine a circadian chronotype of the user 1904 and/or insights, tips, and/or recommendations that may be provided to the user 1904 to improve the user 1904's overall sleep health and/or quality.

The computer system 1902 may store a ruleset that defines a series of parameters such as sleep quality (e.g., on a scale of 1-100), subjective sleep quality (e.g., wakefulness on a 1-10 scale), time (e.g. hours, minutes, and seconds of the twenty-four hour day, time since the user has awoken, time since the user has exited their bed), user behavior activity (e.g. typical exercise and diet habits and calendar appointments). The computer system 1902 may apply the received data for the user and their sleep session to this ruleset and may produce or select one or more behavioral recommendations (e.g., health and/or wellness insights) for the user 1904. These recommendations may be presented to the user on their user device 1901.

In block C (1916), the computer system 1902 can randomly select at least one insight from the set of identified health and wellness insights for a given time period. The computer system 1902 can utilize randomized logic to determine which insight to select and present to the user for the given time period. The given time period can include any amount of predetermined time. For example, the computer system 1902 can determine which insight to present to the user 1904 when the user 1904 wakes up from a current sleep session (e.g., once the user unlocks their user device 1901 and opens an application where the questionnaire was presented). The computer system 1902 can determine which insight to present to the user 1904 as a notification during the day or between sleep sessions. The computer system 1902 may determine which insight to present in which GUI screen at the user 1904's device 1901. Moreover, the computer system 1902 can determine which insight or insights to elevate/prioritize over others for presentation during the given time period. In some implementations, as described herein, the computer system 1902 can leverage intelligence machine learning rules and/or logic to determine which insights may be most relevant to the user and therefore should be presented during the given time period. In some implementations, the computer system 1902 can leverage a combination of randomized logic and machine learning rules logic to determine which insights to present during the given time period.

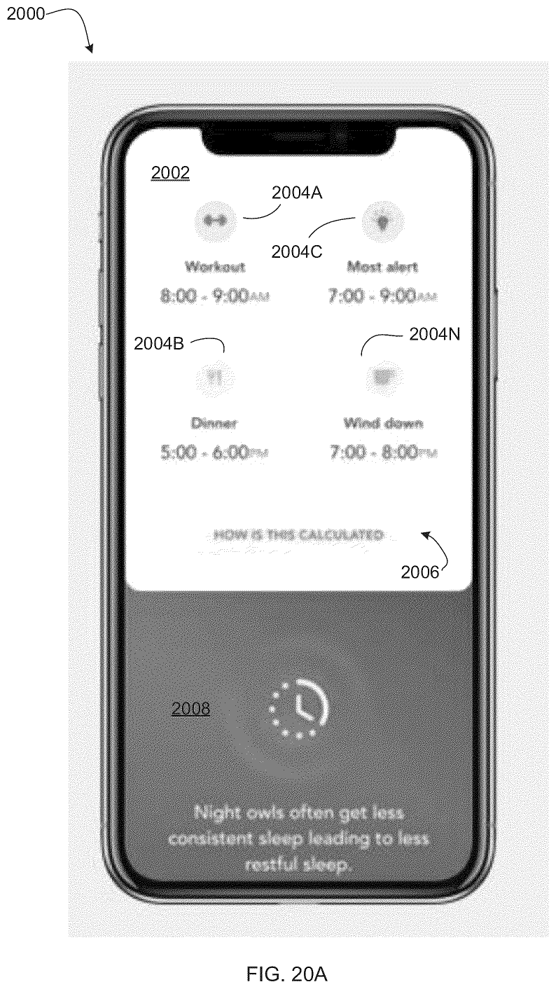

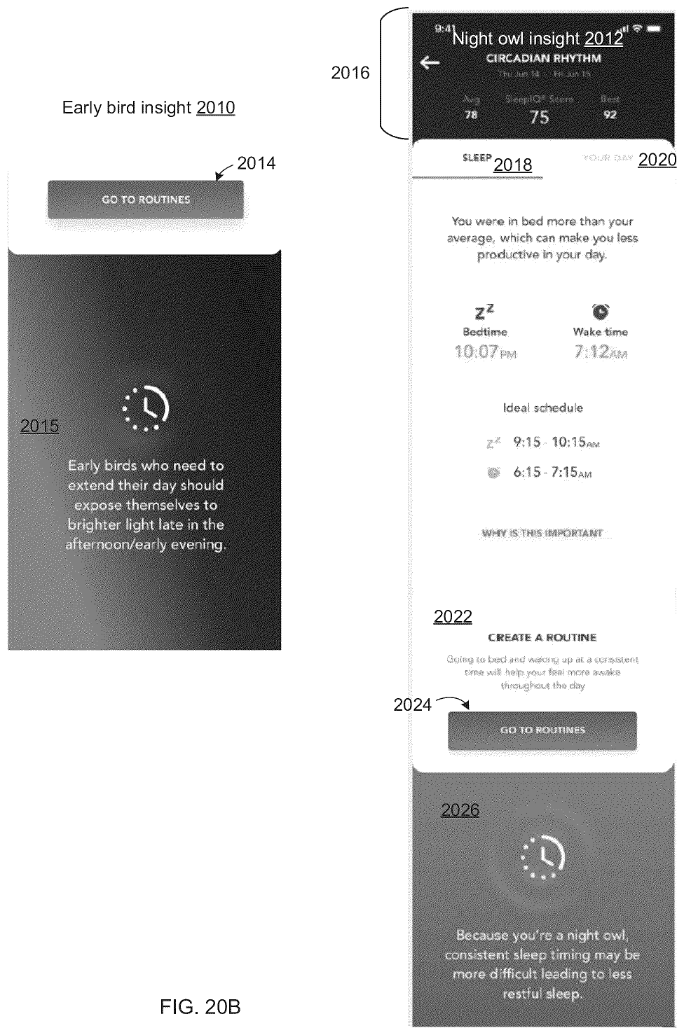

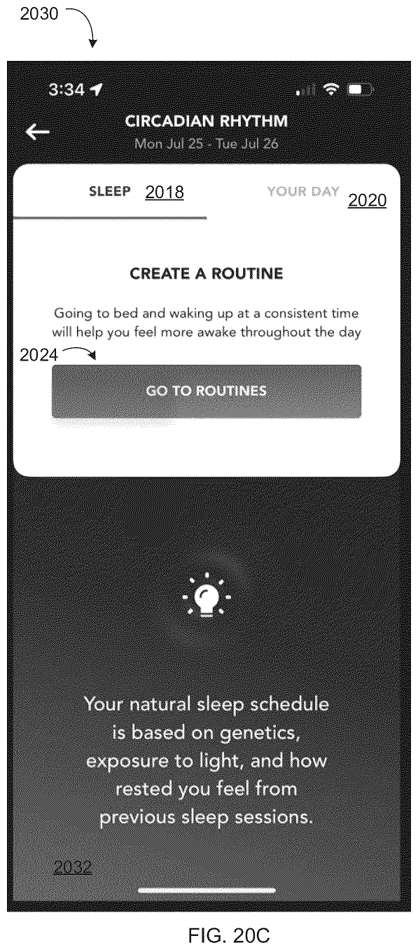

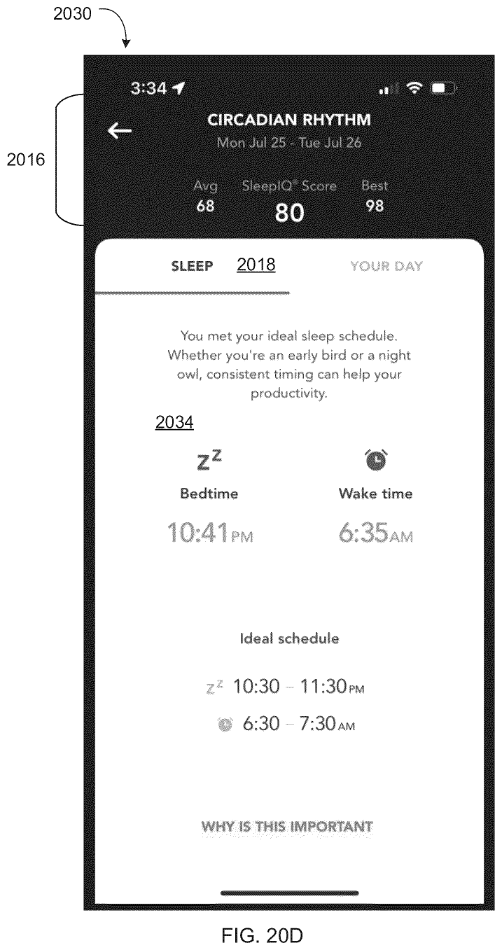

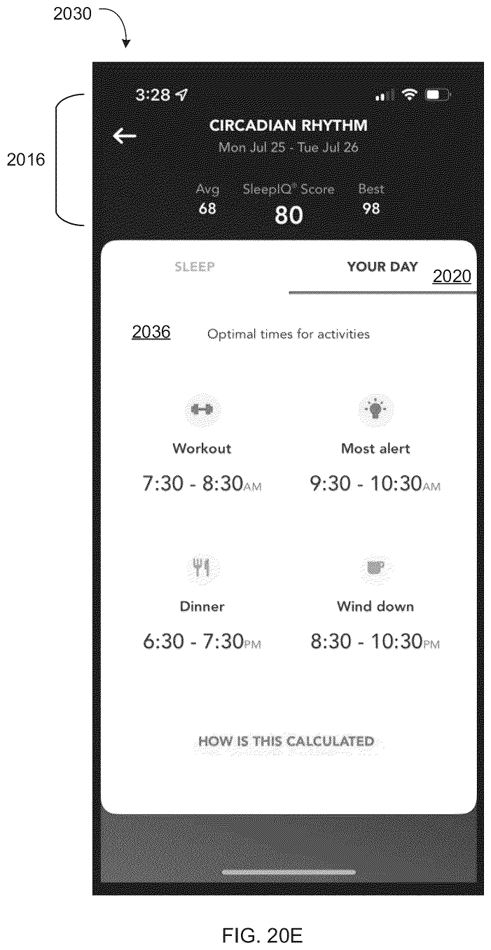

The computer system 1902 can then output the selected insight(s) for the given time period (block D, 1918). As described herein, the insight(s) can be presented in a GUI screen at the user 1904's device 1901. The insight(s) can be presented as a notification at the user 1904's device 1901. The insight(s) can be presented as a pop-out window in one or more GUI screens at the user 1904's device 1901, as the user 1904 navigates to different GUI screens in a mobile application at the device. Refer to FIGS. 20A-B for further discussion about presenting the insight(s) to the user 1904. In some implementations, the selected insight(s) can be presented at one or more other wearable devices, home automation hubs, or other user/computing devices that are in communication with the computer system 1902.

FIGS. 20A-B are example graphical user interfaces (GUIs) for presenting personalized insights to a user. As described above, the GUIs depicted and described herein can be presented at a mobile device of the user (e.g., sleeper) of a bed system. The mobile device can be a smartphone, phone, laptop, tablet, wearable device, or any other device of the user.

In FIG. 20A, GUI 2000 presents suggested times at which a particular user should perform one or more actions during a current time period. The current time period can be a present day. In some implementations, the current time period can include multiple days, such as a current day and a next day, a next day, a next three days, a next five days, a next seven days, etc. The suggested times can be determined, by a computing system, based on a circadian chronotype of the user. The circadian chronotype, as described herein, can be determined based on subjective responses that the user provides to a health and wellness questionnaire, such as the questionnaire depicted and described in FIG. 21. Moreover, each day (or each next predetermined time period) the suggested times can be adjusted based on new or updated responses that the user provides to the questionnaire. The suggested times can also be adjusted based on data sensed at the bed system when the user is in bed and/or during sleep sessions.

The GUI 2000 includes a panel 2002 and a tip 2008. The user can scroll up and down on a screen of their mobile device to view more information in the panel 2002 or less information therein. For example, the user can make a scrolling motion from a bottom of the mobile device screen to a top of the screen in order to view more of the tip 2008 in the screen and less or none of the panel 2002. The user may also continue scrolling to view additional information associated with the personalized insight(s) being presented at the mobile device. The user can access and view the panel 2002 and their personalized insights from various GUI screens presented in a mobile application at the user's mobile device. As an illustrative example, the panel 2002 can be presented in a GUI screen that presents information about the user's circadian rhythm. As another example, the panel 2002 can be presented in a home screen or greeting screen of the mobile application when the application is launched at the user's mobile device (e.g., in some scenarios when the personalized insights may be applicable to the user first entering the mobile application for a day or after a sleep session). In some implementations, the panel 2002 can be displayed in a GUI screen at the user's mobile device whenever the personalized insights therein may be most relevant to the user's experience with the mobile application. For example, machine learning rules and logic can be used to weight one or more of the personalized insights, present most relevant insights to the user each day, and/or determine the most relevant insights to the user's experience with the mobile application. In some implementations, any of the personalized insights may also be provided to the user as notifications at the mobile device.

The panel 2002 includes suggested times that the user should perform actions 2004A-N. The panel 2002 can include one or more additional or fewer actions and corresponding suggested times. Here, the action 2004A is a workout or physical activity routine. The computer system described herein can determine, based on the user's responses to the health and wellness questionnaire, that the user can maintain their circadian rhythm and improve their overall sleep quality and/or health by working out in the morning from 8:00-9:00 AM. This suggested time is presented in the panel 2002 with a graphical icon corresponding to the action 2004A of working out (e.g., a weight).

The action 2004B is a meal routine, such as eating dinner. Based on the user's questionnaire responses, the computer system can determine that the user can maintain their circadian rhythm and improve their sleep quality and/or health if they eat dinner between 5:00-6:00 PM. This suggested time is presented in the panel 2002 with a graphical icon corresponding to the action 2004B of eating a meal (e.g., a fork and knife).

The action 2004C is an alertness level. The computer system described herein can determine, based on the user's responses to the health and wellness questionnaire, that the user may be most alert from 7:00-9:00 AM. This suggested time is presented in the panel 2002 with a graphical icon corresponding to the action 2004C of most alertness (e.g., a lightbulb).

The action 2004N is a winding down routine. The winding down routine can include any activities the user may perform to prepare for going to bed or another sleep session, such as relaxing, reading a book, having a cup of tea, etc. The computer system described herein can determine, based on the user's responses to the questionnaire, that the user can maintain their circadian rhythm and improve their overall sleep quality and/or health by starting to wind down for a sleep session between 7:00-8:00 PM. This suggested time is presented in the panel 2002 with a graphical icon corresponding to the action 2004N of winding down (e.g., a cup of tea).

Any of the actions 2004A-N can be performed during the current time period. The user can also perform less than all of the actions 2004A-N at the suggested times presented in the GUI 2000. As described herein, the suggested times for the actions 2004A-N can be presented in such a way that gently nudges the user to build habits that maintain their circadian rhythm and/or improve their sleep quality and/or health. By gently nudging the user to build these habits, the user can develop a sense of agency that they are making a change in their overall health.

The panel 2002 can also include a selectable option (e.g., button) 2006. The user can select the option 2006 to learn how the suggested times for the actions 2004A-N are calculated (e.g., determined, selected, identified). Upon selecting the option 2006, additional information can be presented in the GUI 2000, such as in a pop-out window and/or in the panel 2002. In some implementations, selecting the option 2006 can cause another GUI to replace the GUI 2000 and be presented at the mobile device. In response to the user selecting the option 2006, the computer system can pull relevant information to present from a data repository. Therefore, the user can be presented with information that makes them more informed about their habits, how to improve them, and how to improve their overall sleep quality and/or health.

The tip 2008 can indicate at least one informative piece of information that educates the user about their circadian rhythm and/or their particular circadian chronotype. The tip 2008 can be randomly selected, by the computer system, to be presented to the user during the current time period. Once the current time period expires (e.g., a new day begins, the user closes out of the application at the mobile device and opens the application at another, later time), the computer system can use randomized logic to select another tip to be presented in the GUI 2000. In the example of FIG. 20A, the tip 2008 indicates that night owls often get less consistent sleep than early birds, which leads to less restful sleep (and therefore lower quality sleep). The tip 2008 can be part of a set of insights that correspond to a circadian chronotype of a night owl. Similarly, the suggested times for the actions 2004A-N can correspond to the circadian chronotype of the night owl. Here, once the user responded to the sleep and health questionnaire, the computer system determined that the user has the circadian chronotype of the night owl, and thus generated the suggested times for the actions 2004A-N and selected the tip 2008 to present in the GUI 2000.

Although multiple users may have the same circadian chronotype of the night owl, not every user may receive the same suggested times for the actions 2004A-N and/or the tip 2008. The suggested times for the actions 2004A-N can be determined, by the computer system, based on the particular responses of a user to the questionnaire. Optionally, the suggested times for the actions 2004A-N can also be determined, by the computer system, based on historic sleeper data associated with the particular user and/or sensor data sensed by sensors of the bed system during sleep sessions of the particular user. Accordingly, the information presented in the GUI 2000 can be personalized for each user of a bed system. Moreover, each user can receive a different tip since the computer system utilizes randomized logic to select and present tips to the users.

FIG. 20B illustrates example GUIs 2010 and 2012 for different circadian chronotypes. The GUI 2010 presents one or more tips that may be presented to a user having a circadian chronotype of an early bird. The example GUI 2010 includes a selectable option 2014 to go to the user's routines. Selecting the option 2014 can cause a new GUI (or a pop-out window or other graphical element) to be presented at the user's mobile device. The new GUI can present one or more recommended actions and/or suggested times for performing actions that maintain the user's circadian rhythm and/or are intended to improve the user's overall sleep quality and/or health. The recommended actions can include, for example, when to workout, when the user is most alert, when to eat dinner, and/or when to wind down. Refer to the GUI 2000 in FIG. 20A for additional discussion about the recommended actions.

In some implementations, selecting the option 2014 can cause another GUI to be presented at the user's mobile device indicating one or more routines that the user has created and/or one or more routines that the computer system automatically generated/suggested for the user (e.g., based on the user's responses to the questionnaire, historic sleeper/health data about the user, sensed data about the user during one or more sleep sessions). For example, refer to example GUI 2012 in FIG. 20B for additional discussion. The routines can include times at which the user desires to go to bed and/or wakeup. The routines can include suggested times at which the user should go to bed and/or wakeup. The routines can also include times at which the user actually goes to bed and/or wakes up. The routines may also include one or more other activities that the user desires to perform, should perform, and/or actually performs throughout their day. The routines can be intended to provide the user with both in-application and notifications to guide the user to optimize their overall sleep health. Any of the routines may also consist of both individual insights as well as recommendations about how to customize the user's smart bed to optimize their sleep health.

The GUI 2010 for the circadian chronotype of the early bird may also include a tip 2015. As described herein, the tip 2015 can be randomly selected from a set of tips that correspond to the circadian chronotype of the early bird. As described throughout this disclosure, machine learning rules may also be used to determine a most relevant tip to present to the user. In the example of the GUI 2010, the tip 2015 indicates that early birds who need to extent their day should expose themselves to brighter light late in the afternoon and/or early evening. The tip 2015 can help educate the particular sleeper about one or more actions they can take in order to maintain their circadian rhythm and/or improve their sleep quality and/or health.

In some implementations, the user can use the tip 2015 to adjust home automation devices such that the home automation devices align with the suggested actions that may maintain the user's circadian rhythm and/or improve their sleep quality and/or health. As an illustrative example, with the tip 2015, the user may decide to set a routine in which lights in the user's bedroom remain bright and on during the early evening. To set the routine, the user may select the option 2014 and follow prompts presented in the next GUI. One or more other actions can be taken by the user in order to act on the tip 2015 that is presented in the GUI 2010.

Although the GUI 2010 is depicted and described in terms of a circadian chronotype of an early bird, the same and/or similar features (e.g., selectable option 2014 and tip 2015) can be presented in a GUI for a circadian chronotype of a night owl with insights that pertain to night owls.

Still referring to FIG. 20B, GUI 2012 presents one or more tips and/or routines for a circadian chronotype of a night owl. The GUI 2012 can present several features 2016, 2018, 2020, 2022, and/or 2026 that may not be presented in a GUI display at the mobile device all at once. In other words, the user can scroll (e.g., with a scrolling motion from a bottom to a top of the screen of the mobile device) through the features presented at the mobile device to view one or more of the features. For example, the feature 2016 and at least a portion of the feature 2018 can be presented at the user's mobile device. The user can then scroll to view at least a portion of the feature 2022 and/or a portion of the feature 2026. When the user views at least a portion of the feature 2022, at least a portion of the feature 2016 and/or the feature 2018 may no longer be visible on the GUI display/screen of the mobile device. However, the user can scroll back up to view either of the features 2016 and/or 2018. How much and/or how many of the features that are presented at the mobile device at one time may also vary depending on screen ratio information associated with the user's particular mobile device.

In the GUI 2012, the feature 2016 can be a panel providing a high level overview of information associated with the user's sleep quality and/or health. For example, the feature 2016 can include information about a particular aspect of the user's sleep quality and/or health that is presented in the GUI 2012. Here, the particular aspect of the user's sleep quality and/or health is circadian rhythm. The information presented in the GUI 2012 can also correspond to a particular period of time, such as a current/present day, a time interval between consecutive sleep sessions (e.g., time between two sleep sessions, time between three sleep sessions, time between 5 sleep sessions). The feature 2016 can also include information such as the user's average sleep score, a sleep score for the user's most recent/last sleep session (e.g., a previous night), and a best sleep score of the user. Any of these scores can be determined by the computer system and using the techniques described herein (e.g., by receiving and processing sensor data from a variety of sensors of the bed system).

The feature 2018 can be a panel having information about the most recent/last sleep session of the user. The feature 2018 can include a tip about how the user's most recent sleep activity may affect their overall circadian rhythm and/or sleep quality and/or health. As an illustrative example, the feature 2018 in FIG. 20B presents an indication that the user was in bed for a longer amount of time than their average and as a result, the user may be less productive during the upcoming day (e.g., time between a current time and a next sleep session). The computer system can determine what information to present as the tip in the feature 2018 based on analyzing sensor data from the sensors of the bed system and classifying the sensor data with known information associated with the circadian chronotype of a night owl. For example, the computer system can retrieve historic data about the user and/or a general population of users from a data repository, the data corresponding to night owls. The computer system can use rulesets to compare the sensor data (e.g., a time the user fell asleep, how long the user was asleep, a time the user woke up) to known historic data about other night owls to determine whether the user's sleep routine maintains the user's circadian rhythm and/or negatively impacts their sleep health and/or quality. Based on the computer system's comparison of the sensor data to the historic data, the computer system can accurately select and/or generate an insight, such as the tip presented in the feature 2018, to present to the user and inform them about their current sleep habits.

The feature 2018 may also include information such as a time that the user went to bed (e.g., got into bed, fell asleep) and a time that the user woke up during the most recent/last sleep session. This timing information can be determined based on sensor data received from sensors of the bed system during the user's sleep session. The feature 2018 can also include suggested times at which the user should go to sleep and/or wake up. As described herein, the computer system can determine the suggested times based on the user's circadian chronotype as a night owl. This determination can also be made based on analyzing, by the computer system, sensed data during the user's most recent sleep session and/or historic sleep sessions, historic data about the user or a general population of similar users, and/or the user's responses to the health and wellness questionnaire described herein. Moreover, the feature 2018 can include a selectable option to learn more about why it may be important for the user to adjust their sleeping habits and try to follow the ideal schedule suggested by the computer system and presented in the GUI 2012.

The feature 2020 can be a selectable tab in the GUI 2012. By selecting the feature 2020, the information presented in the feature 2018 can be replaced with information corresponding to the feature 2020. In the example of FIG. 20B, the feature 2020 can include information about the user's upcoming day (or another predetermined period of time, such as an amount of time between consecutive sleep sessions). The information about the user's upcoming day can include a recommended daytime schedule. The recommended daytime schedule may include, but is not limited to, recommendations for when the user should eat meals, when the user should work out, when the user should wind down, and/or when the user is alert. The information may also include guidance and/or instructions about how the user can set up or update one or more of their daytime routines.

The feature 2022 can be a panel having information about the user's routine(s). For example, the feature 2022 can include instructions about how to create a routine. The feature 2022 may also include one or more tips that gently nudge the user to create routines that maintain/improve their circadian rhythm and/or improve their overall sleep quality and/or health. In the example of FIG. 20B, the feature 2022 includes a tip that going to bed and waking up at a consistent time can help the user feel more awake during the day. As described herein, the computer system can use randomized logic (and/or machine learning rules) to select what tip to present in the feature 2022 when the user views the GUI 2012 at their mobile device.

The feature 2022 can also include a selectable option 2024 to go to their routines. Selecting the option 2024 can cause the GUI 2012 to be replaced with a new GUI that displays information about the user's current routines. The new GUI may also present selectable options for the user to create new routines and/or update/modify/remove current routines. The new GUI may also present one or more tips to help gently nudge the user to make routines that maintain their circadian rhythm and/or improve their overall sleep quality and/or health.

The feature 2026 can be a tip that is randomly selected by the computer system and based on the user's circadian chronotype of being a night owl. The feature 2026 can correspond to one or more other tips/suggestions presented in the features 2018 and/or 2022 to help gently nudge the user to modify their sleep habits to maintain their circadian rhythm and/or improve their overall sleep quality and/or health. In the example of FIG. 20B, the feature 2026 includes a tip indicating that since the user is a night owl, consistent sleep timing can be more difficult to achieve, which can cause the user to experience less restful sleep. As described herein, one or more other tips can be randomly selected and presented in any one or more of the features 2018, 2022, and/or 2026 to gently nudge the user to modify their sleep habits.

Although the GUI 2012 is depicted and described in terms of a circadian chronotype of a night owl, the same and/or similar features 2016, 2018, 2020, 2022, 2024, and/or 2026 can be presented in a GUI for a circadian chronotype of an early bird with insights and other information that pertain to early birds.

FIG. 20C illustrates an example GUI 2030 similar to the GUI 2012 discussed in reference to FIG. 20B. The GUI 2030 can be circadian rhythm screen presented in a mobile application at the user's mobile device. The GUI 2030 can present tips, insights, recommendations, and/or routines for a user based on their determined circadian chronotype. Like the GUI 2012, the GUI 2030 includes selectable features (e.g., tabs) 2018 and 2020. The user can toggle between the features 2018 and 2020 by clicking on them to view information corresponding to them.

Here, the feature 2018 is selected, which means the GUI 2030 is presenting sleep information as it relates to the user's circadian chronotype. The user can view routines that the user created and/or have been created for the user based on their circadian chronotype. To view the routines, the user can select the option 2024, described above. Refer to FIG. 20D for further discussion about viewing the routines. The GUI 2030 also presents an insight or tip 2032, which can be randomly selected or selected using machine learning rules, as described throughout this disclosure.

FIG. 20D illustrates the GUI 2030 that has been updated once the user selects the option 2024 to view their routines in FIG. 20C. As shown here, the feature 2016 is outputted to show a high level overview of the user's average sleep quality score, a current sleep quality score, and/or their best sleep quality score. One or more other sleep-related parameters may also be displayed in the feature 2016. The feature 2018 has also been updated to reflect one or more sleep or bedtime routines 2034 for the user. Refer to FIG. 20B for further discussion about putting the routines 2034 information to the user.

FIG. 20E illustrates the GUI 2030 that has been updated once the user selects the feature 2020 to view information about their day in FIG. 20C. As shown here, when the feature 2020 is selected, the GUI 2030 can output the feature 2016 and recommendations 2036. Refer to discussion about the panel 2002 in FIG. 20A for further discussion.

FIG. 21 is an example GUI 2100 for receiving input to a sleep health questionnaire from a user. As described herein, the user can complete a sleep health and wellness questionnaire (as shown in the GUI 2100) by providing subjective responses to questions about the user's sleep health. The questionnaire in the GUI 2100 can be accessed from a mobile application or webpage at the user's mobile device. For example, the user can view their wellness profile in the mobile application and select an option to respond to one or more questionnaires about their sleep, health, habits, and other conditions/factors. Responses that the user provides to the questionnaires can be used by a computer system described herein to generate curated insights and recommendations for improving the user's overall sleep quality and/or health.

The user can complete the questionnaire at any point in time. For example, the user can complete the questionnaire when they create an account or wellness profile with the mobile application (e.g., when the user sets up their bed system with a sleep service having the mobile application). The user may also access the questionnaire whenever they desire from the mobile application and answer the questionnaire and/or update their responses to the questionnaire. The user may desire, for example, to answer the questionnaire after waking up from a sleep session (e.g., immediately after, some amount of time after waking up). Sometimes, the mobile application can present prompts or notifications to the user that remind the user to complete the questionnaire, update their responses, and/or provide responses to questions in the questionnaire that the user has not responded to. These prompts can include in-app alerts or other types of notifications that can be presented at the user's mobile device.

The GUI 2100 can include a panel 2102, which presents information about one or more questionnaires that the user can complete. The panel 2102 can include a prompt indicating that the user should answer questions in one or more questionnaires in order to receive personalized tips that can improve the user's overall sleep health and/or quality. In the example of FIG. 21, selectable options 2104A-N are presented in the panel 2102. Each of the options 2104A-N corresponds to a questionnaire for the user to complete. User responses to each of the questionnaires can then be used by the computer system to determine one or more personalized insights to deliver to the user. When the user selects one of the options 2104A-N, the panel 2102 can be replaced with another panel, such as panel 2106. In other words, the GUI 2100 can be updated such that information presented in the panel 2102 is replaced by one or more questions corresponding to the selected option.

Each option 2104A-N can include a graphical element (e.g., icon) that visually depicts a type of questionnaire associated with selecting the option. Each option 2104A-N may include text indicating the type of the questionnaire, how many questions the questionnaire includes, and how many of those questions the user has completed. Accordingly, the user can easily identify which questionnaires they should complete.

In the illustrative example of FIG. 21, option 2104A corresponds to a sleeper questionnaire having 12 questions to be completed. Option 2104B corresponds to a home questionnaire having 5 questions to be completed. Option 2104C corresponds to a habits questionnaire having 9 questions to complete. Option 2104N corresponds to a sleep health questionnaire having 6 questions to complete. One or more other combinations of questionnaires and/or quantity of questions (additional or fewer questions) may be presented in the user's wellness profile in the GUI 2100.

Here, the user selects option 2104N to access the sleep health questionnaire described herein. Answers to the sleep health questionnaire can be used by the computer system to determine the user's circadian rhythm and circadian chronotype. As a result, the computer system can identify a curated set of personalized insights that correspond to the user's circadian chronotype, maintains the user's circadian rhythm, and/or improves the user's sleep health and/or quality over time.

When the user selects the option 2104N, the GUI 2100 can be updated to present the panel 2106. Each question in the sleep health questionnaire can be presented in the GUI 2100. Each question can correspond to a different factor that may be used, by the computer system, to determine the user's circadian chronotype, and consequently, personalized insights for the user. After all, each factor can impact sleep quality and may interact differently with the user's circadian rhythm and/or circadian chronotype. For example, regularity (e.g., routinely going to bed around the same time, waking up around the same time), duration (e.g., how long the user sleeps), efficiency (e.g., how well the user sleeps during a sleep session), timing (e.g., consistency in sleep timing, constant bedtime routine, tied with regularity), satisfaction (e.g., subjective feelings of whether the user slept well and feels refreshed after a sleep session), and alertness (e.g., subjective perception of how alert the user is during the day or between consecutive sleep sessions) are some factors that may impact sleep quality and interact differently with the user's circadian rhythm and/or circadian chronotype. The sleep health questionnaire presented in the GUI 2100 can pose questions directed to one or more of these factors.

The computer system can then generate a set of personalized insights and tips that are specific to the factors that the user answered questions about. Therefore, the computer system can use the user's answers to the questions to identify one or more insights to present to the user. In some implementations, the computer system may also leverage system-based inferences and/or sensed data from one or more sensors of a bed system to identify and curate what insights to present to the user. For example, the computer system can determine a sleep score for the user after a most recent sleep session using any of the techniques previously described. The computer system can then use the sleep score in combination with the user's responses to the sleep health questionnaire to identify one or more insights directed to the factors mentioned above for presentation to the user. Sometimes, the user's sleep score can lower over time if the user's circadian rhythm is off. Because the user's circadian rhythm is off, any one or more of the factors of sleep regularity, duration, efficiency, timing, satisfaction, and alertness may be negatively impacted. Accordingly, the computer system can identify and/or generate one or more insights or tips about how the user's circadian rhythm is impacting the factor(s) to gently nudge the user to take action that gets their circadian rhythm back on track and thus improves the particular factor(s).

As an illustrative example, if the user is a night owl and answered an efficiency question by identifying that they struggle to fall asleep and/or stay asleep, the computer system can identify one or more tips for night owls that have the same/similar difficulty and randomly present those tips to the user at the user's mobile device. Such tips can be presented as an in-app notification when the user opens the mobile application. Such tips can be presented when the user selects an option to view details about their most recent/last sleep session in the mobile application. Such tips may also be randomly presented in one or more other GUIs in the mobile application, as determined by the computer system. As described herein, the tips can also be presented in the GUIs based on applying one or more intelligent machine learning rules logic.

Still referring to FIG. 21, the panel 2106 can prompt the user to answer one or more questions about the user's perceived sleep health so that the user can receive tips on improving their sleep health.

A first question 2108 can ask the user whether they are an early bird or a night owl. The user's response to the question 2108 can be used, by the computer system, to determine the user's circadian rhythm and/or circadian chronotype. The computer system can also use the user's response to identify and provide insights corresponding to the user's circadian chronotype. The user can answer the question 2108 by selecting an option from a drop-down menu. The drop-down menu can include, for example, selectable options for a night owl, early bird, and neutral. If the user selects an option such as neutral, then the computer system may not use the user's response to the question 2108 for identifying and generating a curated set of personalized insights for the user. Sometimes, if the user selects the option for neutral, the computer system can identify general insights that may pertain to users who are neither night owls nor early birds.

A second question 2110 can ask the user whether any one or more factors disrupt the user's sleep on a regular basis. The user may select any one or more selectable options presented with the question 2110 that correspond to factors that may disrupt their sleep. For example, the user may select options for stress/worry, pain, temperature (too hot or too cold), allergies, tossing and turning, partner's tossing and turning, snoring, partner snoring, none of the above. The computer system can identify and provide one or more insights to present to the user that correspond to the options that the user selects.

A third question 2112 can ask the user how many days a week they have one or more identified sleep problems. The question 2112 can identify one or more sleep problems for which the user can provide answers. For example, the sleep problems can include, but are not limited to, trouble falling asleep, waking up during the night, returning to sleep after waking during the night, and/or waking up too early and being unable to fall back asleep. For each of the identified sleep problems, the user can select an option for 0 days a week, 1-2 days a week, 3-4 days a week, 5+ days a week. As described herein, the computer system can use the user's responses to the question 2112 to identify and provide insights about how to improve any of these problems. The insights selected may also vary based on the frequency that the user experiences these problems (e.g., the more often the user has trouble falling asleep, the greater quantity tips identified that address this issue).

A fourth question 2114 can ask the user whether they have been diagnosed as having one or more identified conditions. The user may select any one or more selectable options presented with the question 2114 that correspond to the identified sleep conditions. The sleep conditions can include, but are not limited to, sleep apnea (breathing disruptions), hypersomnia or narcolepsy (extreme sleepiness), insomnia (problems falling asleep or staying asleep), restless leg syndrome (urge to move legs), parasomnia (move around, talk, or do unusual things during sleep), and none of the above. The computer system can use the user's responses to the question 2114 to identify and provide insights about how to mitigate and of these conditions (assuming the user indicates that they have been diagnosed with the condition(s)).

A fifth question 2116 can ask the user how often, if ever, they use a CPAP machine. The user can select an option from a drop-down menu in response to the question 2116. The drop-down menu can include options for varying frequencies of using a CPAP machine. The user's response to the question 2116 can be used by the computer system to identify and provide insights about how to improve the user's sleep quality and/or health.

A sixth question 2118 can be presented in the GUI 2100 that asks the user how often they currently use any one or more identified sleep aids. The user may select an option for each of the identified sleep aids. The options can include rarely or never, occasionally (once a month), and regularly (once a week). The identified sleep aids can include, but are not limited to, prescribed sleep medications, over-the-counter sleep medications, natural remedies (e.g., melatonin), drug free sleep aids (white noise, meditation, music, etc.), and sleep coaching or sleep behavioral programs. The computer system can use the user's responses to the question 2118 to identify and provide insights that may help the user reduce dependency or need for any of the identified sleep aids.

The questions 2108-2118 can be presented in any order. In some implementations, one or more additional or fewer questions may be presented in the sleep health questionnaire.

Once the user answers any of the questions 2108-2118, the user can select an option (e.g., button) 2120 to save their responses. Selecting the option 2120 may cause the mobile device to transmit the user's responses to the computer system (e.g., over a network) for storage and further processing. For example, the computer system can now use the user's responses to identify and generate a curated set of insights for the user. Moreover, the computer system can identify a curated set of insights for the user based on any combination of user responses to the questions 2108-2118. For example, the computer system can select insights corresponding to the user's answers to all of the questions 2108-2118. Sometimes, the computer system may select insights corresponding to only some of the user's answers to the questions 2108-2118.

Although the GUI 2100 depicts the features 2102, 2106, 2108, 2110, 2112, 2114, 2116, and 2118 in one GUI 2100, one or more of these features can be displayed at once. In other words, once the user provides user input in response to a question in one of the features, the feature can be replaced in the GUI 2100 with another feature. As another example, once the user provides user input to a question in one feature, the GUI 2100 can be set into motion and a next feature can appear from a bottom of the screen and put the one feature up towards a top of the screen until the one feature is partially presented in the GUI 2100 or no longer presented. As yet another example, once the user provides user input to a question in one feature, the user can scroll down to view a question in a next feature. The user can continue to scroll through all the features until the user responds to the questions presented in the features.

When the user completes the questionnaire they can respond to as many of the questions posed in the questionnaire as they desire. For example, the user can respond to all of the questions. The user may also respond to less than all of the questions. Regardless of how many questions the user responds to, the computer system described herein can still identify a curated set of insights for the user that correspond to only the answered questions. For example, if the user does not respond to a question about their subjective stress level during the day, then the computer system may not identify insights to present to the user that correspond to dealing with stress levels. The user may also return to the questionnaire at a later time and update or modify one or more of their responses. For example, the user can remove one or more prior responses. The user can respond to a question they did not previously respond to. The user can also update their response to a question they previously responded to. Whenever the questionnaire responses are updated by the user, the computer system can update the curated set of insights for the user so that insights most relevant to the user's current subjective beliefs about their sleep health are accounted for.

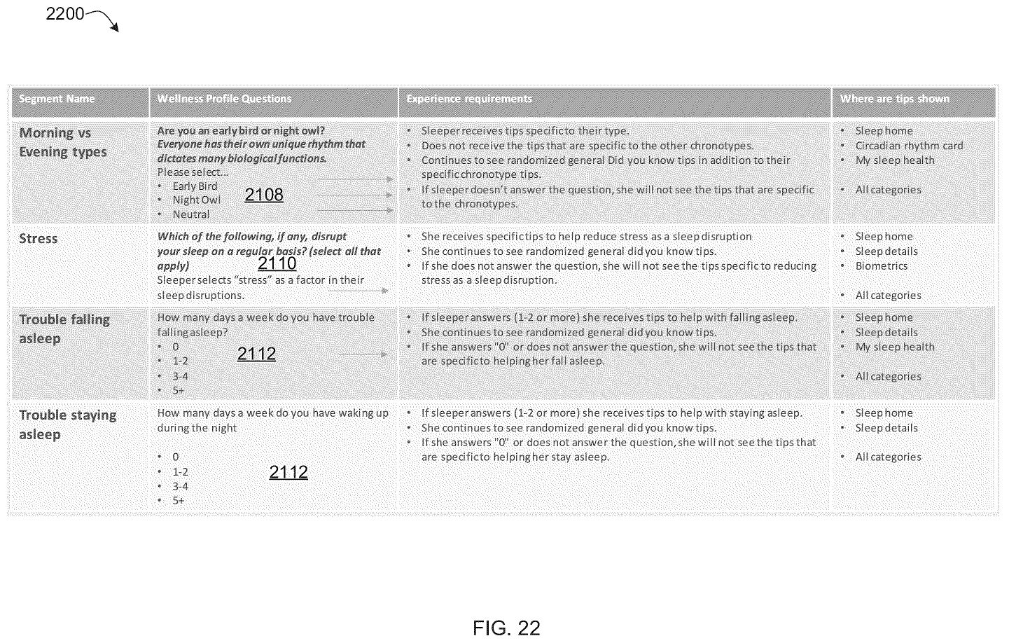

FIG. 22 is a block diagram of a table 2200 describing example parameters for generating personalized insights based on user input to a sleep health questionnaire. More specifically, the table 220 indicates how different user responses to the questionnaire described in FIG. 21 can be used by a computer system to identify insights to present to the user.

For example, the question 2108 in FIG. 21 about whether the user is an early bird or night owl corresponds to an insight segment of morning versus evening types. The user can respond by selecting options for early bird, night owl, or neutral. The computer system can select and identify tips specific to whether the user is a morning type or an evening type. The computer system may not select or identify tips that are specific to other chronotypes (e.g., if the user is a morning type, then the user may not receive tips that correspond to the evening type). In some implementations, the computer system may also continue to randomize selection and presentation of general “Did you know” tips to the user in addition to the tips that are specific to the user's chronotype (e.g., morning type or evening type). If the user does not answer the question 2108, then the computer system may not identify or present tips that are specific to either chronotype, morning or evening type. The tips selected in response to the question 2108 can be presented in a variety of GUIs in a mobile application at the user's mobile device. For example, the tips can be presented in a home screen of the mobile application. The tips can be additionally or alternatively presented in a GUI displaying information about the user's circadian rhythm. The tips can be additionally or alternatively presented in a GUI displaying information about the user's sleep health. The tips can be additionally or alternatively presented in one or more other GUIs presented in the mobile application, regardless of whether the GUIs pertain to the circadian rhythm of the user.

The question 2110 in FIG. 21 about whether any identified problems regularly disrupt the user's sleep can correspond to an insight segment for stress. The question 2110 may also correspond to other insight segments including but not limited to pain, temperature, allergies, tossing and turning, partner's tossing and turning, snoring, and/or partner snoring. As shown in the table 2200, when the user selects stress as a factor that disrupts their sleep in response to the question 2110, the computer system can identify and select tips specific to reducing stress as a sleep disruption. The computer system may also continue to select and randomly present general “Did you know” tips to the user in combination with the stress reduction tips. If the user does not respond to the question 2110, then the computer system may not identify and present tips specific to reducing stress. The tips selected in response to the question 2110 can be presented in a variety of GUIs in the mobile application at the user's mobile device. For example, the tips can be presented in a home screen of the mobile application. The tips can be additionally or alternatively presented in a GUI displaying information about the user's sleep details (e.g., a prior, last or most recent sleep session, a history of one or more consecutive sleep sessions). The tips can be additionally or alternatively presented in a GUI displaying information about the user's biometrics. The tips can be additionally or alternatively presented in one or more other GUIs presented in the mobile application, regardless of whether the GUIs pertain to stress or other sleep disruption factors.

The question 2112 in FIG. 21 about how many days a week the user has trouble falling asleep can correspond to an insight segment for trouble falling asleep. The question 2112 may also correspond to other insight segments including but not limited to how many days a week the user wakes up during the night, has trouble returning to sleep after waking up during the night, and/or wakes up too early and cannot fall back asleep. As shown in the table 2200, when the user selects any of the selectable number of days a week that the user has trouble falling asleep, the computer system can identify and select tips specific to help the user fall asleep. For example, if the user answers 1-2, 3-4, or 5+ days a week, then the computer system can select and present tips specific to help the user with falling asleep. The computer system may also continue to select and randomly present general “Did you know” tips to the user in combination with the help with falling asleep tips. If the user does not respond to the question 2112 with regards to trouble falling asleep (or selects 0 days), then the computer system may not identify and present tips specific to help with falling asleep. The tips selected in response to the question 2112 can be presented in a variety of GUIs in the mobile application at the user's mobile device. For example, the tips can be presented in a home screen of the mobile application. The tips can be additionally or alternatively presented in a GUI displaying information about the user's sleep details (e.g., a prior, last or most recent sleep session, a history of one or more consecutive sleep sessions). The tips can be additionally or alternatively presented in a GUI displaying information about the user's sleep health. The tips can be additionally or alternatively presented in one or more other GUIs presented in the mobile application, regardless of whether the GUIs pertain to trouble with falling asleep.

The question 2112 in FIG. 21 can also ask the user how many days a week the user wakes up during the night, which can correspond to an insight segment for trouble staying asleep. As described above, the question 2112 may also correspond to other insight segments. As shown in the table 2200, when the user selects any of the selectable number of days a week that the user has trouble staying asleep, the computer system can identify and select tips specific to help the user with staying asleep. For example, if the user answers 1-2, 3-4, or 5+ days a week, then the computer system can select and present tips specific to help the user with staying asleep. The computer system may also continue to select and randomly present general “Did you know” tips to the user in combination with the help with staying asleep tips. If the user does not respond to the question 2112 with regards to trouble staying asleep (or selects 0 days), then the computer system may not identify and present tips specific to help with staying asleep. The tips selected in response to the question 2112 can be presented in a variety of GUIs in the mobile application at the user's mobile device. For example, the tips can be presented in a home screen of the mobile application. The tips can be additionally or alternatively presented in a GUI displaying information about the user's sleep details (e.g., a prior, last or most recent sleep session, a history of one or more consecutive sleep sessions). The tips can be additionally or alternatively presented in one or more other GUIs presented in the mobile application, regardless of whether the GUIs pertain to trouble with falling asleep.

Although the table 2200 indicates only some example tip behaviors based on user responses to the questions posed in the sleep health questionnaire, one or more other similar tip behaviors may exist for user responses to the other questions posed in the sleep health questionnaire.

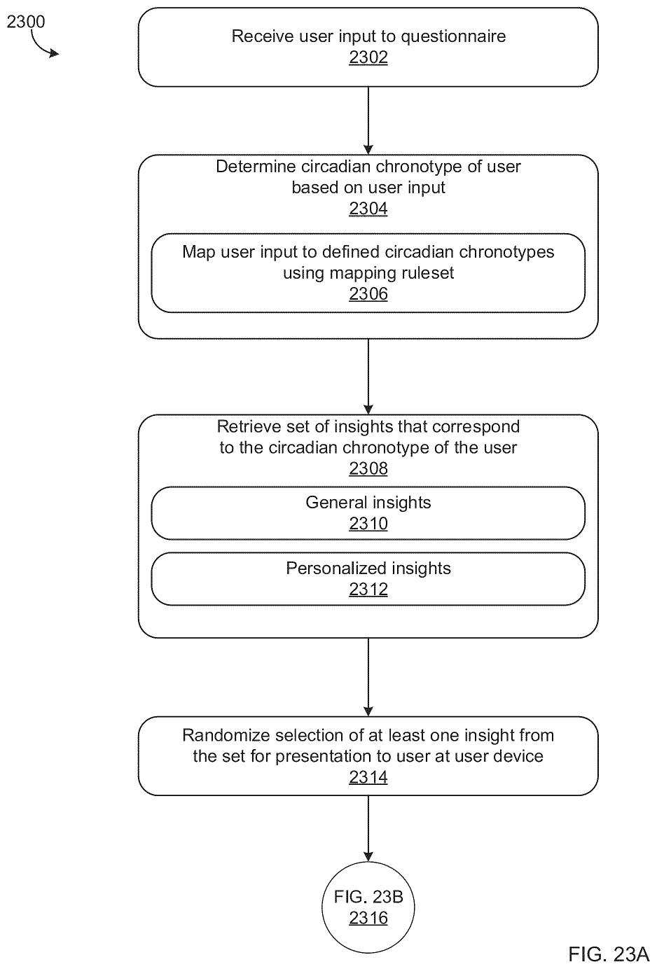

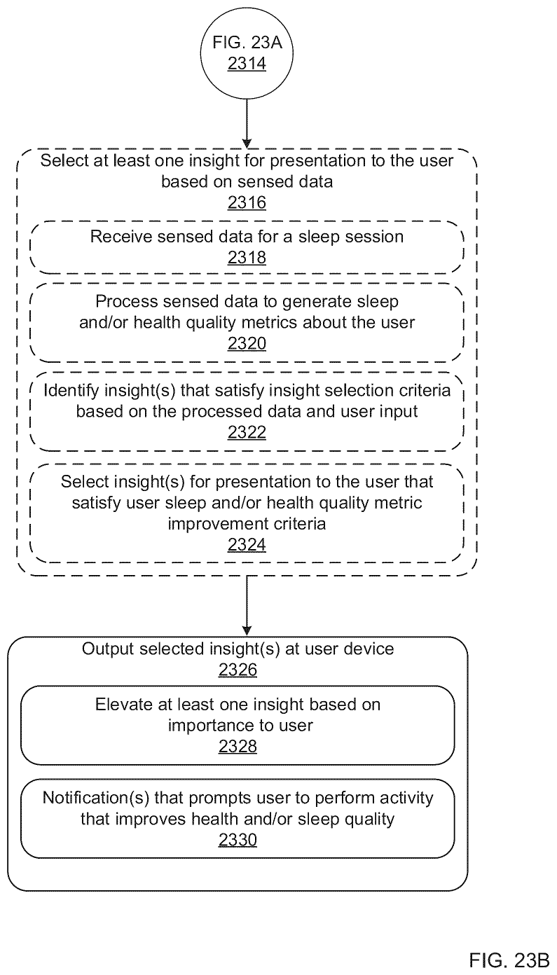

FIGS. 23A-B is a flowchart of process 2300 for determining personalized insights for a user. The process 2300 can be performed by the computer system 1902. The process 2300 can also be performed by one or more other computing systems, devices, computers, networks, cloud-based systems, and/or cloud-based services. The process 2300 can be performed by a cloud computing system, a user device of the user, and/or a home automation hub or other home automation device. For illustrative purposes, the process 2300 is described from the perspective of a computer system.

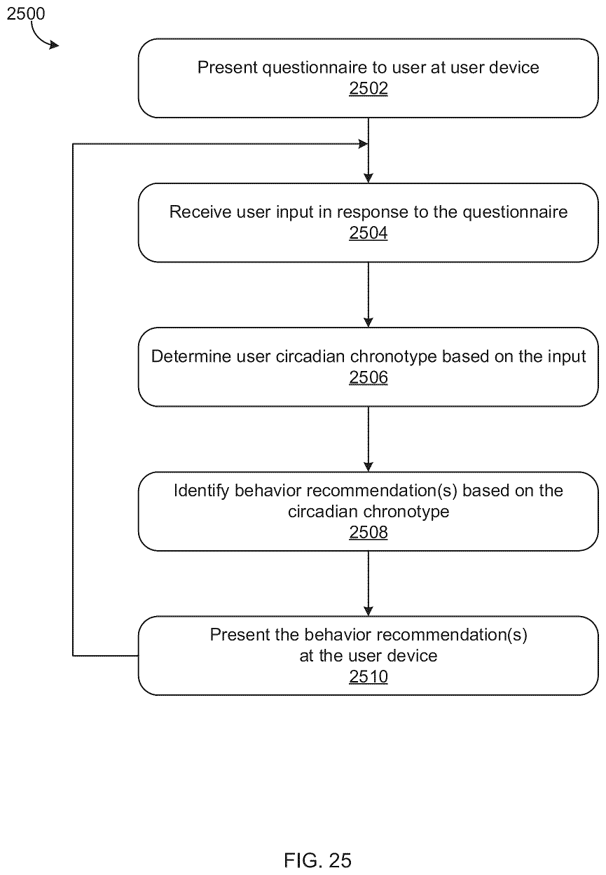

Referring to the process 2300 in both FIGS. 23A-B, the computer system can receive user input to a questionnaire in block 2302. For example, the computer system can transmit, to the user device, a subjective sleep health questionnaire for presentation in a graphical user interface (GUI) display at the user device. The questionnaire can be presented in a mobile application, webpage, or web browser loaded at the user device. Sometimes, the computer system can transmit the subjective sleep health questionnaire to the user device based on receiving an indication, from the user device, that the user registered an account with the computer system (e.g., created a sleep profile, set up or initialized a bed system). Transmitting, to the user device, the subjective sleep health questionnaire for presentation in the GUI display can be performed a single instance. For example, the user can be notified to complete the questionnaire when they load up the mobile application after registering with the computer system. The user can then decide to complete the questionnaire at that time or at another time. The computer system may not notify the user again to complete the questionnaire. In some implementations, the computer system may notify the user at predetermined time intervals (e.g., 3 days after the first notification and the user has not yet completed the questionnaire, 5 days after, 7 days after) until the user completes the questionnaire. Accordingly, the questionnaire can be presented once at the user device. The questionnaire may also be completed once, although the user can go back and update, modify, or change any of their responses to the questionnaire at later times. Refer to FIG. 25 for further discussion.

The subjective sleep health questionnaire can be presented in a first screen in the GUI display. Any insights, behavior recommendations, reminders, or tips that are generated by the computer system can be presented in a second screen in the GUI display, the second screen being different than the first screen. Sometimes, as described herein, the generated/identified insights can be presented as notifications, pop-ups, messages, or alerts in one or more other screens in the GUI display. For example, a tip about winding down earlier than when the user typically has winded down before bedtime can be presented in any screen that the user is currently on/viewing at their user device. The tip can also be presented in one or more other screens that may present similar or associated information with the tip (e.g., a screen that shows a summary of the user's most recent sleep session).

Still referring to block 2302, the computer system can receive, from the user device, user input indicating at least one response to the subjective sleep health questionnaire. The user may desire to answer all or some questions in the questionnaire. Any user response, whether alone or in combination, can then be used by the computer system to determine a circadian chronotype of the user and/or tips/insights/reminders/recommendations to present to the user. As described herein, the computer system can also receive, from the user device, updated user input to the subjective sleep health questionnaire at a later time than when the computer system received the user input. The later time can be any amount of time after the user completed the questionnaire a first time. For example, the user can complete the questionnaire on a same day that the user creates an account with the computer system. Several days after completing the questionnaire, the user may decide that their responses were inaccurate. The user can navigate back to the questionnaire in the mobile application at their user device and update one or more of their responses. The updated responses can then be received by the computer system to perform the techniques described herein.

The questionnaire, as described herein (e.g., refer to FIG. 21), can have questions about the user's circadian rhythm, user-perceived circadian chronotype (e.g., night owl, early bird, neutral), user-perceived stress levels, user-perceived trouble falling asleep, user-perceived trouble staying asleep, etc. The questionnaire can prompt the user for user input about at least one of the user-perceived factors described above. As described further below, the computer system may also retrieve and process historic sleep data associated with the user to verify and/or validate the user responses to the user-perceived factors. By doing so, the computer system can more accurately determine the circadian chronotype of the user and generate/identify insights, tips, recommendations, and/or reminders to present to the user based on their circadian chronotype.

As described in reference to FIG. 21, the subjective sleep health questionnaire can prompt the user for an indication of whether the user is an early bird, a night owl, or neutral. The subjective sleep health questionnaire can prompt the user for an indication of at least one factor that disrupts sleep patterns of the user. The subjective sleep health questionnaire can prompt the user for an indication of frequency of at least one sleep problem. The at least one sleep problem can include at least one of the group including trouble falling asleep, waking up during a sleep session, returning to sleep after waking up during the sleep session, and/or waking up too early and being unable to fall back asleep. Sometimes, the subjective sleep health questionnaire can prompt the user for an indication of at least one sleep condition for which the user has been or may be diagnosed with. The subjective sleep health questionnaire may also prompt the user for an indication of how frequently the user uses at least one sleep-aid device, including but not limited to CPAP devices. One or more other questions can be presented to the user in the questionnaire, as described herein.

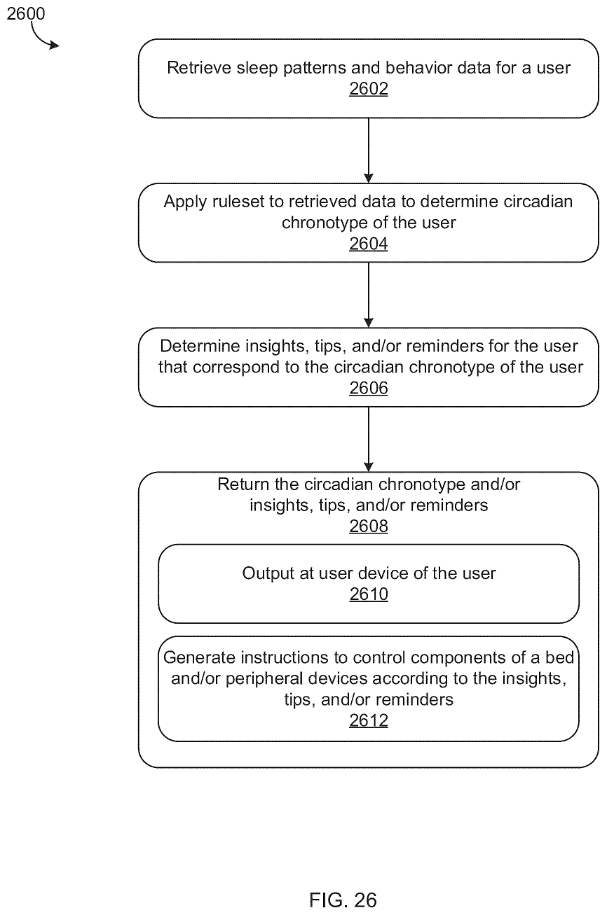

In block 2304, the computer system can determine a circadian chronotype of the user based on the user input. The computer system can process the user input. For example, the computer system can map the user input to defined circadian chronotypes using a mapping ruleset (block 2306). In other words, the computer system can retrieve, from a data store, a lookup table that maps responses to the subjective sleep health questionnaire to circadian chronotypes, and select the circadian chronotype that matches the at least one response from the user. The computer system can also retrieve other criteria, conditions, and/or rulesets from the data store, which can then be used to determine which user responses and/or combination of responses corresponds to which circadian chronotype (e.g., night owl, early bird, or neutral). In some implementations, the computer system can also determine circadian chronotype based on data sensed and/or collected by sensors of a bed system during sleep sessions of the user (e.g., refer to FIG. 19) and/or historic sleep data associated with the user (e.g., refer to FIGS. 26-28). In some implementations, the computer system can determine the circadian chronotype of the user based on comparing the user responses to the questionnaire with historic sleep data and circadian chronotypes of a general population of users that are similar to the user (e.g., similar age, gender, geographic location, demographics). Sometimes, the computer system can apply one or more machine learning trained models to the user input to determine the circadian chronotype. The model(s) can be trained to map/correlate various user responses and/or combinations of user responses with one or more different circadian chronotypes. The model(s) can also generate confidence values indicating likelihood that the user is associated with one or more of the circadian chronotypes. If the model determines that the user is associated with more than one circadian chronotype (e.g., both neutral and early bird), then the computer system can determine that the user is associated with the circadian chronotype having the highest confidence value (or a confidence value within a threshold range or exceeding a threshold value/range).

In block 2308, the computer system can retrieve a set of insights from a data store that correspond to the determined circadian chronotype of the user. In other words, the computer system can identify, based on the circadian chronotype of the user, at least one insight for presentation in the GUI display at the user device. The insights can include tips, reminders, behavior recommendations, or other types of notifications described throughout this disclosure. The tips can be generated by the computer system at another time (e.g., before the user or other users complete the questionnaire) and stored in the data store for quick identification and retrieval during runtime use of the disclosed technology. The computer system can generate the tips based on processing and analyzing robust sets of historic data for populations of users and/or individual users. The tips can then be bucketized and associated with different circadian chronotypes. During runtime, the computer system may simply identify the tips associated with the particular circadian chronotype of the user and present those tips to the user. The computer system may not need to employ additional compute resources and/or processing power to generate insights during runtime. The compute resources and/or processing power may instead be put to use in performing other computationally heavy processes both efficiently and accurately.

In some implementations, computer system may generate one or more insights for the particular user during runtime use of the disclosed technology. The computer system may use templates to generate the insights, the templates corresponding to the determined circadian chronotype of the user. For example, the computer system can retrieve, from the data store, one or more insight templates that are associated with the circadian chronotype of the user. The computer system can then plug particular information into the template that is relevant to the particular sleep behavior, habits, or other user/sleep/health data of the user. This template insight can be presented to the user. This template insight can also be stored and later retrieved and presented to the user. In some implementations, this template insight may also be used and presented to other users who have the same circadian chronotype, similar demographics, and/or similar user/sleep/health data as the user.

The computer system can retrieve and/or generate general insights (block 2310). For example, to select at least one insight from a subset of insights, the computer system can assemble the subset of insights from a template of general insights that have been completed with information generic to a population of users that include the user. At least one insight presented to the user can be a general, wellness education insight that corresponds to sleep and health data of a general population of users that includes the user. The general insights can provide general education tips for the population of users who have the same circadian chronotype and/or similar data. An example general insight can include general information about what it means to be a night owl or an early bird.

The computer system can additionally or alternatively retrieve and/or generate personalized insights (block 2312). For example, to select at least one insight from a subset of insights, the computer system can assemble the subset of insights from a template of personalized insights that have been completed with information specific to the user. The personalized insight can correspond to historic sleep and health data associated with the particular user. The personalized insight can be generated from a template insight, as described above. The personalized insight may be a wellness insight and/or personalized recommendation (e.g., suggestion of when to go to sleep, when to wind down for the night, when to eat a meal such as dinner, when to exercise) based on the user's particular data. As an illustrative example, the personalized insight can indicate that since the user is a night owl, they should go to bed 30 minutes earlier each night otherwise they may feel more tired in the morning when they wake up.

One or more other general and/or personalized insights can be identified for the particular user. Furthermore, the computer system can determine any combination of general and/or personalized insights to identify, generate, and/or present to the user.

In block 2314, the computer system can randomize selection of at least one insight from the set of insights for presentation in the GUI display at the user device of the user. Selection of the insight for presentation can be randomized such that every day or other predetermined time period the user may see one or more different insights. Therefore, the presentation of insights can be engaging while also gently nudging the user to improve, modify, or adjust their sleep behavior and/or habits. As described throughout this disclosure, the computer system can additionally or alternatively use intelligent machine learning rules and logic to select the at least one insight for presentation in the GUI display.

As described herein, the at least one insight can be one or more different types of notifications and/or messages for the user. For example, the at least one insight can be a behavior recommendation to maintain a current circadian rhythm of the user. The at least one insight can be a behavior recommendation to improve a current circadian rhythm of the user over a predetermined period of time.

The predetermined period of time can be a next sleep session (e.g., the next time the user goes to sleep for the night). The predetermined period of time can be a threshold quantity of successive sleep sessions. The sleep sessions can be determined using a variety of techniques. The sleep sessions can be determined using a clock and/or calendar to identify when the user goes to sleep and when they wake up from that sleep. For example, if the user goes to sleep at 8 PM on a Tuesday, then 1 full sleep session can be counted from that 8 PM bedtime on Tuesday to an 8 PM bedtime on the following day, Wednesday. Accordingly, 2 successive sleep sessions would extend from 8 PM on Tuesday to 8 PM on Thursday. This technique for determining sleep sessions can be useful with bed systems that may not have presence detection techniques.

As another example, the sleep sessions can be determined by detecting presence of the user on the bed (e.g., using pressure sensors, temperature sensors, and/or other sensing devices/modalities of the bed system as described above) and counting instances of when the user lays in the bed, falls asleep, remains asleep, remains in bed, wakes up, and/or gets out of bed. This technique can be useful in scenarios where the user typically sleeps in their own bed and the bed has the necessary devices/components to track/monitor movement/presence of the user on the bed. This technique may also be useful in scenarios in which the user typically experiences disrupted sleep sessions (e.g., due to work, other stresses in life, taking naps).

The threshold quantity can be 5 successive sleep sessions (e.g., 5 following nights of sleep). The threshold quantity can be 7 successive sleep sessions (e.g., 7 following nights of sleep). One or more other predetermined periods of time can be used. For example, the predetermined periods of time can include week days or a subset of week days. The predetermined periods of time can include weekends and/or a combination of week days and weekends.

In some implementations, the at least one insight can be a behavior recommendation to reduce a current level of stress of the user by a threshold amount. The at least one insight can be a behavior recommendation to improve an ability of the user to fall asleep faster by a threshold amount. The threshold amounts described herein can be determined by the computer system and based on analysis of historic sleep and/or health data of the user. The computer system can generate more subtle recommendations (e.g., a lower threshold amount), for example, based on determining that the user has experienced low levels of stress over time and/or sporadic difficulties with falling asleep over time. As another example, the computer system can generate more extreme recommendations (e.g., a higher threshold amount) based on determining that the user has experienced consistent and/or higher levels of stress over time and/or more frequent difficulties falling asleep.

As another example, the at least one insight can be a behavior recommendation to improve an ability of the user to stay asleep during a sleep session. The at least one insight may also include a recommendation of when the user should exercise. The at least one insight may include a recommendation of when the user is most likely alert during a predetermined period of time. The predetermined period of time can be an amount of time between two successive sleep sessions, as described above. The predetermined period of time can be an amount of time between when the user wakes up from a first sleep session and when the user goes to sleep in a second sleep session. The predetermined period of time can be one or more other periods of time as described above.

As more examples, the at least one insight can include a recommendation of when the user should eat a meal. The insight can include a recommendation of when the user should begin a bedtime routine. Starting the bedtime routine can include winding down before going to sleep, such as brushing teeth, showering, turning off electronic devices, dimming or turning off lights, getting into bed, reading, etc. The at least one insight can include a notification prompting the user to perform an activity that improves at least one of a current health metric and a current sleep quality of the user. The activity can include, but is not limited to, using one or more features of the user's bed system to improve their overall sleep health and/or sleep quality (e.g., turning on a responsive air feature, changing a firmness setting, adjusting positions of a base of the bed system), ways to wind down before going to sleep, importance of improving sleep quality to improve the user's overall health, ways to improve restful sleep of the user, etc.

Moreover, the at least one insight can include a tip about the particular circadian chronotype of the user and at least one behavior recommendation to improve the sleep quality or health metric of the user or otherwise maintain the circadian rhythm of the user that corresponds to the circadian chronotype. As shown and described in FIGS. 20A-B, the tip and the at least one behavior recommendation can be presented concurrently in the GUI display, the GUI display being scrollable in a vertical direction. In response to receiving user input indicating a scrolling feature, the user device can present at least a portion of the tip and the at least one behavior recommendation concurrently in the GUI display. Another portion of at least one of the tip and the at least one behavior recommendation may no longer visible in the GUI display, in some implementations.

Sometimes, the at least one insight further can include a selectable option to generate daily routines based on the at least one behavior recommendation. The selectable option can be presented concurrently in the GUI display with the tip and the at least one behavior recommendation. In response to receiving user input indicating selection of the selectable option, the user device can present another screen in the GUI display that may replace the presentation of the tip, the at least one behavior recommendation, and the selectable option.

In some implementations, as shown and described in reference to FIGS. 20A-B, the at least one behavior recommendation can be presented in the GUI display with a graphical element corresponding to a type of the at least one behavior recommendation. The type of the at least one behavior recommendation can be, as described above, a time-to-exercise recommendation and the corresponding graphical element can be exercise equipment, the type of the at least one behavior recommendation can be a most-alert recommendation and the corresponding graphical element can be a lightbulb, the type of the at least one behavior recommendation can be a time-to-eat recommendation and the corresponding graphical element can be a utensil, and the type of the at least one behavior recommendation can be a time-to-prepare-for-bed recommendation and the corresponding graphical element can be a coffee cup. Refer to FIGS. 20A-B for further depiction and discussion. Sometimes, the at least one behavior recommendation can be presented in the GUI display with an indication of a threshold period of time for performing the at least one behavior recommendation. As described above, the threshold period of time can indicate a range of time at which performing the at least one behavior recommendation can improve at least one of the sleep quality and the health metric of the user.

In some implementations, the computer system can identify the at least one insight in block 2314 based on retrieving, from the data store, sleep and health insights associated with the circadian chronotype of the user, identifying, based on the processed user input, a subset of insights from the retrieved sleep and health insights, and selecting, using a randomization technique, the at least one insight from the subset of insights for presentation to the user. The randomization technique can be a random number generator or any other type of randomization algorithm, ruleset, and/or technique. As described herein, the computer system can additionally or alternatively use machine learning rules to select the at least one insight. For example, machine learning rules and logic can leverage sensor data and historic sleep data of the user to deliver unique insights to the user each day and/or at predetermined time intervals (e.g., after a sleep session, after multiple sleep sessions). Sometimes, the user may receive repeated insights, but those insights can be managed over time using one or more machine learning rules and/or prioritization techniques described herein.

One or more of the blocks 2302-2312 can occur once. For example, the blocks 2304-2312 can be performed once, in response to the user completing the questionnaire a first time in block 2302. One or more of the blocks 2304-2312 can then be performed one or more additional times to update the circadian chronotype and/or set of insights for the user whenever the user provides updated user input to the questionnaire in block 2302 (or new patterns and/or behavior are detected in the historic sleep data of the user).

Moreover, block 2314 can be performed once (e.g., with the blocks 2302-2312). For example, once the set of insights are retrieved/determined in block 2308, the computer system can randomly determine an order by which to present each of the insights in the set to the user (block 2314). Then, whenever the user opens a particular screen in the mobile application and the user device and/or a predetermined time interval passes (e.g., after the user wakes up from a sleep session, once a day, in the morning, in the evening, whenever the user logs into/accesses the mobile application), the computer system can deliver the insight next in order to the user device for presentation at the user device.

Sometimes, block 2314 can be performed at predetermined time intervals, such as once a day (e.g., when the user wakes up from a sleep session, before the user goes to sleep), every few days, every few sleep sessions, etc. For example the set of insights can be retrieved and/or determined in block 2308 once, when the user provides responses to the questionnaire. Then, every day, every few days, every few sleep sessions, or during any other predetermined time interval (e.g., whenever the user opens the mobile application at the user device, whenever the user opens the mobile application a predetermined amount of time after a last time the user opened the mobile application), the computer system can randomize selection of the insight from the set of insights to present to the user in block 2314.

Still referring to the process 2300 in FIGS. 23A-B, the computer system can optionally select at least one insight for presentation to the user based on sensed data at the bed system during one or more previous sleep sessions of the user (block 2316). Therefore, the computer system can determine what insights to present to the user that maintains their circadian rhythm and also relates to how the user slept during a previous sleep session. This insight can be more personalized for the user and can gently nudge the user to actively make changes to their sleep habits.

For example, the computer system can optionally receive sensed data for a sleep session in block 2318. As described herein, the computer system can receive, from at least one sensor, sensor readings during a sleep session of the user. The sensed data can be received from sensors of a bed system, sensors in communication with components of the bed system, and/or wearable devices of the user, as described throughout this disclosure. The sensed data can include sleep information and/or health information about the user, such as their heartrate, respiration rate, etc.

The computer system can optionally process the sensed data to generate sleep and/or health quality metrics for the user in block 2320. In other words, the computer system can process the sensor readings to determine at least one of an objective sleep quality and an objective health metric. The sensed data can be processed using any of the techniques described above. For example, the sensed data can be processed to determine an overall sleep quality score for the user's previous sleep session.

The computer system can then optionally identify at least one insight that satisfies insight selection criteria based on the processed data and the user input in block 2322. For example, the computer system can identify a subset of insights based at least in part on the processed user input and the processed sensor readings. The insight selection criteria can indicate one or more factors relevant for presenting and/or elevating insights at the user device of the user. The criteria can additionally or alternatively include one or more threshold values to be satisfied in order to present a corresponding insight to the user. As an illustrative example, an insight can satisfy the criteria if the insight corresponds to a sleep quality score that is less than a threshold value (e.g., 50) and a circadian chronotype of a night owl. As another example, an insight can satisfy the criteria if the insight corresponds to a user snoring during a previous sleep session and a circadian chronotype of an early bird. As yet another example, an insight can satisfy the criteria if the insight corresponds to a user going to bed later than usual over one or more previous sleep sessions, the user feeling less alert during the day, and a circadian chronotype of a night owl.

The computer system can optionally select the insight for presentation to the user that satisfies user sleep and/or health quality metric improvement criteria in block 2324. In some implementations, the criteria in block 2324 can be the same as the criteria in block 2322. The criteria in blocks 2324 and 2322 can also be different. As an illustrative example, the insight selected in block 2324 can satisfy the criteria of block 2324 if the insight may cause the user to change their habits and thus increase their overall sleep quality score by a threshold amount. As another example, the insight selected in block 2324 can satisfy the criteria of block 2324 if the insight may cause the user to change their habits and thus feel more alert during the day/in between sleep sessions. As yet another example, the insight selected in block 2324 can satisfy the criteria of block 2324 if the insight may cause the user to change their habits by going to sleep earlier and/or around a same time over successive sleep sessions. In other words, the insight that satisfies the criteria in block 2324 can be selected by the computer system because it likely can be most effective in nudging the user to change their habits and improve their overall sleep quality and/or health.

In some implementations, the computer system may not perform block 2322 based on the sensed data. The computer system can identify insights that correspond to the user's circadian chronotype in block 2308. Then, once the computer system receives sensed data for a sleep session in block 2318, the computer system can select the at least one insight from the identified insights based at least in part on processing the sensor data (block 2324). Therefore, the computer system can leverage sensed data during one or more sleep sessions to select insights for presentation, where those insights have already been identified for the user based on the user's circadian chronotype. In some implementations, the computer system can leverage the sensed data to randomly select the insights for presentation.

In some implementations, the computer system can receive, from the at least one sensor, sensor readings during one or more sleep sessions of the user. The computer system can then process the sensor readings to determine a proposed circadian chronotype of the user. The computer system can then validate the proposed circadian chronotype of the user with the circadian chronotype that is determined based on the user input in block 2304. If the proposed circadian chronotype is validated with the determined circadian chronotype, then the computer system can proceed with blocks 2308-2314 in the process 2300. As another example, the computer system can validate the proposed circadian chronotype of the user by presenting the questionnaire to the user and receiving user input to the questionnaire in block 2302. Then, the computer system can determine a circadian chronotype of the user in block 2304 based on validating the proposed circadian chronotype with the user's answers to the questionnaire. Refer to FIGS. 26-28 for further discussion about determining the proposed circadian chronotype and/or validating the proposed circadian chronotype.

The computer system can output the selected insight(s) at the user device in block 2326. In scenarios in which any of the blocks 2316-2324 are not performed, the computer system can proceed from block 2314 to block 2326. In block 2326, the computer system can transmit, to the user device, the at least one insight for presentation in the GUI display of the user device, as described herein. The insight(s) can be specific to improve long-term metrics (e.g., sleep quality, sleep health, overall health) for the user and/or to help the user on the particular day (e.g., a day following a previous sleep session) or during a particular period of time (e.g., 1 day, 2 days, 3 days, a week, etc.). The insight(s), as described herein, can correspond to when to exercise, when the user is most alert, when the user should eat certain meals, and/or when the user should wind down before bed (e.g., personalized insights). The insight(s), as described herein, may also correspond to other more general tips and/or suggestions (e.g., generalized insights) that may educate or otherwise inform the user about their circadian chronotype, their circadian rhythm, and/or actions they can take that would maintain and/or improve their circadian rhythm.

As part of outputting the selected insight(s), the computer system can elevate at least one insight based on importance to the user (block 2328). Insights can be stackable, meaning that multiple insights can be presented to the user at once or over time. Each of the insights presented to the user can have varying degrees of importance to different aspects of the user's sleep health, sleep quality, and/or overall health. Sometimes, personalized insights, which can be specific ways in which the user can improve their sleep habits, can be stacked and presented above generalized insights, which can be more general educational tips about the user's circadian chronotype and/or circadian rhythm. Insights that may have a more direct impact and/or faster impact on maintaining the user's circadian rhythm and/or improving their sleep quality and/or health can be elevated over other insights. For example, insights to go to sleep earlier for a next sleep session can have a more direct impact on helping the user be more alert the day following that sleep session (this can be important to the user if they have a big meeting or big exam that day). Such insights can be elevated and presented to the user above insights that may have a more long-term impact on the user's sleep health (e.g., suggestions about when the user should exercise or eat dinner).

As part of outputting the selected insight(s), the computer system can generate and transmit a notification that prompts the user to perform activity that improves their overall health and/or sleep quality (block 2330). The notification can be presented at the user device, in the mobile application. The notification can also be presented at the user device as a push notification. Sometimes, the notification can be presented whenever the user opens the mobile application at the user device. The notification can be presented at predetermined times when the user opens the mobile application (e.g., when the user opens a particular screen in the mobile application, after a threshold amount of time passed since a last time the user opened the mobile application or a particular screen in the mobile application). The notification can be presented at predetermined times when the user does not open the mobile application (e.g., the user has not opened the mobile application between sleep sessions or for over a day, so the notification can remind the user to access the mobile application and/or perform one or more actions). The notification may also be presented at predetermined times based on what action is suggested/recommended by the selected insight(s). For example, if the insight recommends that the user exercises at 12:30 PM on a current day, the computer system can transmit a notification to the user device that alerts the user to exercise on the current day at or around 12:30 PM. The notification can be presented to the user a threshold amount of time before 12:30 PM (e.g., in the morning on the current day, 1 hour before 12:30 PM, 30 minutes before 12:30 PM, at 12:30 PM). Therefore, the notification can be used as a reminder that gently nudges the user to take action to change their habits, thereby maintaining their circadian rhythm and/or improving their overall sleep quality and/or health.

The computer system can also output the selected insight(s) in one or more other ways, as described throughout this disclosure.

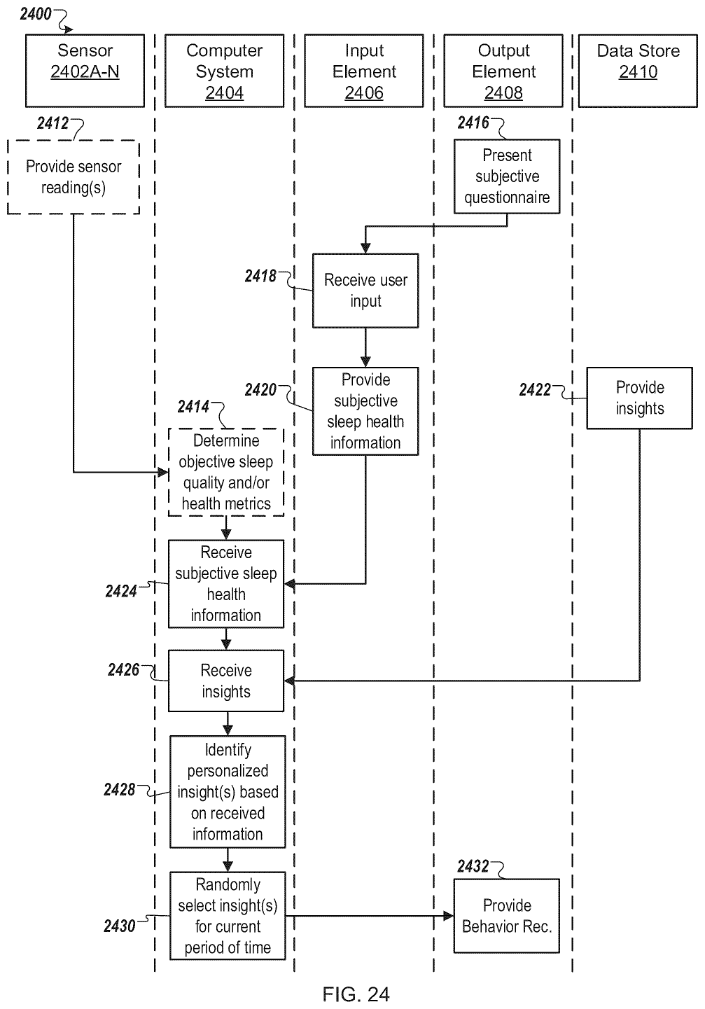

FIG. 24 is a swimlane diagram of a process 2400 for determining personalized insights for a user. The process 2400 can be performed to select one or more insights to present to the user, as described herein. Blocks in the process 2400 can be performed by one or more components described throughout this disclosure. For illustrative purposes, the process 2400 is described as being performed by components that include sensors 2402A-N (e.g., the sensors 1908A-N), a computer system 2404 (e.g., the computer system 1902), input element(s) 2406 (e.g., of the user device 1901), output element(s) 2408 (e.g., of the user device 1901), and a data store (2410). The blocks in the process 2400 may also be performed by one or more other components.

Referring to the process 2400 in FIG. 24, the sensors 2402A-N can optionally provide, to the computer system 2404, one or more sensor readings during and/or after a sleep session of the user (block 2412). Refer to block A-2 (1912) in FIG. 19 for further discussion.

The computer system 2404 can optionally determine one or more objective sleep quality and/or health metrics about the user based on the sensor reading(s) in block 2414. Refer to blocks 2320-2322 in the process 2300 of FIGS. 23A-B for further discussion.

Before, during, and/or after blocks 2412 and/or 2414 are performed, the output element 2408 can present a subjective questionnaire to the user (block 2416). The output element 2408 can be part of a computing device of the user, such as a mobile phone described herein. The output element 2408 can be a GUI display screen, such as a touchscreen. The questionnaire, as described throughout, can be presented in a mobile application, which the user can navigate to at their device. The questionnaire can be the health and wellness questionnaire described throughout this disclosure. Refer to FIGS. 21, 25, and 28 for further discussion.

The input element 2406 can receive user input in response to the questionnaire being presented to the user (block 2418). The input element 2406 can be part of the user's computing device. The input element 2406 can include, but is not limited to, a keyboard, mouse, microphone, and/or touchscreen display. As described herein, the user input can include responses to one or more questions presented in the questionnaire. Refer to block A-1 (1910) in FIG. 19 and FIGS. 23A-B, 25, and 28 for further discussion.

The input element 2406 can provide subjective sleep health information to the computer system 2402 (block 2420). The subjective sleep health information can include the user's answers to the questionnaire. The computer system 2404 can receive the subjective sleep health information in block 2424.

The data store 2410 can provide insights to the computer system 2404 in block 2422. The computer system 2404 can receive the insights (block 2426). Blocks 2422 and 2426 can be performed before, during, and/or after one or more of the blocks 2412, 2414, 2416, 2418, 2420, and 2424. The insights can include general and/or personalized insights described throughout this disclosure. The insights can be determined, by the computer system 2404 or another computing system, at another time and based on a variety of historic sleep and/or health data. The historic data can correspond to a general population of users (e.g., users who share one or more data/demographics/age/etc. with the user. The historic data may also correspond to the particular user.

As shown in FIG. 24, the computer system 2404 can receive the insights in block 2426, then select one or more of the received insights that correspond to the user's circadian chronotype (as determined based on the responses to the questionnaire and/or the sensor reading(s)). As another example, the computer system 2404 can determine the user's circadian chronotype based on the questionnaire answers and/or the sensor reading(s) and then retrieve only a set of the insights from the data store 2410 that correspond to the user's circadian chorotype.

Still referring to the process 2400 in FIG. 24, the computer system 2404 can identify one or more personalized insight(s) for the user based on the received information (block 2428). As part of identifying the insight(s), the computer system 2404 can determine the circadian chronotype of the user. As part of identifying the insight(s), the computer system 2404 can determine general insights for presentation to the user. The computer system 2404 may also determine or identify a combination of general and personalized insights for the user. Refer to FIGS. 19, 22, 23A-B, and 25-28 for further discussion.

The computer system 2404 can randomly select at least one insight for a current period of time (block 2430). The computer system 2404 can also select the insight(s) using one or more other logic and/or selection criteria, as described throughout this disclosure.

The computer system 2404 can provide the insight(s) to the output element 2408 for presentation as a behavior recommendation to the user (block 2432). The current period of time can be a day, a time between successive sleep sessions, an amount of time immediately following a previous sleep session, several days, a week, etc. The behavior recommendation can suggest one or more actions that the user can perform to maintain their circadian rhythm and/or improve their sleep quality and/or health. The behavior recommendation can suggest one or more times at which the user should perform one or more actions (e.g., exercise, eat a meal, wind down for bed, feeling or is most alert) to maintain their circadian rhythm and/or improve their sleep quality and/or health. The behavior recommendation may also include one or more general tips and/or suggestions about the user's circadian chronotype and actions that typically may affect their circadian rhythm and/or sleep quality and/or health. Refer to FIGS. 19, 20A-B, 22, 23A-B, and 25-28 for further discussion.

As described herein, one or more of the blocks 2412-2432 can be performed at different times. For example, blocks 2412 and 2414 can be performed during and/or after each sleep session of the user. Blocks 2416, 2418, 2420, and 2424 can be performed once, such as when the user creates an account with the service provided by the computer system 2404 and/or when the user sets up their bed system. Blocks 2422 and 2426 can be performed at a different time than blocks 2412 and 2414 and/or blocks 2416, 2418, 2420, and 2424. Sometimes, block 2428 can be performed once, such as whenever the computer system 2404 receives the subjective sleep health information in block 2424. Sometimes, block 2430 can be performed at predetermined time intervals, such as every day and/or after every sleep session (e.g., once the user wakes up for the day). Similarly, block 2432 can be performed at predetermined time intervals, including but not limited to, whenever the user opens the mobile application at the user's device, whenever the user wakes up from a sleep session, after a threshold amount of time passed between sleep sessions, after a threshold amount of time passed since the user last opened the mobile application, etc. One or more of the blocks 2412-2432 can also be performed at one or more other times as described herein.

FIG. 25 is a flowchart of a process 2500 for providing personalized insights to a user in a GUI. The process 2500 can be used to present a questionnaire to the user at one time. In other words, a subjective sleep health questionnaire described throughout this disclosure (e.g., refer to FIG. 21) can be presented in the GUI display of a user device in a single instance. The questionnaire can be completed once by the user. The user can, however, access the questionnaire in a mobile application at the user device at one or more later/other times to complete questions they did not previously answer and/or update/modify their previously-provided responses. For illustrative purposes, the process 2500 is described from the perspective of a computer system.

Referring to the process 2500 in FIG. 25, the computer system can present a questionnaire to a user at a user device in block 2502. As described herein, the questionnaire can be accessible from a screen in the mobile application presented at the user device. The user can access the questionnaire whenever they desire. In some implementations, the computer system can transmit a notification to the user device that prompts the user to access and respond to the questionnaire. Similarly, the computer system can simply transmit the questionnaire for presentation in the notification at the user device. Refer to FIGS. 19, 21, 23A-B, 24, and 28 for further discussion.

In block 2504, the computer system can receive user input in response to the questionnaire. As described herein, the user can respond to questions in the questionnaire once the user accesses the questionnaire. The user can respond to the questions when the questionnaire is automatically presented at the user device. The user can also respond to the questionnaire some period of time after the questionnaire is initially presented in block 2502. For example block 2502 can be performed at a first time, when the user sets up their bed. Block 2504 can then be performed several days after the user sets up their bed, once the user reviews the questionnaire and decides they have answers or would like to answer. As described herein, the user can also provide updated user input or new user input to the questionnaire at one or more other times. For example, the user can decide to update any of their previously provided responses to the questionnaire over time. The user can also decide to provide responses for questions they had not previously responded to. The user may also decide to remove/delete one or more of their previously provided responses. Refer to FIGS. 19, 23A-B, 24, and 28 for further discussion.

The computer system can determine a user circadian chronotype based on the user input in block 2506. The computer system can perform block 2506 whenever the computer system receives user input or updated user input in block 2504. Refer to FIGS. 19, 23A-B, 24, and 28 for further discussion.

The computer system can identify one or more behavior recommendations based on the circadian chronotype (block 2508). The computer system can perform block 2508 whenever the computer system receives user input in block 2504 and/or determines the circadian chronotype of the user in block 2506. Refer to FIGS. 19, 22, 23A-B, 24, and 28 for further discussion.

The computer system can present the behavior recommendation(s) at the user device in block 2510. The computer system can present the recommendation(s) at predetermined time intervals, as described herein. Sometimes, the computer system can present the recommendation(s) whenever the computer system performs blocks 2506 and/or 2508. Refer to FIGS. 19, 20A-B, 23A-B, 24, and 28 for further discussion.

The computer system can wait to receive any additional user input from the user in block 2504. In other words, the computer system may not present the questionnaire another time or prompt the user to complete the questionnaire. Rather, the computer system can wait for the user to access the questionnaire at another time and provide new responses, update previous responses, and/or complete questions they had not previously completed in the questionnaire. If and when the user provides updated user input at the user device, the computer system can perform the blocks 2504-2510 or any other combination or subset of those blocks.

FIG. 26 is a flowchart of a process 2600 for automatically determining a circadian chronotype of a user. The process 2600 can be performed to determine the user's circadian chronotype without receiving and using user input, such as responses to a questionnaire as described herein. Accordingly, the circadian chronotype can be determined based on sleep patterns of the user over time and/or other historic sleep and/or health data for the user. The historic data of the user can be mapped to one or more different types of chronotypes described herein. For illustrative purposes, the process 2600 is described from the perspective of a computer system.

Referring to the process 2600 in FIG. 26, the computer system can retrieve sleep patterns and behavior data for a user in block 2602. In other words, the computer system can retrieve, from a data store, historic sleep data about the user. The historic sleep data can include sleep patterns and/or behaviors of the user over a threshold period of time. The data can be collected by sensors or other components of the user's bed system, as described herein. The data can also be generated by the computer system or other computing devices based on processing one or more different sensor data. The data can correspond to multiple sleep sessions, one sleep session, a prior or last sleep session, one week, a couple days, a last day, etc. The data may include, but is not limited to, sleep scores, breathing patterns, heartrate, respiration rate, snore, other sleep and/or health information sensed and/or determined by components described throughout this disclosure.

The computer system can apply a ruleset to the retrieved data to determine a circadian chronotype (e.g., a proposed circadian chronotype) of the user (block 2604). In some implementations, the computer system may apply one or more machine learning trained models and/or algorithms to the retrieved data. Determining the circadian chronotype of the user can include mapping the sleep patterns and behavior data (or other historic data) for the user to at least one circadian chronotype that is defined by the ruleset. The ruleset can define circadian chronotypes that include night owl, early bird, and neutral. One or more other circadian chronotypes can be defined in the ruleset that can be mapped to the user's data.

The computer system can determine insights, tips, and/or reminders for the user that correspond to the circadian chronotype of the user in block 2606. For example, as described herein, the computer system can generate (e.g., using templates) and/or identify (e.g., from a set of existing insights that correspond to the circadian chronotype and were previously determined by the computer system) alerts that can be provided to the user (at a user device of the user) to optimize their sleep schedule and/or circadian rhythm. The computer system can also generate notifications, tips, or insights that provide recommendations for creating and/or updating bedtime routines and/or reminders based on the user's circadian chronotype. The computer system may generate, based on the user's circadian chronotype, one or more reminders that gently nudge the user of a time at which the user should wind down for bed and/or go to bed (and/or a predetermined period of time in which the user should wind down and/or go to bed each night). Accordingly, the computer system can leverage the historic data about the user (e.g., prior sleep patterns) to generate recommendations that can help the user maintain and/or establish a consistent sleep schedule.

The computer system can then return the circadian chronotype and/or the insights, tips, and/or reminders in block 2608. As described herein, such information can be presented in a mobile application and/or the GUIs described herein at the user's device. For example, the computer system can output this information at the user device of the user (block 2610).

As another example, the computer system can generate instructions to control components of the bed of the user and/or peripheral devices according to the insights, tips, and/or reminders (block 2612). For example, the computer system can generate a recommendation to change a temperature of the user's bed to help the user fall asleep faster. Accordingly, the computer system can generate instructions that cause a heating/cooling unit of the bed to automatically adjust the temperature of the bed by a predetermined threshold amount before and/or while the user is sleeping. As another example, the computer system can generate a recommendation to adjust a foundation of the bed to help the user fall asleep faster. The computer system can then generate instructions that cause a controller of the bed to adjust the foundation to a predetermined position before the user gets into bed and/or when the user is ready to go to sleep. As another example, the computer system can generate instructions that cause a home automation device to dim lights in a bedroom or other rooms of the user's house at predetermined times (e.g., earlier in the evening than typical) to help the user wind down before bed to then wake up fresher and more alert. One or more other instructions can be generated and/or executed based on the circadian chronotype, to then be performed by components in the user's sleep environment, as described above.

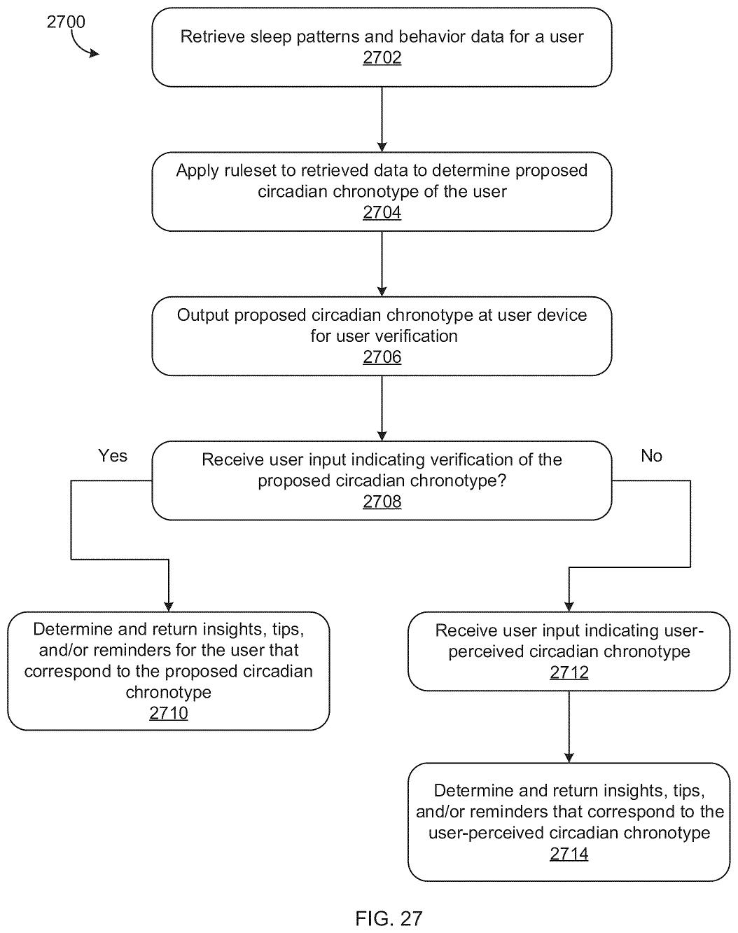

FIG. 27 is a flowchart of another process 2700 for automatically determining a circadian chronotype of a user. The process 2700 can be performed to verify a system-determined circadian chronotype with user input, where the user input indicates a user-perceived circadian rhythm or chronotype. The process 2700 can leverage historic sleep data about the user (e.g., prior sleep patterns, bed presence, when the user falls asleep, how often they wake up during a sleep session) to propose a circadian chronotype for the user, then present the proposed circadian chronotype to the user to be confirmed/updated. The user can, for example, provide input indicating that they agree with the proposed circadian chronotype. The user can also provide input indicating that they believe they have a different circadian chronotype (e.g., the proposed circadian chronotype can be neutral but the user can provide responses to a questionnaire that indicate the user is actually a night owl). For illustrative purposes, the process 2700 is described from the perspective of a computer system.

Referring to the process 2700, the computer system can retrieve sleep patterns and behavior data for a user in block 2702. Refer to block 2602 in FIG. 26 for further discussion.

The computer system can apply a ruleset to the retrieved data to determine a proposed circadian chronotype of the user in block 2704. Refer to block 2604 in FIG. 26 for further discussion.

The computer system can output the proposed circadian chronotype at a user device for user verification (block 2706). The proposed circadian chronotype can be outputted in a GUI display of the user device. For example, a notification can be presented at the user device that indicates the circadian chronotype and asks the user whether this is a correct chronotype or not for the user. Sometimes, instead of outputting the proposed circadian chronotype, the computer system can output a sleep and health questionnaire as described herein. The computer system can then receive user input to the questionnaire, which can be processed by the computer system to determine whether the proposed circadian chronotype is actually a correct circadian chronotype of the user.

The computer system may receive user input and determine whether the received input indicates verification of the proposed circadian chronotype (block 2708). The user input can include selection of an option (e.g., button) that verifies the proposed circadian chronotype. The user input can include selection of an option that disagrees with the proposed circadian chronotype. The user input can also include responses to one or more questions in the questionnaire. The computer system can process the user input to determine a circadian chronotype of the user based on the user input. Then, the computer system can determine whether that circadian chronotype matches with or corresponds to the proposed circadian chronotype.

If the user input indicates verification of the proposed circadian chronotype, then the computer system can determine and return insights, tips, and/or reminders for the user that correspond to the proposed circadian chronotype in block 2710. Refer to blocks 2608-2612 in FIG. 26 for further discussion.

If the user input does not indicate verification of the proposed circadian chronotype, then the computer system may receive user input indicating a user-perceived circadian chronotype in block 2712. The user-perceived circadian chronotype can be different than the outputted circadian chronotype. For example, if the proposed circadian chronotype is night owl, the user can provide input indicating that they perceive their circadian chronotype to be that of an early bird or neutral. In some implementations, the computer system may not receive the user-perceived circadian chronotype in block 2712. Instead, the computer system can determine the circadian chronotype of the user based on the user responses to the questionnaire, using the techniques described above.

The computer system can then determine and return insights, tips, and/or reminders that correspond to the user-perceived circadian chronotype in block 2714. Refer to discussion above for additional information.

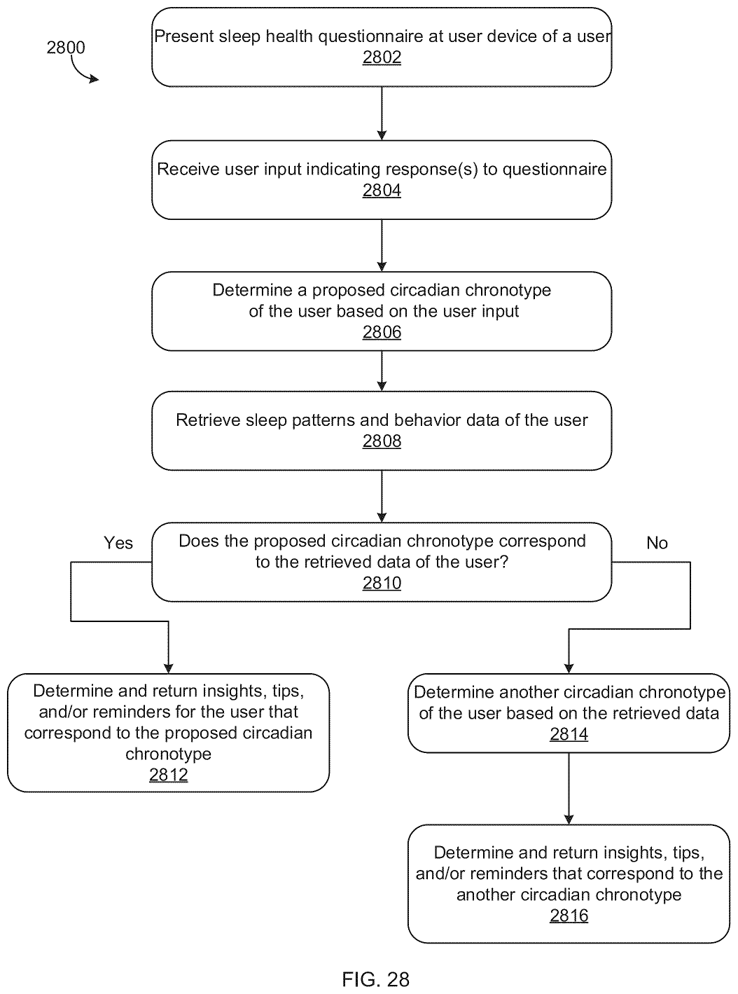

FIG. 28 is a flowchart of a process 2800 for verifying a user-perceived circadian chronotype with automated analysis of historic sleep data of a user. In the process 2800, the user can answer questions, such as the questionnaire described herein (e.g., refer to FIG. 21). The questions may ask the user what they believe their circadian chronotype is. A computer system can process the user's answers to propose what the user's circadian chronotype may be (e.g., the computer system can identify the proposed circadian chronotype as the user-perceived circadian chronotype, the computer system can determine the proposed circadian chronotype based on mapping the user's answers to the questions to a defined circadian chronotype). The computer system can check the proposed circadian chronotype against historic sleep data and/or health data about the user (and/or a general population of users that share same/similar characteristics with the user) to verify or validate the user-perceived circadian chronotype and/or the proposed circadian chronotype for the user. For illustrative purposes, the process 2500 is described from the perspective of a computer system.

Referring to the process 2800, the computer system can present a sleep health questionnaire at a user device of a user, as described throughout this disclosure (block 2802). The computer system can receive user input indicating one or more responses to the questionnaire (block 2804). The computer system can determine a proposed circadian chronotype of the user based on the user input in block 2806. Refer to FIGS. 19 and 23-25 for further discussion. The computer system can also retrieve sleep patterns and behavior data of the user in block 2808. Refer to FIGS. 26-27 for further discussion. The computer system can determine whether the proposed circadian chronotype corresponds to the retrieved data of the user (block 2810), as described further in FIG. 27. If the proposed circadian chronotype corresponds to the data about the user (e.g., the chronotype is verified), then the computer system can determine and return insights, tips, and/or reminders for the user that correspond to the proposed circadian chronotype (block 2812). If the proposed circadian chronotype does not correspond to the data about the user, then the computer system can determine another circadian chronotype of the user based on the retrieved data in block 2814. The computer system can then determine and return insights, tips, and/or reminders that correspond to the another circadian chronotype in block 2816. Refer to FIG. 27 for further discussion. In some implementations, the computer system can instead determine insights, tips, and/or reminders for the user that correspond to the user-perceived circadian chronotype provided as input to the questionnaire.

What is claimed is:

1. A system for improving at least one of a sleep quality and a health metric of a user, the system comprising:
a computer system in communication with a user device of a user, the computer system configured to:
transmit, to the user device, a subjective sleep health questionnaire for presentation in a graphical user interface (GUI) display;
receive, from the user device, user input indicating at least one response to the subjective sleep health questionnaire;
process the user input to determine a circadian chronotype of the user;
identify, based on the circadian chronotype of the user, at least one insight for presentation in the GUI display at the user device; and
transmit, to the user device, the at least one insight for presentation in the GUI display.

2. The system of claim 1, wherein the at least one insight is a behavior recommendation to improve a current circadian rhythm of the user over a predetermined period of time.

3. The system of claim 1, wherein identifying the at least one insight comprises:
retrieving, from a data store, sleep and health insights associated with the circadian chronotype of the user;
identifying, based on the processed user input, a subset of insights from the retrieved sleep and health insights; and
selecting, using a randomization technique, the at least one insight from the subset of insights for presentation to the user.

4. The system of claim 1, wherein the questionnaire prompts the user for user input about at least one of user-perceived circadian rhythm, user-perceived stress, user-perceived trouble falling asleep, and user-perceived trouble staying asleep.

5. The system of claim 1, wherein the at least one insight includes a recommendation of when the user should exercise.

6. The system of claim 1, wherein the at least one insight includes a recommendation of when the user is most alert during a predetermined period of time.

7. The system of claim 1, wherein the at least one insight includes a recommendation of when the user should eat a meal.

8. The system of claim 1, wherein the at least one insight includes a recommendation of when the user should begin a bedtime routine.

9. The system of claim 1, wherein the at least one insight includes a notification prompting the user to perform an activity that improves at least one of a current health metric and a current sleep quality of the user.

10. The system of claim 1, wherein the subjective sleep health questionnaire prompts the user for an indication of whether the user is an early bird, a night owl, or neutral.

11. The system of claim 1, wherein the subjective sleep health questionnaire prompts the user for an indication of at least one factor that disrupts sleep patterns of the user.

12. The system claim 1, wherein to select at least one insight from the subset of insights, the computer system is configured to assemble the subset of insights from a template of general insights that have been completed with information generic to a population of users that include the user.

13. A system for improving at least one of a sleep quality and a health metric of a user, the system comprising:
a computer system in communication with a user device of a user, the computer system configured to:
retrieve, from a data store, sleep patterns and behavior data for the user;
apply a ruleset to the retrieved data to determine a circadian chronotype of the user;
determine at least one insight, tip, or reminder for the user, wherein the at least one insight, tip, or reminder corresponds to the circadian chronotype of the user; and
return at least one of (i) the circadian chronotype and (ii) the determined insight, tip, or reminder.

14. The system of claim 13, wherein the returning comprises transmitting, to the user device, at least one of (i) and (ii) for presentation in a GUI display of the user device.

15. The system of claim 13, wherein the returning comprises generating instructions to control a component of a bed system or a peripheral device according to the at least one insight, tip, and reminder.

16. The system of claim 13, wherein determining the circadian chronotype of the user comprises mapping the sleep patterns and behavior data for the user to at least one circadian chronotype defined by the ruleset.

17. The system of any of the claim 13, wherein the computer system is further configured to:
output, in a GUI display of the user device, the circadian chronotype of the user;
receive user input, from the user device, indicating verification of the circadian chronotype; and
in response to receiving the user input, perform the determining step.

18. The system of claim 13, wherein the computer system is further configured to:
output, in a GUI display of the user device, the circadian chronotype of the user;
receive user input, from the user device, indicating a user-perceived circadian chronotype that is different than the outputted circadian chronotype; and
in response to receiving the user input, determine the at least one insight, tip, and reminder for the user that corresponds to the user-perceived circadian chronotype.

19. A system for improving at least one of a sleep quality and a health metric of a user, the system comprising:
a computer system in communication with a user device of a user, the computer system configured to:
present a sleep health questionnaire at the user device;
receive user input, from the user device, indicating at least one response to the questionnaire;
determine, based on the user input, a proposed circadian chronotype of the user;
retrieve, from a data store, sleep patterns and behavior data for the user;
determine whether the retrieved sleep patterns and behavior data corresponds to the proposed circadian chronotype of the user;
determine, based on the retrieved sleep patterns and behavior data corresponding to the proposed circadian chronotype, at least one insight, tip, or reminder for the user, wherein the determined insight, tip, or reminder corresponds to the proposed circadian chronotype; and
return at least one of (i) the proposed circadian chronotype and (ii) the insight, tip, or reminder.

20. The system of claim 19, wherein the computer system is configured to:
determine, based on the retrieved data not corresponding to the proposed circadian chronotype, another circadian chronotype of the user based on the retrieved data;
determine at least one insight, tip, and reminder for the user that corresponds to the another circadian chronotype; and
return at least one of (i) the another circadian chronotype and (ii) the insight, tip, or reminder that corresponds to the another circadian chronotype.
